(19) United States
(12) Patent Application Publication
(10) Pub. No.: US 2023/0358770 A1
(43) Pub. Date: Nov. 9, 2023

(54) BIOSENSORS FOR DETECTING CHANGES IN THE LEVEL OF A NEUROTRANSMITTER IN THE CENTRAL NERVOUS SYSTEM

(71) Applicants: Paul Feinstein, New York, NY (US); Charlotte D'Hulst, Borgerhout (BE)

(72) Inventors: Paul Feinstein, New York, NY (US); Charlotte D'Hulst, Borgerhout (BE); Elke Giets, Nossegem (BE); Masayo Omura, Brooklyn, NY (US); Christopher Lemetre, Sloatsburg, NY (US)

(21) Appl. No.: 18/245,992

(22) PCT Filed: Sep. 22, 2021
(86) PCT No.: PCT/US2021/051586

§ 371 Date: Mar. 20, 2023

Related U.S. Application Data
(60) Provisional application No. 63/081,871, filed on Sep. 22, 2020.

(51) Int. Cl. G01N 33/94; C12N 5/0793; G01N 33/50; A01K 67/027; C07K 14/705; C12N 15/86

(52) U.S. Cl. CPC G01N 33/9406; C12N 5/062; G01N 33/5091; A01K 67/0275; C07K 14/705; C12N 15/86; G01N 33/9413; G01N 2800/28; G01N 2333/70571; C12N 2740/15043; A01K 2217/052; A01K 2227/105; A01K 2267/0393; G01N 2800/14

(57) ABSTRACT

Provided herein are biosensors and methods for detecting one or more odorants associated with the levels, or a change in the levels, of one or more neurotransmitters in the central nervous system of a subject. In embodiments, provided are biosensors that comprise one or more populations of olfactory neurons, or cilia derived therefrom, wherein each population preferentially expresses a specific odorant receptor (OR). Also provided are biosensors comprising a cell or a population of cells engineered to express certain ORs; biosensors comprising certain isolated ORs; transgenic animals and tissues derived therefrom that preferentially express certain ORs; isolated cells or populations of cells engineered to express certain ORs; expression constructs for the preferential expression of certain ORs; and methods of using the biosensors, transgenic animals, tissues, cells, population of cells, and expression constructs disclosed herein.

### CROSS-REFERENCE TO RELATED APPLICATION

This application claims priority to U.S. Provisional Application No. 63/081,871, filed Sep. 22, 2020, which is incorporated herein by reference in its entirety.

### SEQUENCE LISTING

A Sequence Listing is provided herewith as a text file, “Sequence listing_ST25” created on Sep. 22, 201, and having a size of 160 kb. The contents of the text file are incorporated by reference herein in their entirety.

### FIELD

The present disclosure provides biosensors and methods for detecting one or more odorants associated with a level or a change in the levels of one or more neurotransmitters, in particular dopamine deficiency, in the central nervous system of a subject. More particularly, the disclosure relates to biosensors comprising one or more populations of olfactory sensory neurons, or cilia derived therefrom, where each population preferentially expresses an odorant receptor.

### BACKGROUND

Neurotransmitters are chemical agents released by neurons to stimulate neighboring neurons or muscle or gland cells, thus allowing electrical impulses to be passed from one cell to the next throughout the nervous system. For example, the neurotransmitter dopamine is responsible for transmitting signals within the brain that allow for coordination of movement. Accordingly, a dopamine imbalance in the brain can negatively affect mood, sleep, memory, learning, concentration, and motor control. Dopamine deficiency is related to a number of diseases and conditions, including, but not limited to, Parkinson's disease (PD), depression, schizophrenia, dystonia, and restless leg syndrome.

PD is a neurodegenerative disorder affecting at least one million people in the United States, and more than five million worldwide. PD is associated with disruption of dopamine-producing (“dopaminergic”) neurons in the brain, in particular in an area called the substantia nigra. Alteration of dopamine production causes neurons to fire without normal control, leaving patients less able to direct or control their movement. Symptoms of dopamine alteration in PD generally develop slowly over years and include movement-related (“motor”) symptoms such as tremor; slowness of movements (bradykinesia); stiffness or rigidity of the arms, legs or trunk; and gait and balance problems. In addition, some PD symptoms may be unrelated to movement (“non-motor” symptoms), and can include apathy, depression, constipation, sleep behavior disorders, anosmia (loss of sense of smell), and cognitive impairment.

There is no objective test or biomarker (such as a blood test, brain scan, or electroencephalogram) to make a definitive diagnosis of PD or the associated dopamine alteration. Rather, a diagnosis of PD is made following ascertainment of the patient's medical history and a thorough neurological examination, looking in particular for two or more of the cardinal late onset symptoms—tremor, bradykinesia, rigidity of the limbs or trunk, and balance trouble. Additionally, a doctor may also look for responsiveness to PD medications as further evidence that PD is the correct diagnosis. Unfortunately, the rates for misdiagnosing PD or failing to diagnose PD are high, because there is no definitive test for PD, and because PD symptoms are similar to those of other neurological conditions. Importantly, current standard methods for the diagnosis of PD are limited to the identification of PD after symptoms of PD have already manifested in the patient. Conversely, these PD symptoms occur only after a significant amount of the substantia nigra neurons have already been lost or impaired.

Accordingly, new devices and methods that facilitate a determination of whether or not a patient exhibits a change in the levels of one or more neurotransmitters (including, but not limited to dopamine) in the central nervous system (CNS) are urgently needed.

### SUMMARY

In one aspect, provided is a biosensor comprising one or more populations of olfactory sensory neurons (OSNs), or cilia derived therefrom; wherein each population of OSNs preferentially expresses an OR comprising an amino acid sequence selected from the group consisting of SEQ ID NOs: 1-40, or an amino acid sequence with greater than 85% identity (e.g., greater than 90%, greater than 95%, greater than 96%, greater than 97%, greater than 98%, or greater than 99% identity) to any one of SEQ ID NOs: 1-40. In embodiments, provided is a biosensor comprising one or more populations of OSNs, or cilia derived therefrom; wherein each population of OSNs preferentially expresses an OR comprising an amino acid sequence selected from the group consisting of SEQ ID NOs: 1 and 6-18, or an amino acid sequence with greater than 85% identity (e.g., greater than 90%, greater than 95%, greater than 96%, greater than 97%, greater than 98%, or greater than 99% identity) to any one of SEQ ID NOs: 1 and 6-18. In embodiments, provided is a biosensor comprising one or more populations of OSNs, or cilia derived therefrom; wherein each population of OSNs preferentially expresses an amino acid sequence comprising an OR described in any of Tables 1-5. In one aspect, provided is a biosensor comprising one or more populations of OSNs, or cilia derived therefrom; wherein each population of OSNs preferentially expresses an OR comprising an amino acid sequence with greater than 85% identity (e.g., greater than 90%, greater than 95%, greater than 96%, greater than 97%, greater than 98%, or greater than 99% identity) to any of the amino acid sequences of an OR described in any of Tables 1-5. In embodiments, the one or more populations of OSNs, or cilia derived therefrom, are attached to a solid support. In some embodiments, the solid support is selected from the group consisting of silicon, glass, polystyrene, and polymers.

In embodiments, the one or more populations of OSNs further express a marker for detecting activation or lack of activation of the OR. In embodiments, the one or more populations of OSNs further express one or more markers for detecting activation or lack of activation of the OR, wherein the activation, if any, occurs upon exposure of the one or more populations of OSNs, or cilia derived therefrom, to a sample, or and extract of the sample, from a subject. In some embodiments, the markers is a calcium-sensitive fluorescent dye selected from the group consisting of fura-2, fluo-3, fluo-4, fluo-5F, indo-1, and Oregon Green BAPTA. In some embodiments, the marker is selected from the group consisting of GECO2.1, GCaMP6, Flamindo, Flamindo2, and Pink Flamindo. In some embodiments, the marker for detecting activation or lack of activation of the OR is co-expressed with the preferentially expressed odorant receptor (OR).

In embodiments, the OSNs comprise an enhancer operably linked to the sequence encoding the preferentially expressed OR. In embodiments, the enhancer is a singular gene choice enhancer. In some embodiments, the enhancer comprises at least four sequential repeats of a 21 base pair (bp) sequence wherein each 21 bp sequential repeat comprises the sequence AACTTTTTAATGA (SEQ ID NO: 81). In some embodiments, the singular gene choice enhancer sequence comprises at least four sequential repeats of a 21 bp sequence wherein each 21 bp sequential repeat comprises the sequence AACTTTTTAATGA (SEQ ID NO: 81). In some embodiments, the enhancer comprises at least four sequential repeats of ACATAACTTTTTAATGAGTCT (SEQ ID NO: 82). In some embodiments, the singular gene choice enhancer sequence comprises at least four sequential repeats of ACATAACTTTTTAATGAGTCT (SEQ ID NO: 82). In some embodiments, the enhancer comprises ten or fewer of the 21 bp sequential repeats. In some embodiments, the singular gene choice enhancer comprises ten or fewer of the 21 bp sequential repeats. In some embodiments, the enhancer comprises one or more TetO sequences. In some embodiments, the singular gene choice enhancer comprises one or more TetO sequences.

In aspects, provided is a biosensor comprising a cell or population of cells engineered to express an OR comprising an amino acid sequence selected from the group consisting of SEQ ID NOs: 1-40, or an amino acid sequence with greater than 85% identity (e.g., greater than 90%, greater than 95%, greater than 96%, greater than 97%, greater than 98%, or greater than 99% identity) to any one of SEQ ID NOs: 1-40. In aspects, provided is a biosensor comprising a cell or population of cells engineered to express an OR comprising (i) an amino acid sequence selected from the group consisting of SEQ ID NOs: 1 and 6-18, (ii) an amino acid sequence with greater than 85% identity (e.g., greater than 90%, greater than 95%, greater than 96%, greater than 97%, greater than 98%, or greater than 99% identity) to any one of SEQ ID NOs: 1 and 6-18, (iii) an amino acid sequence of an OR described in any of Tables 1-5, and/or (iv) an amino acid sequence with greater than 85% identity (e.g., greater than 90%, greater than 95%, greater than 96%, greater than 97%, greater than 98%, or greater than 99% identity) to an amino acid sequence of an OR described in any of Tables 1-5. In some embodiments, the cell is a eukaryotic cell or the population of cells is a population of eukaryotic cells. In some embodiments, the cell or the cells in the population of eukaryotic cells are yeast cells or OSNs. In some embodiments, the cell or the population of cells further expresses one or more markers for detecting activation or lack of activation of the OR. In some embodiments, the cell or population of cells further expresses one or more markers for detecting activation or lack of activation of the OR, wherein the activation occurs upon exposure of the cell or population of cells to a sample from a subject with a change in the levels of one or more neurotransmitters in the CNS (e.g., as compared to the control levels for the one or more neurotransmitters). In some embodiments, the marker is a-sensitive fluorescent dye selected from the group consisting of fura-2, fluo-3, fluo-4, fluo-5F, indo-1, and Oregon Green BAPTA. In some embodiments, the marker is selected from the group consisting of GECO2.1, GCaMP6, Flamindo, Flamindo2, and Pink Flamindo. In some embodiments, the marker for detecting activation or lack of activation of the OR is co-expressed with the expressed OR.

In aspects, the biosensors described herein are for use in detecting one or more odorants, or a change in the levels of one or more odorants (e.g., as compared to control levels of the one or more odorants), in a sample from a subject. In embodiments, the one or more odorants, or the change in the levels of the one or more odorants, is associated with the levels of one or more neurotransmitters, or a change in the levels of one or more neurotransmitters (e.g., as compared to control levels of the one or more neurotransmitters), in the CNS of a subject. Thus, in embodiments, the biosensors described herein are for use in detecting the levels of one or more neurotransmitters, or a change in the levels of one or more neurotransmitters (e.g., as compared to control levels of the one or more neurotransmitters), in the CNS of a subject. The one or more neurotransmitters can include, for example, dopamine, norepinephrine (noradrenaline), epinephrine (adrenaline), histamine, and/or serotonin. In embodiments, the one or more odorants, or the change in the levels of the one or more odorants, is associated with a disease characterized by a change in the levels of one or more neurotransmitters in the CNS (e.g., as compared to control levels of the one or more neurotransmitters), including, e.g., a disease associated with a dopamine deficiency in the CNS such as PD, depression, schizophrenia, dystonia, or restless leg syndrome. Thus, in embodiments, the biosensors described herein are for use in detecting a disease characterized by a change in the levels of one or more neurotransmitters in the CNS including, e.g., a disease associated with a dopamine deficiency in the CNS such as PD, depression, schizophrenia, dystonia, or restless leg syndrome. In embodiments, the one or more odorants, or the change in the levels of the one or more odorants, detected by the biosensors disclosed herein are associated with a neurodegenerative disease in the subject. Thus, in embodiments, the biosensors described herein are for use in detecting neurodegenerative disease in the subject. In embodiments, the neurodegenerative disease is PD.

In one aspect, provided is a transgenic animal, or a tissue, cell, or population of cells isolated from the transgenic animal, wherein the transgenic animal comprises an olfactory epithelium in which the OSNs preferentially express an OR comprising an amino acid sequence selected from the group consisting of SEQ ID NOs: 1-40, or an amino acid sequence with greater than 85% identity (e.g., greater than 90%, greater than 95%, greater than 96%, greater than 97%, greater than 98%, or greater than 99% identity) to any one of SEQ ID NOs: 1-40. In one aspect, provided is a transgenic animal, or a tissue, cell, or population of cells isolated from the transgenic animal, wherein the transgenic animal comprises an olfactory epithelium in which the OSNs preferentially express an OR comprising an amino acid sequence selected from the group consisting of (i) SEQ ID NOs: 1 and 6-18, (ii) an amino acid sequence with greater than 85% identity (e.g., greater than 90%, greater than 95%, greater than 96%, greater than 97%, greater than 98%, or greater than 99% identity) to any one of SEQ ID NOs: 1 and 6-18, (iii) an amino acid sequence of an OR described in any of Tables 1-5, and/or (iv) an amino acid sequence with greater than 85% identity (e.g., greater than 90%, greater than 95%, greater than 96%, greater than 97%, greater than 98%, or greater than 99% identity) to an amino acid sequence of an OR described in any of Tables 1-5. In embodiments, the isolated tissue is an olfactory epithelium. In embodiments, the isolated cell or population of cells is an olfactory epithelium cell or population of olfactory epithelium cells (e.g., an OSN or a population of OSNs that preferentially express an OR described herein).

In one aspect, provided is a transgenic animal, or a tissue, cell, or population of cells isolated from the transgenic animal, wherein the transgenic animal, or the tissue, cell, or population of cells isolated from the transgenic animal, comprises: (a) a transgene sequence encoding an OR comprising an amino acid sequence selected from the group consisting of SEQ ID NOs: 1-40, or an amino acid sequence with greater than 85% identity (e.g., greater than 90%, greater than 95%, greater than 96%, greater than 97%, greater than 98%, or greater than 99% identity) to any one of SEQ ID NOs: 1-40; and (b) an enhancer operably linked to the transgene sequence. In one aspect, provided is a transgenic animal, or a tissue, cell, or population of cells isolated from the transgenic animal, wherein the transgenic animal, or the tissue, cell, or population of cells isolated from the transgenic animal, comprises: (a) a transgene sequence encoding an OR comprising an amino acid sequence selected from the group consisting of (i) SEQ ID NOs: 1 and 6-18, (ii) an amino acid sequence with greater than 85% identity (e.g., greater than 90%, greater than 95%, greater than 96%, greater than 97%, greater than 98%, or greater than 99% identity) to any one of SEQ ID NOs: 1 and 6-18, (iii) an amino acid sequence of an OR described in any of Tables 1-5, and/or (iv) an amino acid sequence with greater than 85% identity (e.g., greater than 90%, greater than 95%, greater than 96%, greater than 97%, greater than 98%, or greater than 99% identity) to an amino acid sequence of an OR described in any of Tables 1-5; and (b) an enhancer operably linked to the transgene sequence. In embodiments, the enhancer is a singular gene choice enhancer. In embodiments, the enhancer comprises at least four sequential repeats of a 21 base pair (bp) sequence wherein each 21 bp sequential repeat comprises the sequence AACTTTTTAATGA (SEQ ID NO: 81). In some embodiments, the enhancer comprises at least four sequential repeats of ACATAACTTTTTAATGAGTCT (SEQ ID NO: 82). In embodiments, the enhancer comprises ten or fewer of the 21 bp sequential repeats. In embodiments, the singular gene choice enhancer comprises ten or fewer of the 21 bp sequential repeats. In some embodiments, the enhancer comprises one or more TetO sequences. In some embodiments, the singular gene choice enhancer comprises one or more TetO sequences.

In some embodiments, the transgenic animal is a non-human mammal. In some embodiments, the non-human mammal belongs to the family of Bovidae, Canidae, or Muridae. In some embodiments, the non-human mammal is a rat, a mouse, a dog, cat, goat, chicken, sheep, pig, or primate.

In aspects, the transgenic animal, or a tissue, cell, or population of cells isolated from the transgenic animal, described herein are for use in detecting one or more odorants, or a change in the levels of one or more odorants (e.g., as compared to control levels of the one or more odorants), in a sample from the subject. In embodiments, the one or more odorants, or the change in the levels of the one or more odorants, is associated with the levels of one or more neurotransmitters, or a change in the levels of one or more neurotransmitters (e.g., as compared to control levels of the one or more neurotransmitters), in the CNS of a subject. Thus, in embodiments, the transgenic animals, or a tissue, cell, or population of cells isolated from the transgenic animal described herein are for use in detecting the levels of one or more neurotransmitters, or a change in the levels of one or more neurotransmitters (e.g., as compared to control levels of the one or more neurotransmitters), in the CNS of a subject. The one or more neurotransmitters can include, for example, dopamine, norepinephrine (noradrenaline), epinephrine (adrenaline), histamine, and/or serotonin.

In embodiments, the one or more odorants, or the change in the levels of the one or more odorants, detected using a transgenic animal, or a tissue, cell, or population of cells isolated from the transgenic animal disclosed herein are associated with a neurodegenerative disease in the subject. Thus, in embodiments, the transgenic animal, or a tissue, cell, or population of cells isolated from the transgenic animal described herein are for use in detecting a neurodegenerative disease in the subject. In embodiments, the neurodegenerative disease is PD.

In one aspect, provided is an expression construct comprising: an OR coding sequence, wherein the OR coding sequence encodes an amino acid sequence selected from the group consisting of SEQ ID NOs: 1-40, or an amino acid sequence with greater than 85% identity (e.g., greater than 90%, greater than 95%, greater than 96%, greater than 97%, greater than 98%, or greater than 99% identity) to any one of SEQ ID NOs: 1-40. In embodiments, provided is an expression construct comprising an OR coding sequence, wherein the OR coding sequence encodes an amino acid sequence selected from the group consisting of SEQ ID NOs: 1 and 6-18, or an amino acid sequence with greater than 85% identity (e.g., greater than 90%, greater than 95%, greater than 96%, greater than 97%, greater than 98%, or greater than 99% identity) to any one of SEQ ID NOs: 1 and 6-18. In embodiments, provided is an expression construct comprising an OR coding sequence, wherein the OR coding sequence encodes an amino acid sequence of an OR described in any of Tables 1-5. In embodiments, provided is an expression construct comprising an OR coding sequence, wherein the OR coding sequence encodes an amino acid sequence with greater than 85% identity (e.g., greater than 90%, greater than 95%, greater than 96%, greater than 97%, greater than 98%, or greater than 99% identity) to the amino acid sequences of any of the ORs described in any of Tables 1-5. In embodiments, the expression construct comprises a nucleotide sequence selected from SEQ ID NO: 41-80, or a nucleotide sequence having greater than 85%, greater than 90%, greater than 95%, greater than 96%, greater than 97%, greater than 98%, or greater than 99% sequence identity to a nucleotide sequence selected from SEQ ID NO: 41-80.

In embodiments, the expression construct further comprises an enhancer operably linked to the OR coding sequence. In embodiments, the enhancer is a singular gene choice enhancer operably linked to the OR coding sequence. In aspects, the expression construct is for preferentially expressing in a population of OSNs an OR described herein and for the uses described herein. In some embodiments, the enhancer comprises at least four sequential repeats of a 21 bp sequence wherein each 21 bp sequential repeat comprises the sequence of AACTTTTTAATGA (SEQ ID NO: 81). In some embodiments, the enhancer comprises at least four sequential repeats of ACATAACTTTTTAATGAGTCT (SEQ ID NO: 82). In some embodiments, the enhancer comprises ten or fewer of the 21 bp sequential repeats. In some embodiments, the singular gene choice enhancer sequence comprises ten or fewer of the 21 bp sequential repeats. In some embodiments, the enhancer comprises one or more TetO sequences. In some embodiments, the enhancer is a singular gene choice enhancer. In some embodiments, the expression construct further comprises a nucleic acid sequence encoding a tTA or an rtTA protein. In some embodiments, the rTA or rtTA protein comprises a sequence derived from VP16, VP32, VP48, VP64, and/or GAL4-VP16. In some embodiments, the one or more TetO sequences are located upstream of a cytomegalovirus (CMV) promoter, such as a minimal CMV promoter.

In one aspect, provided is a method for detecting one or more odorants, the method comprising: (a) obtaining a sample from the subject; (b) exposing a biosensor disclosed herein to the sample or to an extract from the sample; and (c) measuring the activation or lack of activation of the one or more ORs, described herein by one or more odorant molecules in the sample obtained from said subject. In embodiments, the levels of the one or more odorants is associated with the levels of one or more neurotransmitters, or a change in the levels of one or more neurotransmitters (e.g., as compared to the control levels for the one or more neurotransmitters) in the CNS of a subject.

In one aspect, provided is a method for detecting a change in the levels of one or more odorants in a sample (e.g., as compared to the control levels for the one or more odorants), the method comprising: (a) obtaining a sample comprising the one or more odorants; (b) exposing a biosensor disclosed herein to the sample or to an extract from the sample; and (c) measuring the activation or lack of activation of the one or more ORs, described herein by one or more odorant molecules in the sample. In some embodiments, the change in the levels of the one or more odorants is associated with a change in the levels of one or more neurotransmitters in the CNS of a subject from which the sample was derived.

In some embodiments of the method, one or more of the neurotransmitters are catecholamines. In some embodiments, the one or more neurotransmitters are selected from the group consisting of dopamine, norepinephrine (noradrenaline), epinephrine (adrenaline), histamine, and/or serotonin. In embodiments, the one or more neurotransmitters are selected from the group of dopamine and serotonin. Thus, in embodiments, the methods disclosed herein can be used to determine if a subject has a change in the levels of one or more neurotransmitters in the CNS, including for example, if the subject has dopamine deficiency. In embodiments, the methods can be used to determine if a subject has, or is likely to develop, a disease associated with dopamine deficiency including PD, depression, schizophrenia, dystonia, or restless leg syndrome. In embodiments, the methods can be used to determine if a subject has, or is likely to develop, PD.

In some embodiments, measuring the activation of the OR comprises detecting a decrease in ATP levels or a change in action potential. In some embodiments, measuring the lack of activation of the OR comprises detecting a lack of decrease in ATP levels or a lack of change in action potential. In some embodiments, measuring the activation of the OR comprises detecting an increase in Ca²⁺, guanosine diphosphate (GDP) and/or cyclic adenosine monophosphate (cAMP) levels. In some embodiments, measuring the lack of activation of the OR comprises detecting a lack of increase in Ca²⁺, GDP and/or cAMP level.

In some embodiments, the one or more odorants associated with a change in the levels of one or more neurotransmitters in the CNS of a subject are present at elevated levels in the sample from the subject as compared to control levels. In some embodiments, the one or more odorants associated with a change in the levels of one or more neurotransmitters in the CNS of a subject are present at reduced levels in the sample from the subject as compared to control levels.

In some embodiments, the subject has a disease associated with a change in the levels of one or more neurotransmitters in the subject's CNS (e.g., as compared to control levels of the one or more neurotransmitters). In some embodiments, the subject has a disease or condition associated with a dopamine deficiency in the CNS. In some embodiments, the disease or condition associated with dopamine deficiency is PD, depression, schizophrenia, dystonia, or restless leg syndrome. In some embodiments, the disease or condition associated with dopamine deficiency is PD. In some embodiments, the levels of the one or more odorants, or the change in the levels of the one or more odorants is associated with the subject having a neurodegenerative disease, such as PD. In embodiments, the sample comprising the one or more odorants comprises sebum, saliva, blood, and/or urine of a subject.

In embodiments, the control levels for the one or more odorants are the levels of the one or more odorants in a sample from one or more individuals that do not suffer from a disease associated with a change in the levels of the one or more odorants. In embodiments, the control levels for the one or more odorants are the levels of the one or more odorants in a sample from one or more individuals that do not suffer from a disease associated with a change in the levels of the one or more neurotransmitters as described herein. In embodiments, the control levels for the one or more odorants are the levels of the one or more odorants in a sample from one or more individuals that do not suffer from PD, depression, schizophrenia, dystonia, and/or restless leg syndrome. In embodiments, the control levels for the one or more odorants are the levels of the one or more odorants in a sample obtained from the subject at a different time. In some embodiments, the control levels for the one or more odorants are obtained from the same subject at an earlier time. In some embodiments, the control levels for the one or more odorants are obtained from the same subject at a later time.

In embodiments, the control levels for the one or more neurotransmitters are the levels of the one or more neurotransmitters in the CNS in one or more individuals that do not suffer from a disease associated with a change in the levels of the one or more neurotransmitters. In some embodiments, the control levels for the one or more neurotransmitters are the levels of the neurotransmitter in the CNS of one or more individuals who do not suffer from a disease associated with a change in the level of dopamine (e.g., a decrease in dopamine) in the CNS. In some embodiments, the control level of a neurotransmitter is the level of that neurotransmitter in the CNS of one or more individuals who do not suffer from a disease associated with a change in the level of serotonin in the CNS. In some embodiments, the control level of a neurotransmitter is the level of that neurotransmitter in the CNS of one or more healthy individuals. In some embodiments, the control level of a neurotransmitter is the level of that neurotransmitter in the CNS of one or more individuals who do not suffer from PD, depression, schizophrenia, dystonia, and/or restless leg syndrome. In some embodiments, the control levels for the one or more neurotransmitters are the levels of the one or more neurotransmitters in the CNS of the subject obtained at a different time. In some embodiments, the control levels for the one or more neurotransmitters are obtained from the same subject at an earlier time. In some embodiments, the control levels for the one or more neurotransmitters are obtained from the same subject at a later time. In some embodiments, the neurotransmitter is a catecholamine. In some embodiments, the neurotransmitter is selected from the group consisting of dopamine, norepinephrine (noradrenaline), epinephrine (adrenaline), histamine, and serotonin. In some embodiments, the neurotransmitter is dopamine or serotonin.

### BRIEF DESCRIPTION OF THE FIGURES

FIG. 1. Schematic of a method for making a biosensor according to the present disclosure and detecting one or more odorants associated with the levels of one or more neurotransmitters in the CNS of a subject and/or associated with a change in the levels of one or more neurotransmitters in the CNS of a subject. A mammal, here a mouse, is engineered to preferentially express an OR disclosed herein in its olfactory sensory neurons (OSNs), and the OSNs, or cilia derived therefrom, are obtained and attached to a chip. The chip may contain additional OSNs, or cilia derived therefrom, derived from mice engineered to preferentially express a different OR in its OSNs. Activation of the responsive ORs, in response to exposure to odorants of interest, may be detected using an optical marker.

FIG. 2. Schematic of the two independent cohorts of patients and healthy volunteers used in the “Deorphanization of Receptors based on Expression Alterations of mRNA levels” (DREAM) experiments. PD cohort 1 (PD1) consisted of ten PD patients and ten age matched healthy volunteers. PD cohort 2 (PD2) consisted in twenty PD patients and twenty age matched healthy volunteers.

FIG. 3. Experimental protocol for the DREAM odor assay with Lewis rats. Wild type (WT) rats were habituated to blank start odor for 48 hours. Following habituation, the rats were split into 2 groups of equal numbers with n=4 and n=24 respectively in PD1 and PD2 (shown in FIG. 1), and exposed to either the pooled gauzes of PD patients in the PD group of rats or the pooled gauzes of Healthy Volunteers (HV) for the HV group of rats. After 5 hours of odor exposure, the rats were sacrificed and total RNA was extracted from the olfactory epithelial (OE) tissue. Subsequent RNAseq deep sequencing or targeted sequencing (see FIG. 2) of an olfactory cDNA library, corresponding to each animal, provided data for differential gene expression analysis by comparing the normalized read counts. BL=plain shirt, PDS=shirt exposed to sebum from patient with PD, PDUA=shirt exposed to under arm area of patient with PD.

FIG. 4. Upset plot summarizing the commonly found (intersection) significantly differentially expressed OR genes between the two cohorts and between the different sequencing and analysis paradigms. Red denotes the OR genes found to be differentially expressed in both the PD1 and the PD2 cohort.

FIG. 5A. Boxplot of the normalized count distributions from DESeq2 analysis in the PD1 cohort for the five rat patch genes (Olr836, Olr837, Olr838, Olr839, Olr840, Olr841) split by group, highlighting the difference of counts between the PD versus the HV groups especially in Olr836 and Olr841. FIG. 5B. Boxplot of the normalized count distributions from DESeq2 analysis in the PD1 cohort for the OR genes found differentially expressed between PD versus HV groups in the PD1 cohort through the read count analysis (Olr292, Olr661, Olr749, Olr804, Olr1185, Olr1558, Olr1160). FIG. 5C. Boxplot of the differential expression from DESeq2 analysis in the PD1 cohort for Olr607 found differentially expressed between PD versus HV groups in the PD1 cohort through a secondary analysis. FIG. 5D. Boxplot of the differential expression from DESeq2 analysis in the PD1 cohort for Olr712 found differentially expressed between PD versus HV groups in the PD1 cohort through a secondary analysis. FIG. 5E. Boxplot of the differential expression from DESeq2 analysis in the PD1 cohort for Olr1381 found differentially expressed between PD versus HV groups in the PD1 cohort through a secondary analysis.

FIG. 6. Volcano plot from one of the DESeq2 analysis of the PD1 cohort showing the significant differential expression of several OR genes with normalized read counts between the PD and HV groups, highlighting Olr836 and Olr841, members of the patch gene family. Genes showing a low transcription signal from the RNAseq normalized counts were filtered.

FIG. 7. A schematic showing the cloning strategy and generation of mice that preferentially express the ORs disclosed herein. SEQ ID TAATGA (SEQ ID NO:84), which is part of the gene choice enhancer sequence (SEQ ID NO:82), is highlighted.

### DETAILED DESCRIPTION

Provided herein are biosensors comprising one or more populations of olfactory neurons, or cilia derived therefrom, that preferentially express certain ORs; biosensors comprising a cell or a population of cells engineered to express certain ORs; biosensors comprising certain isolated ORs; transgenic animals and tissues derived therefrom that preferentially express certain ORs; isolated cells or populations of cells engineered to express certain ORs; expression constructs for the preferential expression of certain ORs; and methods of using the biosensors, transgenic animals, tissues, cells, population of cells, and expression constructs disclosed herein.

Provided herein are methods for detecting one or more odorants associated with the levels of one or more neurotransmitters, or the change in the level of one or more neurotransmitters, in the CNS of a subject. For example, dopamine deficiency in the brain can lead changes in bodily odors or odorant secretions. Accordingly, the present application provides biosensors and methods for using such biosensors for the detection of one or more odorants associated with a disease or condition characterized by altered levels, or change in the levels, of one or more neurotransmitters (including, but not limited to dopamine) in the CNS of a patient.

ORs are amongst the most sensitive and specific chemical detectors in nature, and the discriminatory power of the olfactory system rivals that of any other sensory system. Mammals detect odors using millions of ORs expressed by OSNs that line the nasal cavity. In mammals, olfactory perception of odorants occurs by the combinatorial activation of ORs. OR genes form a large multigene family with about 1,000 members in rodents and about 350 members in humans. The main olfactory epithelium (MOE) expresses ORs through a singular gene choice mechanism whereby only one OR gene allele is expressed in each OSN. Thus, the olfactory sheet is a broad chemical detector, in which each OR is equally distributed in the main olfactory epithelium and only expressed in a small percentage of OSNs—about 0.1% of all OSNs in rodents.

The present disclosure addresses the limitations of current methods for an early and reliable diagnosis of patients with changed levels of one or more neurotransmitters in the CNS by utilizing a cohort of ORs that show activation or lack of activation in presence of one or more odorants in a sample from a patient that exhibits a change in the levels of one or more neurotransmitters in the patient's CNS (e.g., as compared to the control levels for the one or more neurotransmitters).

Provided herein are ORs responsive to odorant that are associated with the levels of one or more neurotransmitters in the CNS of a subject. Provided herein are ORs responsive to odorants that are associated with a change in the levels of one or more neurotransmitters in the CNS of a subject. Provided herein are odorants responsive to odorants associated with a change in the levels of one or more neurotransmitters in the CNS of a subject (e.g., as compared to the control levels for the one or more neurotransmitters).

The ORs described herein are activated by one or more odorants present in a sample from a subject and/or present in a control sample. In embodiments, the odorant is present at elevated levels in the sample from a subject leading to increased activation of the responsive OR. In embodiments, other odorants are present at reduced levels in the sample from the subject and at elevated levels in control samples leading to reduced activation in the responsive OR to the sample from the subject. In embodiments, the ORs described herein are activated by one or more odorants the levels of which are associated with the levels of one or more neurotransmitters in the CNS of a subject or a control. The one or more neurotransmitters can include, for example, dopamine, norepinephrine (noradrenaline), epinephrine (adrenaline), histamine, and/or serotonin. In embodiments, the ORs described herein are activated by one or more odorants the levels of which are associated with a disease characterized by a change in the levels of one or more neurotransmitters in the CNS (e.g., as compared to control levels of the one or more neurotransmitters), including, e.g., a disease associated with a dopamine deficiency in the CNS such as PD, depression, schizophrenia, dystonia, or restless leg syndrome. In embodiments, the ORs described herein are activated by one or more odorants, the levels of which are associated with a neurodegenerative disease in the subject. In embodiments, the neurodegenerative disease is PD.

Provided herein is an OR, wherein the level of activation or lack of activation of the OR is associated with the levels of one or more neurotransmitters in the CNS of a subject. Provided herein is an OR, wherein the activation or lack of activation of the OR is associated with a change in the levels of one or more neurotransmitters in the CNS of a subject. In some embodiments, the OR shows activation, or increased activation, upon exposure to a sample from a subject or to an extract from the sample and shows lack of activation, or reduced activation, upon exposure to a sample from a control or to an extract from the sample. In some embodiments, the OR shows lack of activation, or reduced activation, upon exposure to a sample from a subject or to an extract from the sample and shows activation, or increased activation, upon exposure to a sample from a control or to an extract from the sample. In some embodiments, the OR is capable of being activated by an odorant molecule that is present in a sample from a subject and not present, or present at reduced levels, in the sample of a control. In some embodiments, the OR is capable of being activated by an odorant molecule that is present in a sample from a control and not present, or present at reduced levels, in the sample of a subject. In some embodiments, the OR binds to an odorant present in a sample from a subject and not present, or present at reduced levels, in a sample from a control. In some embodiments, the OR binds to an odorant present in a sample from a control and not present, or present at reduced levels, in a sample from a subject.

In some embodiments, the ORs show activation upon exposure to an odorant, wherein the odorant is present in a sample of a subject and wherein the odorant is present in a sample of a control, but wherein the odorant is present at a different level in the sample of the subject and in the sample of the control. In some embodiments, the level of the odorant in the sample of the subject is higher than the level of the odorant in the sample of the control. In some embodiments, the level of the odorant in the sample of the subject is lower than the level of the odorant in the sample of the control.

ORs useful for the biosensors, transgenic animals, tissues, cells, population of cells, and expression constructs disclosed herein and methods disclosed herein include, but are not limited to, rat ORs Olr836, Olr837, Olr838, Olr839, Olr840, Olr841 (and paralogs), Olr300, Olr1396, Olr292, Olr804, Olr661, Olr749, Olr1185, Olr1381, Olr1558, Olr607, Olr712, 041160. In embodiments, the OR used in the biosensors, transgenic animals, tissues, cells, population of cells, and expression constructs disclosed herein and methods disclosed herein comprises an amino acid sequence selected from SEQ ID NOs: 1-40. In embodiments, the OR comprises an amino acid sequence with greater than 85%, greater than 90%, greater than 95%, greater than 96%, greater than 97%, greater than 98%, or greater than 99% identity to any one of SEQ ID NOs: 1-40. In embodiments, the OR used in the biosensors, transgenic animals, tissues, cells, population of cells, and expression constructs disclosed herein and methods disclosed herein comprises an amino acid sequence selected from SEQ ID NOs: 1 and 6-18. In embodiments, the OR comprises an amino acid sequence with greater than 85%, greater than 90%, greater than 95%, greater than 96%, greater than 97%, greater than 98%, or greater than 99% identity to any one of SEQ ID NOs: 1 or 6-18. In embodiments, the OR used in the biosensors, transgenic animals, tissues, cells, population of cells, and expression constructs disclosed herein and methods disclosed herein comprises an amino acid sequence of an OR described in any of Tables 1-5. In embodiments, the OR comprises an amino acid sequence with greater than 85%, greater than 90%, greater than 95%, greater than 96%, greater than 97%, greater than 98%, or greater than 99% identity to an amino acid sequence of an OR described in any of Tables 1-5.

In embodiments, the OR comprises an amino acid sequence of an OR described in Table 1. In embodiments, the OR comprises an amino acid sequence with greater than 85%, greater than 90%, greater than 95%, greater than 96%, greater than 97%, greater than 98%, or greater than 99% identity to an amino acid sequence of an OR described in Table 1. In embodiments, the OR comprises an amino acid sequence of an OR described in Table 2. In embodiments, the OR comprises an amino acid sequence with greater than 85%, greater than 90%, greater than 95%, greater than 96%, greater than 97%, greater than 98%, or greater than 99% identity to an amino acid sequence of an OR described Table 2. In embodiments, the OR comprises an amino acid sequence of an OR described in Table 3. In embodiments, the OR comprises an amino acid sequence with greater than 85%, greater than 90%, greater than 95%, greater than 96%, greater than 97%, greater than 98%, or greater than 99% identity to an amino acid sequence of an OR described in Table 3. In embodiments, the OR comprises an amino acid sequence of an OR described in Table 4. In embodiments, the OR comprises an amino acid sequence with greater than 85%, greater than 90%, greater than 95%, greater than 96%, greater than 97%, greater than 98%, or greater than 99% identity to an amino acid sequence of an OR described in Table 4. In embodiments, the OR comprises an amino acid sequence of an OR described in Table 5. In embodiments, the OR comprises an amino acid sequence with greater than 85%, greater than 90%, greater than 95%, greater than 96%, greater than 97%, greater than 98%, or greater than 99% identity to an amino acid sequence of an OR described in Table 5.

In some embodiments, the OR is encoded by a patch gene. Patch genes are a group of genes encoding OR proteins expressed in a localized region, a “patch”, of the olfactory epithelium. The patch domain of expression for this group of ORs has been well described for mouse genes, but is also conserved amongst other terrestrial mammals. Patch OR genes tend to be linked and show high degree of sequence homology amongst paralogues in the same species and orthologues across species. A subset of patch OR genes, respond to sebum derived odors including long chain aldehydes such as C14-C18. In mice, trace amine-associated receptor (TAAR) chemosensory genes are expressed in a domain and respond to amines, whereas Class I OR genes are expressed in a different domain and respond to acids. As described herein, certain patch ORs preferentially bind to sebum related odors.

Also useful for the biosensors, transgenic animals, tissues, cells, population of cells, and expression constructs disclosed herein and methods disclosed herein are ORs that are homologs of any one of the ORs disclosed herein. In some embodiments, the OR is an orthologue or a paralogue of an OR disclosed herein. As used in this specification, a homolog of an OR is an OR that shares 85% or more homology (amino acid identity plus amino acid similarity) as compared to an OR. As used in this specification, an orthologue of an OR is an OR (i) that is encoded by a gene that is located at an orthologous position in the genome as compared to the OR gene or that is encoded by a gene that exhibits synteny with the OR gene and (ii) that exhibits greater than 85% protein homology (amino acid identity plus amino acid similarity) as compared to the OR. As used herein, a paralogue is a gene that shares a high degree of homology to another gene due to a gene duplication event.

Once an OR has been identified in, for example, in a rat or a mouse, a person of ordinary skill in the art can readily identify homologous ORs derived from other species and can verify that these homologous ORs serve the same or a very similar function. Methods for identifying homologous proteins are well known in the art (see for example Pearson W R, An introduction to sequence similarity (“homology”) searching. Curr Protoc Bioinformatics. 2013 June; Chapter 3: Unit 3.1, incorporated herein by reference). Thus, provided herein are for example, rat, mouse, or other mammalian ORs that are homologs or orthologs to the ORs identified herein. A non-exhaustive, non-limiting list of orthologues and paralogues for the ORs identified in this disclosure can be found in Tables 2-5.

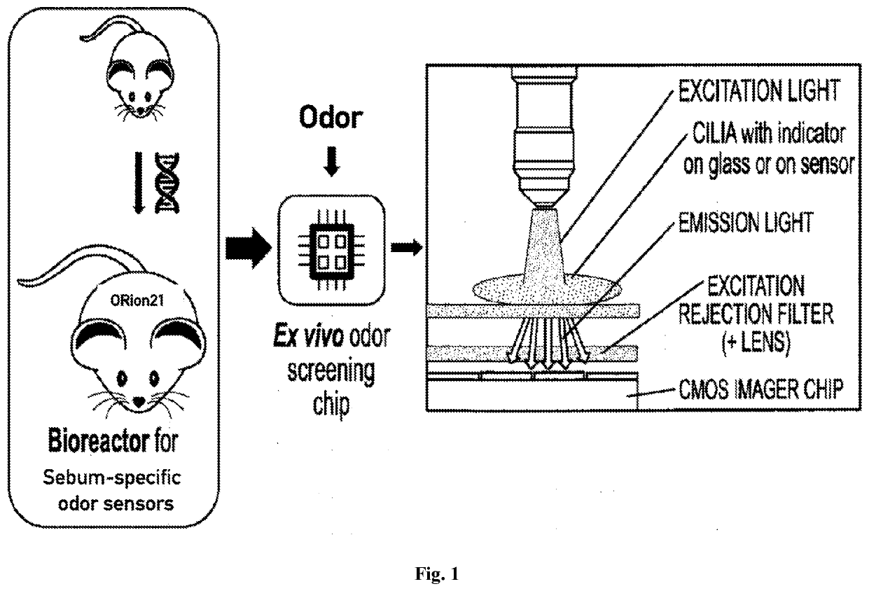

As used herein, a “biosensor” is an analytical device or system which may be used to detect, quantitatively or qualitatively, the presence, absence, and/or concentration of a biological analyte (such as an odorant molecule) in a sample. In some embodiments, the biosensor converts a biological response into a signal that can be detected, for example an electrical signal or light signal. In some embodiments, the biosensor comprises a recognition element (e.g., an OR described herein), which can recognize or capture a specific analyte, and a transducer, which transmits the presence or absence of an analyte into a detectable signal. In some embodiments, the biosensor comprises a chip or is utilized as part of a biochemical assay. A schematic of a method for making a biosensor according to the present disclosure and detecting one or more odorants is shown in FIG. 1.

In embodiments, the biosensors described herein are for use in detecting one or more odorants, or a change in the levels of one or more odorants (e.g., as compared to control levels of the one or more odorants), in a sample from the subject. In embodiments, the one or more odorants, or the change in the levels of the one or more odorants, is associated with the levels of one or more neurotransmitters, or a change in the levels of one or more neurotransmitters (e.g., as compared to control levels of the one or more neurotransmitters), in the CNS of a subject. Thus, in embodiments, the biosensors described herein are for use in detecting the levels of one or more neurotransmitters, or a change in the levels of one or more neurotransmitters in the CNS of a subject. The one or more neurotransmitters can include, for example, dopamine, norepinephrine (noradrenaline), epinephrine (adrenaline), histamine, and/or serotonin.

In embodiments, the biosensors described herein are for use in detecting one or more odorants, or the change in the levels of the one or more odorants, associated with a disease characterized by a change in the levels of one or more neurotransmitters in the CNS (e.g., as compared to control levels of the one or more neurotransmitters), including, e.g., a disease associated with a dopamine deficiency in the CNS such as PD, depression, schizophrenia, dystonia, or restless leg syndrome. Thus, in embodiments, the biosensors described herein are for use in detecting a disease characterized by a change in the levels of one or more neurotransmitters in the CNS including, e.g., a disease associated with a dopamine deficiency in the CNS such as PD, depression, schizophrenia, dystonia, or restless leg syndrome. In embodiments, the one or more odorants, or the change in the levels of the one or more odorants, detected by the biosensors disclosed herein, are associated with a neurodegenerative disease in the subject. Thus, in embodiments, the biosensors described herein are for use in detecting a neurodegenerative disease in the subject. In embodiments, the neurodegenerative disease is PD.

In one aspect, provided is a biosensor comprising a cell or population of cells engineered to express an OR described herein. In such embodiments, the cell may be an isolated eukaryotic cell or the population of cells may be a population of isolated eukaryotic cells. In some embodiments, the biosensor comprises a eukaryotic cell or a population of eukaryotic cells genetically engineered to express an OR described herein. As used herein, the terms “genetically engineered,” “genetically modified,” or “engineered” are used interchangeably and refers to any human or human-caused manipulation intended to introduce a genetic change in a cell or organism. Such manipulation includes altering the genetic material (such as DNA or RNA) existing in a cell or in an organism or introducing exogenous genetic material into a cell or into an organism.

Genetic alterations include, for example, a gene deletion or some other functional disruption of the genetic material. Genetic alterations also include modifications that introduce expressible nucleic acids encoding polypeptides (including, but not limited to ORs). In some embodiments, the genetic alteration restores, corrects, or modifies expression of a gene. In some embodiments, genetic alteration includes the introduction of a regulatory element (including, but not limited to, an enhancer, silencer, promoter, or other transcriptional regulator) that affects the expression of a naturally present gene.

The nucleic acid introduced into the cell or into the organism can originate from any species. In some embodiments, the nucleic acid sequence introduced into the cell or into the organism is derived from the same species or a different species. Alternatively, the nucleic acid sequence introduced into the cell or into the organism might not occur anywhere in nature and may be created by the chemical synthesis of nucleic acid. Accordingly, “genetically engineered” may refer to a cell or an organism that contains one or more artificial or recombinant sequences of nucleotides which have been created through molecular cloning techniques to bring together genetic material that is not natively found together.

The genetic alteration of the cell or organism may be achieved by a variety of techniques, including, but not limited to, calcium-phosphate-mediated transfection, diethylaminoethyl (DEAE)-mediated transfection, microinjection, viral transformation, protoplast fusion, lipofection, and/or the use of meganucleases and zinc finger nucleases, transcription activator-like effector nucleases (TALENs) or a Cas9-guideRNA system (adapted from Clustered Regularly Interspaced Short Palindromic Repeats (CRISPR)).

The genetically altered cell or organism may contain and/or express the foreign nucleic acid (e.g., an OR coding sequence) in either a transient or long-term manner. In general, transient expression occurs when foreign DNA does not stably integrate into the chromosomal DNA of the transfected cell or organism. In contrast, long-term expression of foreign DNA occurs when the foreign DNA has been stably integrated into the chromosomal DNA of the transfected cell or organism.

Not all eukaryotic cells in a population that is genetically engineered to express a polypeptide (such as an OR) or that is genetically engineered to change the expression level of a polypeptide (such as an OR) will express the polypeptide (such as an OR) in a significant amount. In some embodiments, at least 50%, at least 60%, at least 70%, at least 80%, or at least 90% of the eukaryotic cells genetically engineered to express a polypeptide (such as an OR) will express the polypeptide (such as an OR) at a given time.

In some embodiments, the biosensor comprises a eukaryotic cell or a population of eukaryotic cells, wherein the eukaryotic cell is an OSN and the population of eukaryotic cells is a population of OSNs. OSNs can be obtained from transgenic animals such as the transgenic animals described herein by methods known in the art.

In some embodiments, the biosensor comprises a eukaryotic cell or a population of eukaryotic cells, wherein the eukaryotic cell is yeast cell and the population of eukaryotic cells is a population of yeast cells, HEK cells, or CHO cells.

In some embodiments, the biosensor comprises a eukaryotic cell other than an OSN that expresses an OR disclosed herein. In some embodiments, the OR may be fused to a processing/transport segment that directs the processing and transport of the OR to the cell membrane of the host cell. In some embodiments, the biosensor comprises a eukaryotic cell other than an OSN that expresses the hypervariable segment of an OR, wherein such segment contains at least one odorant binding site of the OR. Methods for the expression of ORs and detection of OR activation in yeast have been described in U.S. Pat. No. 7,223,550 and Patent Application No. PCT/2017/019179, both of which are incorporated herein by reference.

In embodiments, the biosensor comprises one or more populations of eukaryotic cells wherein each population of eukaryotic cells preferentially expresses an OR described herein. In embodiments, the biosensor comprises one or more populations of OSNs, wherein each population preferentially expresses a different OR described herein. In some embodiments, the biosensor comprises at least two, at least three, at least four, or at least five populations of eukaryotic cells (e.g., OSNs), wherein each population preferentially expresses a different OR described herein. In embodiments, the biosensor comprises 1, 2, 3, 4, 5, 6, 7, 8, 9, 10, 11, 12, 13, 14, 15 or more cell populations, wherein each population preferentially expresses a different OR described herein. In embodiments, the preferentially expressed OR comprises (1) an amino acid sequence selected from SEQ ID NOs: 1-40; (2) an amino acid sequence with at least 85%, at least 90%, at least 95%, at least 98, or at least 99% identity to any one of SEQ ID NOs: 1-40; (3) an amino acid sequence selected from SEQ ID NOs: 1 and 6-18; (4) an amino acid sequence with at least 85%, at least 90%, at least 95%, at least 98, or at least 99% identity to any one of SEQ ID NOs: 1 or 6-18; (5) an amino acid sequence of an OR described in any of Tables 1-5; and/or (6) an amino acid sequence with at least 85%, at least 90%, at least 95%, at least 98, or at least 99% identity to an amino acid sequence of an OR described in any of Tables 1-5.

In a non-limiting example, the biosensor comprises two populations of cells with each population preferentially expressing a different OR comprising (1) an amino acid sequence selected from SEQ ID NOs: 1-40; (2) an amino acid sequence with at least 85%, at least 90%, at least 95%, at least 98, or at least 99% identity to any one of SEQ ID NOs: 1-40; (3) an amino acid sequence selected from SEQ ID NOs: 1 and 6-18; (4) an amino acid sequence with at least 85%, at least 90%, at least 95%, at least 98, or at least 99% identity to any one of SEQ ID NOs: 1 or 6-18; (5) an amino acid sequence of an OR described in any of Tables 1-5; and/or (6) an amino acid sequence with at least 85%, at least 90%, at least 95%, at least 98, or at least 99% identity to an amino acid sequence of an OR described in any of Tables 1-5. In another non-limiting example, the biosensor comprises three, four, five, six, or more populations of cells with each population preferentially expressing a different OR comprising (1) an amino acid sequence selected from SEQ ID NOs: 1-40; (2) an amino acid sequence with at least 85%, at least 90%, at least 95%, at least 98, or at least 99% identity to any one of SEQ ID NOs: 1-40; (3) an amino acid sequence selected from SEQ ID NOs: 1 or 6-18; (4) an amino acid sequence with at least 85%, at least 90%, at least 95%, at least 98, or at least 99% identity to any one of SEQ ID NOs: 1 or 6-18; (5) an amino acid sequence of an OR described in any of Tables 1-5; and/or (6) an amino acid sequence with at least 85%, at least 90%, at least 95%, at least 98, or at least 99% identity to an amino acid sequence of an OR described in any of Tables 1-5.

As used herein, “preferential expression” or “preferentially express” refers to an increase in the number of cells in a population of cells that express a specific OR as compared to the wild type or unmodified population of cells. For example, in the case of Rattus norvegicus, the preferential expression of an OR is compared to the expression of other Rattus norvegicus ORs. In the case of a Mus musculus OR, expression of the OR is compared to the expression of other Mus musculus ORs. For example, in the mouse, a typical OR is expressed in about 10,000 OSNs out of approximately 10,000,000 OSNs. By cloning a suitable enhancer upstream of an OR gene, that OR can be preferentially expressed in an increased number of OSNs, for example 500,000 to 2 million neurons. In embodiments, the percentage of cells in a population of cells that expresses an OR described herein is greater than 5%, greater than 10%, greater than 20%, greater than 30%, greater than 40%, greater than 50%, or greater than 60%. In embodiments, the percentage of cells in a population of cells that expresses the OR is between 10 and 90%.

In one embodiment, the methods described in International Patent Publication WO2017/024028, said methods are hereby incorporated by reference, are used in conjunction with a sequence encoding an OR, including the disclosed OR coding sequences SEQ ID NOs: 41-80 or OR polynucleotide sequences encoding the disclosed amino acid sequences SEQ ID NOS: 1-40. WO2017/024028 describes OR expression constructs, vectors and methods for producing genetically modified non-human vertebrates that preferentially express a selected OR in the OSNs, said expression constructs and methods are incorporated herein by reference (see FIG. 1 of WO2017/024028).

Accordingly, in some embodiments, provided is a nucleic acid construct for the preferential expression of an OR described herein, wherein the nucleic acid construct comprises an enhancer operably linked to the sequence encoding the preferentially expressed OR. In embodiments, the enhancer is a singular gene choice enhancer. In some embodiments, the enhancer comprises at least three, at least four, at least five, at least six, at least seven, at least eight, at least nine, or at least ten sequential repeats of a 21 base pair (bp) sequence wherein each 21 bp sequential repeat comprises the sequence AACTTTTTAATGA (SEQ ID NO: 81). In some embodiments, the enhancer comprises at least three, at least four, at least five, at least six, at least seven, at least eight, at least nine, or at least ten sequential repeats of ACATAACTTTTTAATGAGTCT (SEQ ID NO: 82). In embodiments, the enhancer comprises three, four, five, six, seven, eight, nine, ten, or more sequential repeats each containing YTTTTAATGAR where Y=C/T and R=A/G (SEQ ID NO: 83). In some embodiments, the enhancer comprises ten or fewer of the 21 bp sequential repeats. In embodiments, the enhancer comprises four to ten (e.g., four, five, six, seven, eight, nine, or ten) of the 21 bp sequential repeats described herein.

In one aspect, provided are compositions and methods for preferentially expressing an OR using a tetracycline-controlled transactivator protein in combination with a TetR DNA binding site (TetO sequence). Together, these regulatory DNA sequence control expression of an OR in response to the presence or absence of tetracycline (Tc) or Tc derivatives including, but not limited to, doxycycline (Dox).

In embodiments, the tetracycline-controlled transcriptional transactivator protein is tTA, which is composed of the Tet repressor DNA binding protein (TetR) from the Tc resistance operon of Escherichia coli transposon Tn10 fused to the transactivating domain of VP16 from Herpes simplex virus. In the absence of Tc or Dox, tTA binds to TetO and activates transcription of the target gene. In the presence of Tc or Dox, tTA cannot bind to TetO, and expression from the target gene remains inactive. In one embodiment, one or more repeats of the TetO sequence are placed upstream of a minimal promoter. In one embodiment, the minimal promoter is a CMV promoter.

In another embodiment, a reverse tetracycline-controlled transcriptional transactivator called rtTA is used. rtTA is a fusion protein comprised of the TetR repressor and the VP16 transactivation domain; however, a four amino acid change in the tetR DNA binding moiety alters rtTA's binding characteristics such that it can only recognize the TetO sequence in the presence of the Dox effector. Thus, here, transcription of the target gene is stimulated by rtTA only in the presence of Tc or Dox.

In some embodiments, the rTA or rtTA comprise viral structural protein VP32, VP48, or VP64 instead of VP16 as the activator. In some embodiments, the rTA or rtTA comprise GAL4-VP16 as the activator. GAL4-VP16 comprises the DNA-binding fragment of the yeast activator GAL4 and part of VP16, in combination with a promoter containing upstream activating sequences (UAS). In some embodiments, the promoter region further comprises a CMV promoter or fragment thereof.

In embodiments, the biosensors disclosed herein comprise cilia isolated from one or more populations of OSNs that express one or more ORs described herein. In the olfactory system, millions of hair-like olfactory cilia protrude from the dendrites of the OSNs into the mucus of the MOE that lines the nasal cavity. ORs present in the membranes of these cilia signal their activation by odorants through a G protein-mediated signaling cascade in which binding of the odor activates type III adenylate cyclase (ACIII) and causes a rapid rise in levels of cAMP, which binds to cyclic-nucleotide gated channels causing influx of Ca²⁺. There is also evidence that olfactory receptors can signal via G-protein activation of phosphoinositidase C, with subsequent production of inositol 1,4,5-triphosphate and 1,2-diacylglycerol second messengers.

In certain embodiments, the biosensor comprises cilia isolated from a population of OSNs that preferentially expresses an OR disclosed herein. Olfactory cilia can be detached from the main olfactory epithelium thereby providing an ex vivo system amenable to monitor OR activation, as olfactory signal transduction events are exclusively initiated within these cilia. Cilia can be obtained from olfactory epithelial tissue by methods known in the art. For example, Kuhlmann et al., (Molecular & Cellular Proteomics (2014), 13:1828-1843) and Mayer et al., (Proteomics (2009), 9:322-334) provide protocols for isolation of olfactory cilia and those protocols are incorporated herein by reference. Sklar et al. (J. of Biological Chemistry (1986), 261:15538-15543), and Pfeuffer et al. (J. of Biological Chemistry (1989), 264:18803-18807) also provide protocols for isolation of olfactory cilia and those protocols are also incorporated herein by reference. Following isolation, cilia preparations may be stored at −80° C. for months without significant loss in activity.

For example, a portion of the OSN (i.e., olfactory cilia) is extracted as follows: Olfactory epithelia from 6-week old mice are dissected and briefly washed in cold buffer. The tissue is incubated in cold extraction buffer containing calcium for 20 minutes and subsequently spun down for 10 minutes, in which the supernatant is collected for the following steps. This extraction process is repeated once on the tissue. The combined supernatant is collected and spun down at high speed in a cooled ultracentrifuge for 30 minutes. The resulting pellet contains olfactory cilia and is reconstituted in buffer with glycerol and protease inhibitor, aliquoted and snap-frozen with liquid nitrogen, and stored at −80° C. until use.

The methods recited above can be used to isolate cilia from a non-human mammal, for example a rat or mouse, where the population of OSNs in the OE of the mammal preferentially express an OR disclosed herein. The OR can be an endogenous OR where the OR gene has been modified to drive the preferential expression of the OR in the population of OSNs. In other embodiments, the OR is an exogenous OR expressed from a nucleic acid, vector, or construct that drives the preferential expression of the OR in the population of OSNs.

In embodiments, the biosensor is a chip or otherwise involves attachment of populations of cells or cilia to a solid support. Accordingly, the biosensor may comprise (i) an array of individual populations of cells (each population preferentially expressing a different OR disclosed herein), or (ii) an array of individual populations of cilia, where each population of cilia is derived from a population of OSNs, where each population of OSNs preferentially expresses a different OR disclosed herein. Such an array can also be used when the biosensor comprises a multi-well format.

In some embodiments, the biosensor comprises populations of eukaryotic cells disposed on a solid support. In some embodiments, the biosensor comprises populations of OSNs or cilia derived therefrom that were extracted from a transgenic non-human mammal and subsequently disposed on a solid support. Examples of suitable solid supports include, but are not limited to, silicon, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, optical fiber bundles, and a variety of other polymers. Preferably, the solid support allows optical detection and does not appreciably fluoresce. In one embodiment, the surface of the solid support is modified to contain microwells, i.e. depressions in the surface of the solid support. This may be done as is generally known in the art using a variety of techniques, including, but not limited to, photolithography, stamping techniques, pressing, casting, molding, microetching, electrolytic deposition, chemical or physical vapor deposition employing masks or templates, electrochemical machining, laser machining or ablation, electron beam machining or ablation, and conventional machining. As will be appreciated by those in the art, the technique used will depend on the composition and shape of the solid support. In one embodiment, the interior surfaces of the microwells may be coated with a thin film or passivation layer of biologically compatible material. For example, materials known to support cell growth or adhesion may be used, including, but not limited to, agar, fibronectin, any number of known polymers including collagen, polylysine and other polyamino acids, polyethylene glycol and polystyrene, growth factors, hormones, cytokines, etc. In addition, coatings or films of metals such as a metal such as gold, platinum or palladium may be employed. In an alternative embodiment, an indicator compound, for example, a fluorophore, a chromophore or dye, may be attached to the microwell surface for detecting cellular responses to OR activation. In some embodiments, the biosensor further comprises one or more of an electromagnetic radiation source, a detection element, an optical filter, components to deliver or remove fluids, a collection chamber, a cover plate, an electrode, an integrated circuit, and a hydrogel.

In one aspect, provided is a biosensor, wherein the biosensor comprises an isolated OR described herein. In some embodiments, the biosensor comprises a lipid bilayer comprising the OR. In some embodiments, the OR is present in a nanovesicle, nanosome, nanodisc, or is suspended in a lipid bilayer. In some embodiments the biosensor further comprises a marker for detecting activation or lack of activation of the OR, wherein the activation or lack of activation occurs upon exposure of the one or more populations of OSNs to a sample from a subject exhibiting a change in the levels of one or more neurotransmitters in the CNS (e.g., as compared to the control levels the one or more neurotransmitters).

A person skilled in the art will appreciate that the activation or lack of activation of an OR can be measured in various ways. For instance, activation of an OR may be detected by monitoring a decrease in ATP levels or an increase in Ca²⁺, GDP, cAMP, inositol 1,4,5-triphosphate and/or 1,2-diacylglycerol levels using conventional methods. Conversely, lack of activation of an OR may be detected by observing a lack of decrease in ATP levels or a lack of increase in Ca²⁺, GDP, cAMP, inositol 1,4,5-triphosphate and/or 1,2-diacylglycerol levels using conventional methods.

In some embodiments, a marker may be provided to detect the activation (or lack thereof) of an OR upon exposure to a sample from a patient or to an extract from the sample. The use of markers permits the measurement of OR activation (or lack thereof) using conventional methods, including the measurement of fluorescence, luminescence, phosphorescence, visible light, radioactivity, colorimetry, X-ray diffraction or absorption, electricity, change in electric potential, or magnetism. In some embodiments, the marker may be a fluorescent dye. Examples of suitable dyes include calcium-sensitive dyes such as fura-2, fluo-3, fluo-4, fluo-5F, indo-1, and Oregon Green BAPTA. The marker may be integrated into the biosensor using, for example, the techniques described in International Patent Publication WO2017024028, incorporated herein by reference. Marker proteins may be co-expressed with the one or more preferentially expressed ORs. Examples of suitable marker proteins include GECO2.1, GCaMP6f, Flamindo, Flamindo2, and Pink Flamindo.

In some embodiments, the OR is further genetically or chemically modified to allow detection of OR activation by inter- or intra-molecular fluorescence resonance energy transfer (FRET), bioluminescence resonance energy transfer (BRET), or bimolecular fluorescence complementation (BiFC).

The responsiveness of a transgenic mammal that preferentially expresses one or more ORs described herein can be determined using behavioral analysis. For example, the control (e.g., wild type) animal and transgenic animal are compared in terms of reaction to (e.g., time spent near) to a sebum sample derived from a patient as compared to a control sample.

In some embodiments, the activation of the OR is determined in a biochemical assay. In some embodiments, populations of OSNs, or cilia derived therefrom, that express an OR are isolated and the activation of the OR is detected ex vivo. In one embodiment, the cilia of the OSNs are further isolated using a deciliation protocol and used for the detection of activation of the OR.

Provided herein are transgenic animals, tissues, and cells isolated from the transgenic animals, wherein the transgenic animals have been engineered to express one or more ORs described herein. In embodiments, the transgenic animal comprises an olfactory epithelium, wherein the neurons of the OE preferentially express an OR described herein. In some embodiments, the olfactory epithelium is the main olfactory epithelium.

In some embodiments, the transgenic animal is a non-human mammal. In some embodiments, the non-human mammal belongs to the family of Bovidae, Canidae, and Muridae. In some embodiments, the non-human mammal is a rat, mouse, dog, cat, goat, chicken, sheep, pig, or primate.

In one aspect, provided is a transgenic animal comprising an olfactory epithelium in which the OSNs preferentially express an OR disclosed herein. In embodiments, the transgenic animal comprises: (a) a transgene sequence encoding an OR comprising an amino acid sequence selected from the group consisting of (1) an amino acid sequence selected from SEQ ID NOs: 1-40; (2) an amino acid sequence with at least 85%, at least 90%, at least 95%, at least 98, or at least 99% identity to any one of SEQ ID NOs: 1-40; (3) an amino acid sequence selected from SEQ ID NOs: 1 and 6-18; (4) an amino acid sequence with at least 85%, at least 90%, at least 95%, at least 98, or at least 99% identity to any one of SEQ ID NOs: 1 or 6-18; (5) an amino acid sequence of an OR described in any of Tables 1-5, and/or (6) an amino acid sequence with at least 85%, at least 90%, at least 95%, at least 98, or at least 99% identity to an amino acid sequence of an OR described in any of Tables 1-5; and (b) an enhancer operably linked to the transgene sequence. In embodiments, the enhancer is a singular gene choice enhancer described herein.

Also provided herein is a tissue isolated from any of the transgenic animals described herein. In some embodiments, the isolated tissue is an olfactory epithelium. In some embodiments, the isolated tissue is a main olfactory epithelium.

Also provided herein is a cell or a population of cells isolated from any of the transgenic animals described herein. In some embodiments, the isolated cell or a population of cells is an olfactory epithelium cell or a population of olfactory epithelium cells.

A sample from a subject, as used herein, refers to any substance or material obtained from, or derived from, a subject, comprising an odorant that can be detected by one or more of the ORs disclosed herein. A sample, may be obtained from breath, sebum, saliva, blood, urine, sweat, or semen of a subject and materials derived therefrom including, for example plasma, lipids, proteins, and small molecules. A sebum sample, for example, may be obtained from a cotton pad, swab, gauze, bandage, sportive tape, fabric, tissue, adsorbent-coated fiber, absorbent paper, clothing, or other material placed in contact with a subject's skin.

Methods for the extraction of odorants from samples are known in the art. For example, the sample (e.g., a sebum sample) may be collected with a suitable matrix, for example, with a cotton pad, swab, gauze, bandage, sportive tape, fabric, tissue, adsorbent-coated fiber, absorbent paper or clothing worn by the patient. Tape specially designed for the collection of sebaceous lipids is available under the name Sebutape® Adhesive Patches (cat #S100) or Sebutape® Indicator Strips (cat #5232) (CuDerm Corporation, Dallas, TX).

In some embodiments, the one or more odorants are extracted from the sample using an extraction medium. An extraction medium may be composed of polar and/or non-polar organic solvents such as chloroform, methanol, ether, propanol, isopropanol, di-chloromethane, tri-methyl-pentene, hexane, or heptane or their combinations and may contain an aqueous phase with or without modifiers (such as acids or bases). In some embodiments, the odorant is collected using dynamic headspace adsorption onto various porous polymers (e.g., Tenax, Porapak Q). Such methods may be used for collecting airborne odorants. In some embodiments, the odorant is directly collected into an adsorbent trap. In some embodiments, the odorants are collected using solid-phase microextraction (SPME), solvent-assisted flavor evaporation (SAFE), or simultaneous distillation extraction (SDE).

In some embodiments, during the days before collection of the sample(s), the patient is asked to follow particular instructions related to diet and the use of fragrance soap/shampoo. In some embodiments, the patient is asked to avoid spicy food and garlic several days before sampling. In some embodiments, the patient is asked to use no deodorant, no perfume, and to use fragrance-free soaps the days before odor collection. In some embodiments, the patient is instructed to do some exercise so that the skin became sweaty.

Provided herein are methods of using the biosensors, transgenic animals, tissues, and cells (including OSNs and cilia derived therefrom) disclosed herein for detecting one or more odorants. In one aspect, provided is a method of detecting one or more odorant molecules, the method comprising: (a) obtaining a sample from a subject, wherein the sample comprises one or more odorant molecules; (b) exposing one or more populations of eukaryotic cells to the sample obtained from said subject, wherein each population of eukaryotic cells preferentially expresses a set of ORs comprising an amino acid sequence selected from the group consisting of (1) SEQ ID NOs: 1-40, (2) an orthologue or a paralogue of an OR represented by any one of SEQ ID NOs: 1-40, (2) an amino acid sequence with greater than 85% identity to any one of SEQ ID NOs: 1-40, (3) an amino acid sequence selected from SEQ ID NOs: 1 or 6-18, (4) an amino acid sequence with greater than 85% identity to any one of SEQ ID NOs: 1 or 6-18, (5) an amino acid sequence of an OR described in any of Tables 1-5, and/or (6) an amino acid sequence with greater than 85% identity to an amino acid sequence of an OR described in any of Tables 1-5; and (c) measuring in each of the one or more populations of eukaryotic cells the activation or lack of activation of the preferentially expressed set of ORs by the one or more odorant molecules in the sample obtained from said subject. In some embodiments, the one or more populations of eukaryotic cells are one or more populations of OSNs, or cilia derived therefrom. In embodiments, the odorant molecules in the sample are compared to the odorant molecules of a control sample.

The biosensors provided herein are useful for detecting odorants, wherein the odorants are associated with a change in the levels of one or more neurotransmitters in the CNS of a patient (e.g., as compared to the control levels for the one or more neurotransmitters). Accordingly, the biosensors disclosed herein can be used in a method for diagnosing a disease or condition associated with a change in the levels of one or more neurotransmitters in the CNS as compared to the controls levels for the one or more neurotransmitters. In some embodiments, the biosensors is used to make a diagnosis prior to the subject manifesting the clinical symptoms that are used in the diagnosis of the disease. In some embodiments, the biosensors can be used to confirm a previous diagnosis of a disease or condition associated with a change in the levels of one or more neurotransmitters in the CNS.

Additionally, the biosensors disclosed herein can be used in a method for monitoring the disease status of a patient over time, wherein the patient has a disease or condition associated with a change in the levels of one or more neurotransmitters in the CNS as compared to the controls levels for the one or more neurotransmitters, wherein the controls levels for the one or more neurotransmitters were obtained from one or more individuals that do not have the disease or condition.

The biosensors disclosed herein can also be used to identify patients at increased risk of and/or with a predisposition of developing a disease or condition associated with a change in the levels of one or more neurotransmitters in the CNS as compared to the controls levels for the one or more neurotransmitters, wherein the controls levels for the one or more neurotransmitters were obtained from one or more individuals that do not have the disease or condition.

In some embodiments, the patient has a neurotransmitter deficiency in the CNS. In some embodiments, the patient has a dopamine and/or a serotonin deficiency in the CNS. In some embodiments, the patient has PD, depression, schizophrenia, dystonia, and/or restless leg syndrome. In some embodiments, the patient has an increased risk of developing a neurotransmitter deficiency in the CNS. In some embodiments, the patient has an increased risk of developing a dopamine and/or a serotonin deficiency in the CNS. In some embodiments, the patient has an increased risk of developing PD, depression, schizophrenia, dystonia, and/or restless leg syndrome.

As used herein, “subject” or “patient” includes individuals that are exhibiting signs of a change in the levels of one or more neurotransmitters in the CNS as well as individuals that have not yet begun exhibiting symptoms of a change in the levels of one or more neurotransmitters in the CNS.

Accordingly, in one aspect, provided is a biosensor for diagnosing a disease or condition associated with the levels of one or more neurotransmitters or a change in the levels of one or more neurotransmitters in the CNS of a subject, or for identifying an individual with an increased risk of developing diagnosing a disease or condition associated with the levels or a change in the levels of one or more neurotransmitters in the CNS of a subject, wherein the biosensor comprises: one or more populations of OSNs, or cilia derived therefrom, wherein each population of OSNs preferentially expresses an OR. In embodiments, the OR is (1) an OR that comprises an amino acid sequence selected from the group consisting of SEQ ID NOs: 1-40, (2) a orthologue or a paralogue of an OR comprising an amino acid sequence selected from the group consisting of SEQ ID NOs: 1-40, (3) an OR comprising an amino acid sequence with greater than 85% identity to any one of SEQ ID NOs: 1-40, (4) an OR that comprises an amino acid sequence selected from the group consisting of SEQ ID NOs: 1 and 6-18, (5) a orthologue or a paralogue of an OR comprising an amino acid sequence selected from the group consisting of SEQ ID NOs: 1 or 6-18, (6) an OR comprising an amino acid sequence with greater than 85% identity to any one of SEQ ID NOs: 1 or 6-18, (7) an OR comprising an amino acid sequence of an OR described in any of Tables 1-5, and/or (8) an OR comprising an amino acid sequence with greater than 85% identity to an amino acid sequence of an OR described in any of Tables 1-5.

Thus, in embodiments, the biosensor comprises one or more populations of OSNs, or cilia derived therefrom, wherein the one or more populations of OSNs comprises at least a first population that preferentially expresses a first amino acid sequence and a second population that preferentially expresses a second amino acid sequence, wherein the first amino acid sequence and the second amino acid sequence are different and are independently selected from the OR sequences described herein, including for example, a group consisting of SEQ ID NOs: 1-40, an orthologue or a paralogue of an OR represented by any one of SEQ ID NOs: 1-40, an amino acid sequence with greater than 85% identity to any one of SEQ ID NOs: 1-40, an amino acid sequence described in any of Tables 1-5, and/or an amino acid sequence with greater than 85% identity to an amino acid sequence described in any of Tables 1-5.

Also provided is a method of (i) diagnosing a disease or condition associated with the levels of one or more neurotransmitters or a change in the levels of one or more neurotransmitters in the CNS of a subject and/or (ii) identifying an increased risk of developing a disease or condition associated with the levels of one or more neurotransmitters or a change in the levels of one or more neurotransmitters in the CNS, the method comprising: (a) exposing one or more populations of eukaryotic cells to a sample obtained from said subject, wherein each population of eukaryotic cells preferentially expresses an OR comprising an amino acid sequence selected from the group consisting of (1) an amino acid sequence selected from the group consisting of SEQ ID NOs: 1-40, (2) an orthologue or a paralogue of an OR comprising an amino acid sequence selected from the group consisting of SEQ ID NOs: 1-40, (3) amino acid sequence with greater than 85% identity to any one of SEQ ID NOs: 1-40, (4) amino acid sequence selected from the group consisting of SEQ ID NOs: 1 and 6-18, (5) an orthologue or a paralogue of an OR comprising an amino acid sequence selected from the group consisting of SEQ ID NOs: 1 and 6-18, (6) an amino acid sequence with greater than 85% identity to any one of SEQ ID NOs: 1 or 6-18, (7) an amino acid sequence of an OR described in any of Tables 1-5, and/or (8) an amino acid sequence with greater than 85% identity to an amino acid sequence of an OR described in any of Tables 1-5; (b) measuring in each of the one or more populations of eukaryotic cells the activation or lack of activation of the preferentially expressed OR by the one or more odorant molecules in the sample obtained from said subject; and (c) (1) diagnosing a disease or condition associated with the levels of one or more neurotransmitters or a change in the levels of one or more neurotransmitters in the CNS of a subject or (2) identifying an increased risk of developing a disease or condition associated with the levels of one or more neurotransmitters or a change in the levels of one or more neurotransmitters in the CNS when activation of one or more preferentially expressed ORs is detected in the one or more of the populations of eukaryotic cells. In some embodiments, the one or more populations of eukaryotic cells are one or more populations of OSNs, or cilia derived therefrom.

Provided herein are biosensors for detecting one or more odorants associated with a change in the levels of one or more neurotransmitters in the CNS of a subject as compared to the control levels for the one or more neurotransmitter. As used herein, a “control level” of a neurotransmitter in the CNS may refer to a level of that neurotransmitter in the CNS of one or more individuals who do not suffer from a disease associated with a change in the level of that neurotransmitter in the CNS. The control level may be measured on an individual-by-individual basis, or on an aggregate basis such as an average.

In some embodiments, a control level of a neurotransmitter in the CNS is the level is of that neurotransmitter in the CNS of one or more individuals who do not suffer from a disease associated with a change in the level of dopamine in the CNS. In some embodiments, a control level of a neurotransmitter in the CNS is the level is of that neurotransmitter in the CNS of one or more individuals who do not suffer from a disease associated with a change in the level of serotonin in the CNS. In some embodiments, a control level of a neurotransmitter in the CNS is the level is of that neurotransmitter in the CNS of one or more individuals who do not suffer from PD, depression, schizophrenia, dystonia, and/or restless leg syndrome.

In some embodiments, the one or more individuals are healthy individuals.

In some embodiments, the control level of a neurotransmitter in the CNS is the level of that neurotransmitter in the CNS in the same individual for whom a diagnosis is sought or whose condition is being monitored, but is obtained at a different time. A control level may refer to a level obtained from the same patient at an earlier time, e.g., weeks, months, or years earlier. A control level may refer to a level obtained from the same patient at a later time, e.g., weeks, months, or years later.

Likewise, a “control subject” refers to one or more individuals who do not suffer from a disease associated with a change in the level of that neurotransmitter in the CNS. In some embodiments, the control subject has a control level of a neurotransmitter in the CNS. In some embodiments, the control subject does not suffer from a disease associated with a change in the level of dopamine in the CNS. In some embodiments, the control subject does not suffer from a disease associated with a change in the level of serotonin in the CNS. In some embodiments, the control subject does not suffer from PD, depression, schizophrenia, dystonia, and/or restless leg syndrome. In one embodiment, the control subject is a healthy individual.

As used herein, a difference from a control level refers to a difference that is statistically significant, as determined by any statistical analysis method now or hereafter used by those in the art.

Once a subject is diagnosed with a disease or condition associated with the levels or a change in the levels of one or more neurotransmitters in the CNS of a subject, therapy can be initiated to treat, prevent, slow the onset of symptoms, or slow or halt the progression of the disease or condition associated with the levels or a change in the levels of one or more neurotransmitters in the CNS of a subject. The ability to detect diseases or conditions associated with a change in the levels of one or more neurotransmitters in the CNS of a subject early, may, for example, allow the early use of one or more therapies designed to slow or prevent onset of symptoms. Disease-modifying therapies are intended to prevent, slow or halt the progression of diseases and conditions associated with a change in the levels of one or more neurotransmitters in the CNS of a subject.

In some embodiments, the disease or condition associated with a change in the level of one or more neurotransmitters in the CNS of a subject is a neurodegenerative disease, including, e.g., PD. The ability to detect neurodegenerative diseases like PD early, before neurological symptoms appear, allows for the administration of therapeutic agents in order to prevent, or delay the onset or progression of, the neurodegeneration. Accordingly, in some embodiments, the subject is administered a neuroprotective agent. The term “neuroprotective” agent, as used herein, refers to agents intended to prevent neurodegeneration, including agents that slow down or stop the progression of neuronal degeneration. Neuroprotective agents include, but are not limited to, caffeine, nicotine, urate, UA, vitamin E, vitamin C, vitamin D, beta-carotene, riboflavin, coenzyme Q10, creatine, docosahexaenoic acid (DHA), uridine, uric acid, melatonin, glutathione (GSH), phytic acid (IP6), non-steroidal anti-inflammatory drugs (NSAID), isradipine, phenylbutyrate, exendin-4 (Ex-4), rasagiline, minocycline, EMD 1195686 (Safinamide), Zonisamide, GM1 ganglioside, and acetaminophen.

In some embodiments, a PD patient is administered levodopa-based preparations, designed to replace the dopamine in the depleted striatum. L-dihydroxyphenylalanine (levodopa or L-DOPA) is a dopamine precursor levodopa that is able to cross the BBB. After absorption and transit across the BBB, levodopa is converted into the neurotransmitter dopamine by DOPA decarboxylase. Administration of levodopa may cause side effects resulting from the conversion of levodopa to dopamine outside the CNS (peripheral conversion) by DOPA decarboxylase. Accordingly, in some embodiments, levodopa is administered in combination with peripheral inhibitors of DOPA decarboxylase, including, but not limited to, benserazide and carbidopa.

In some embodiments, the PD patient is administered a dopamine agonist which stimulates the activity of the dopamine system by binding to the dopaminergic receptors. In some embodiments, the dopamine agonist is administered during the initial therapy for PD.

In some embodiments, the dopamine agonist is an ergot-derived dopamine agonist including, but not limited to, bromocriptine (Parlodel), pergolide (Permax), cabergoline, or lisuride. In some embodiments, the dopamine agonist is a non-ergot-derived dopamine agonist including, but not limited to apomorphine (Apokyn), pramipexole (Mirapex), ropinirole (Requip), and rotigotine (NeuPro).

In some embodiments, the PD patient is administered an inhibitor of an enzyme involved in dopamine metabolism, wherein the inhibitor preserves the levels of endogenous dopamine.

In some embodiments, the inhibitor of inhibitor of an enzyme involved in dopamine metabolism is a MAO-B inhibitor, including, but not limited to, selegiline (Deprenyl, Eldepryl, Zelapar), rasagiline (Azilect), and safinamide (Xadago).

In some embodiments, the inhibitor of an enzyme involved in dopamine metabolism is an inhibitor of catechol-O-methyl transferase (COMT). Non-limiting examples of COMT inhibitors include entacapone (Comtan), tolcapone (Tasmar), and opicapone (Ongentys).

In some embodiments, the inhibitor of an enzyme involved in dopamine metabolism is used in combination with levodopa-based preparations and may allow for a reduction in the levodopa dose.

In some embodiments, the PD patient is administered an anticholinergic, which reduces the activity of the neurotransmitter acetylcholine by acting as an antagonist at cholinergic receptors. Non-limiting examples of anticholinergics include benztropine, orphenadrine, procyclidine, and trihexyphenidyl (Benzhexol).

In some embodiments, the PD patient is administered amantadine (Symmetrel) (which acts as a weak glutamate antagonist at the N-methyl-d-aspartate receptor (NMDAR)), Exenatide (Byetta), or Isradipine,

In some embodiments, the PD patient is administered gene therapy. Transgenes used in gene therapy for PD include, but are not limited to, genes encoding for DOPA decarboxylase, tyrosine hydroxylase (TH), and guanosine triphosphate cyclohydrolase-1 (GTPCH1). In some embodiments, the PD patient is administered ProSavin, a lentivirus vector comprising genes encoding DOPA decarboxylase, TH, and (GTPCH1). In some embodiments, the PD patient is administered a gene therapy targeting a gene linked to PD including, but not limited to, α-synuclein (AS) (SNCA), parkin (PARK2), UCH-L1 (PARKS), PINK1 (PARK6), DJ-1 (PARK7), leucine-rich repeat kinase-2 (LRRK2; PARKS), and ATP13A2 (PARKS). In some embodiments, the PD patient is administered gene therapy targeting a gene linked to an increased risk of developing PD including, but not limited to, GBA1, VPS35, EIF4G1, and PARK16.

In some embodiments, the PD patient is administered c-Abl tyrosine kinase inhibitor (including, but not limited to, nilotinib) or a glucagon-like peptide-1 receptor agonist (including, but not limited to, exenatide).

In some embodiments, the PD patient is administered a therapy that targets a protein or pathway known to play a role in PD, including antioxidants (glutathione, inosine) or Neurotrophic Factors (GDNF, CERE-120).

In some embodiments, the PD patient is administered a therapy that reduces α-synuclein production, inhibits α-synuclein aggregation, increases intracellular and extracellular degradation of α-synuclein aggregates, and/or reduces uptake of extracellular α-synuclein by neighboring cells. In some embodiments, the PD patient is administered affitope, NPT088, or NPT200-11.

In some embodiments, the PD patient is administered a cell-based therapy to replace nigrostriatal dopamine terminals lost by the disease process, with fetal or stem cell derived DA neurons placed directly into the caudate-putamen, and/or in substantia nigra. In some embodiments, induced pluripotent stem cells, embryonic stem cells, or universal donor cells are used for the cell-based therapy. In some embodiments, somatic cells are converted to dopamine neurons in vivo using virus technology.

In some embodiments, the PD patient receives deep brain stimulation (DBS).

### EXAMPLES

### Example 1. Identification of Odorant Receptors for the Detection of Odorants Associated with PD

To identify the ORs that are involved specifically in response to samples from patients with PD, a technique called “Deorphanization of Receptors based on Expression Alterations of mRNA levels” (DREAM) was used. This technique utilizes the generalized reduction in OR mRNA levels that occur after specific OR activation (von der Weid, B., Rossier, D., Lindup, M., Tuberosa, J., Widmer, A., Col, J. D., Kan, C., Carleton, A., and Rodriguez, I. (2015). Large-scale transcriptional profiling of chemosensory neurons identifies receptor-ligand pairs in vivo. Nat Neurosci 18, 1455-1463; see also US2017/0285009, both incorporated herein by reference).

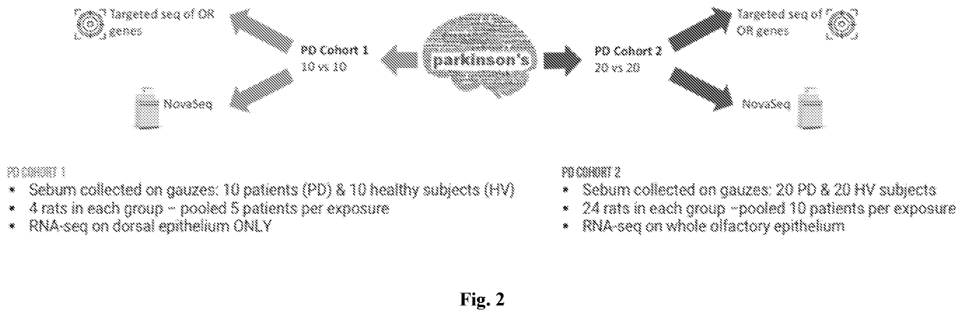

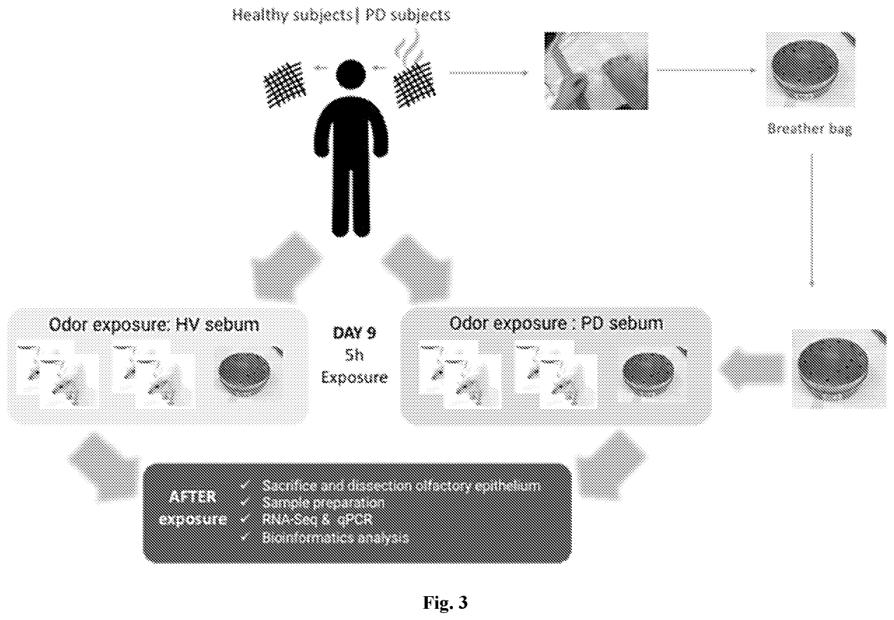

Samples were obtained from two independent cohorts of PD patients (PD1 and PD2) along with age matched healthy volunteers (HV) (n=10 in cohort 1 and n=20 in cohort 2 in each group), see FIG. 2. The samples consisted of sebum collected from every individual at the base of the back of the neck on gauzes. The gauzes in the first cohort (PD1) were inspected by Joy Milne, who can detect by smell PD, to validate the correct assignment of groups. The individuals were required to not wash or shower 48h prior collection of the sebum on gauzes.

Subsequently, the gauzes were cut up in pieces. Samples from PD patients and healthy volunteers respectively were pooled and placed in breather bags (similar to the bags used to train dogs to sniff-out explosives) (see FIG. 3). The breather bags were then placed in stash tins to be presented to the individually-housed animals. The DREAM assay was performed on wild type Lewis rats (n=4 per group with PD1 and n=24 per group with PD2) by exposing each group to the breather bags containing either the pooled PD gauzes or the pooled HV gauzes according to the rat group. Control rats were habituated to a blank odor for 48 hours. On day 3, the rats were split into the two groups of n=4 for PD1 and n=24 for PD2 and exposed to the samples. After 5 hours of odor exposure, rats were sacrificed and mRNA was extracted out of olfactory epithelial (OE) tissue. For the PD1 cohort only the dorsal portion of the OE tissue was extracted. For the PD2 cohort, a schedule of exposure was implemented, so that four rats in each groups were exposed to the respective odors (PD or HV), spreading 6 weeks for the entire set of 24 rats in each groups.

Differential gene expression (DGE) analysis was performed by sequencing the olfactory cDNA library for each animals and comparing the sequence results for the different groups. To generate the cDNA libraries, total RNA from each sample was quantified using a NanoDrop ND-1000 instrument. About 1 to 2 μg total RNA was used to prepare the sequencing library in the following steps: (1) Total RNA was enriched by oligo (dT) magnetic beads (rRNA removed); (2) RNA-seq library preparation using KAPA Stranded RNA-Seq Library Prep Kit (Illumina), which incorporates dUTP into the second cDNA strand and renders the RNA-seq library strand-specific. The completed libraries were qualified with Agilent 2100 Bioanalyzer and quantified by absolute quantification qPCR method.

Several rounds of different sequencing approaches were performed. A first round utilized a targeted capture approach amplifying the OR gene sets from rats. The second round of sequencing utilized a more shotgun sequencing approach using Illumina Novaseq to allow for a very deep sequencing averaging over 285 million reads per sample/animal with the PD2 cohort and over 100 million reads per sample/animal with the PD1 cohort.

Once fastq files were obtained from the different sequencing runs, sequence quality was examined using the FastQC software v11.9. The trimmed reads (trimmed 5′, 3′-adaptor bases using trimmomatic v0.39 and quality trimming) were aligned to reference genome Rnor6.0 (ensembl98) using STAR software v 2.7.3a along with the annotation gtf file for the reference genome. The transcript abundances for each sample was estimated with the “--quantMode GeneCounts” option within STAR, and the differential gene expression was analyzed with the DeSeq2 v1.26.0 package in R (version 3.6.3). An alternate sequencing analysis was performed in order to analyze the expression data under a secondary approach.

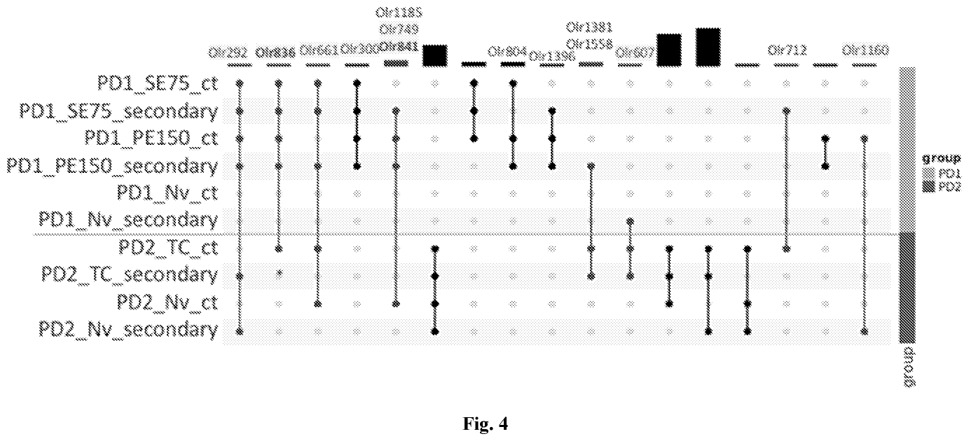

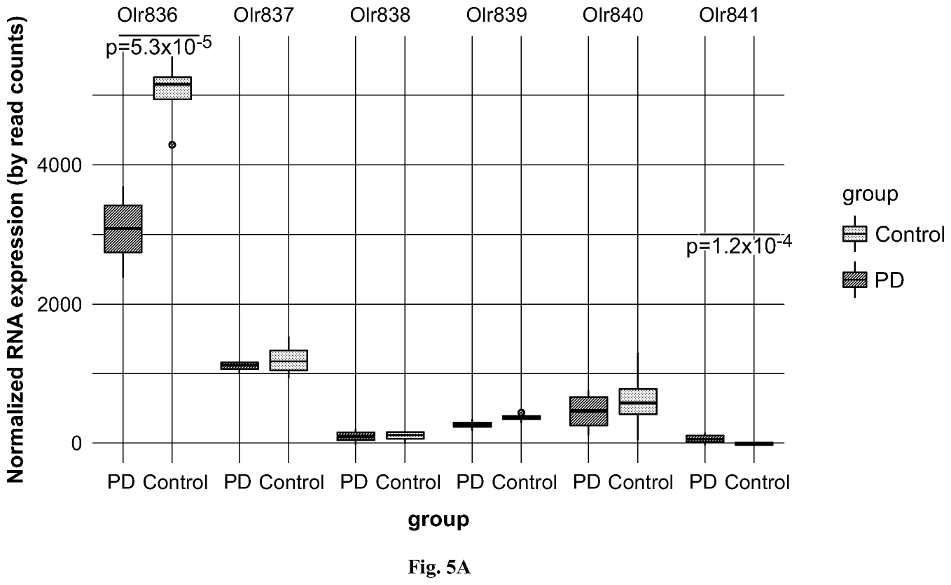

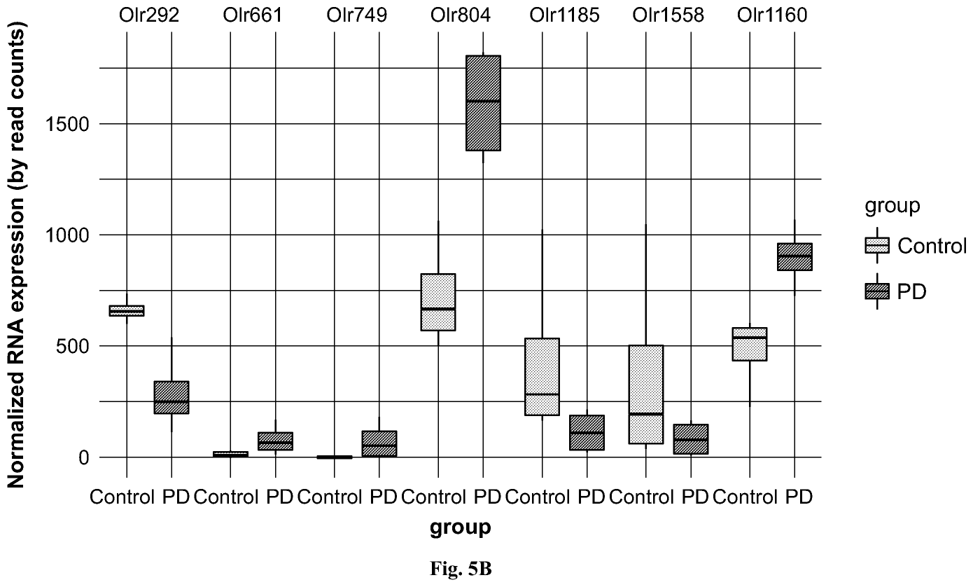

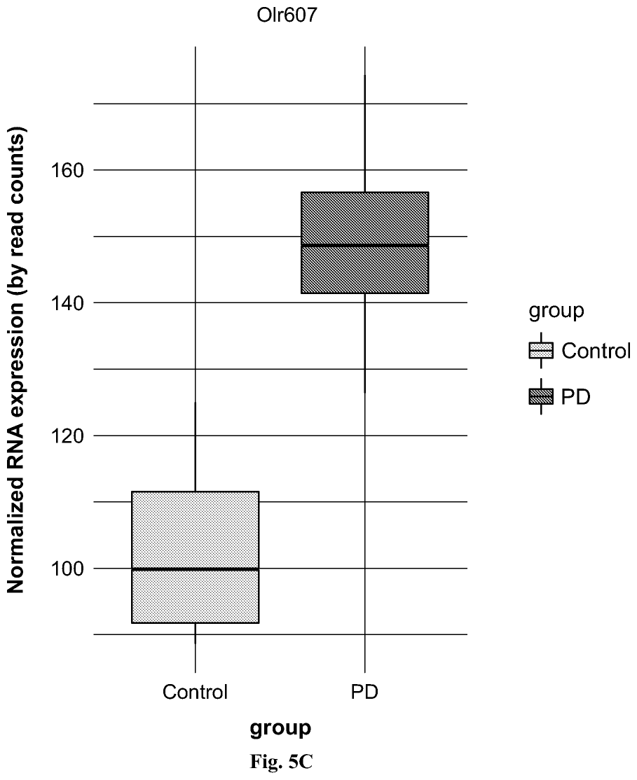

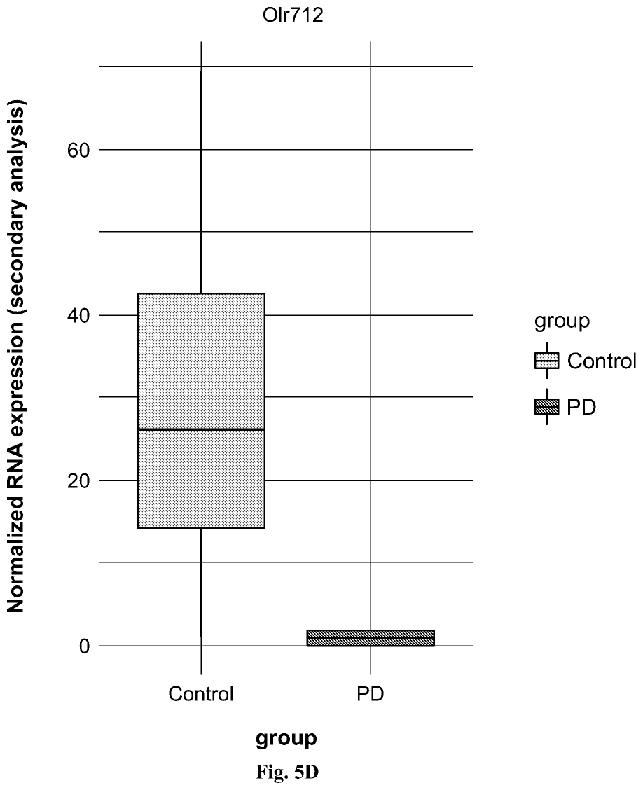

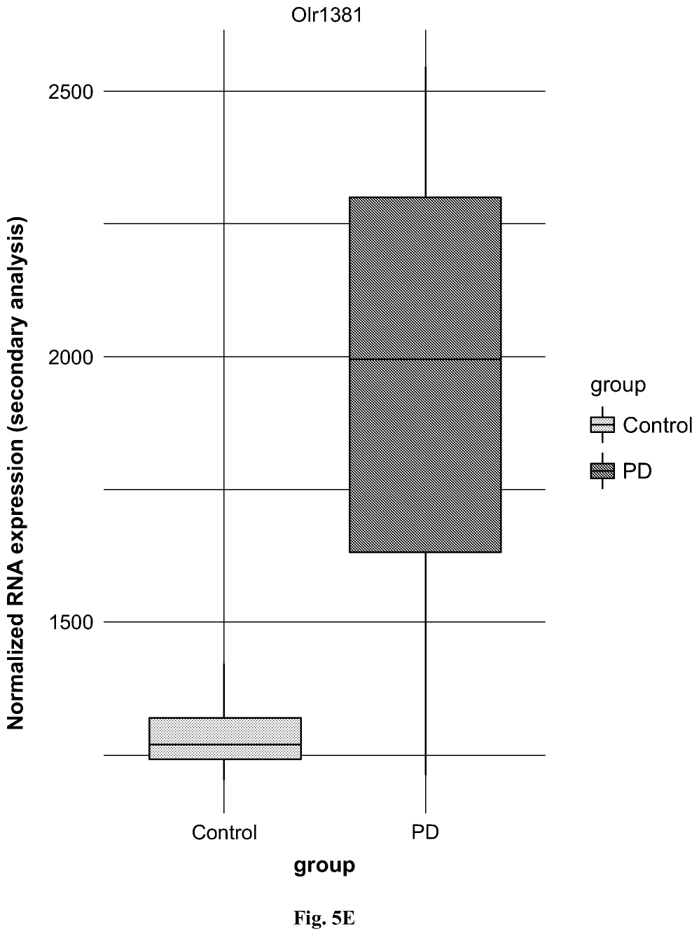

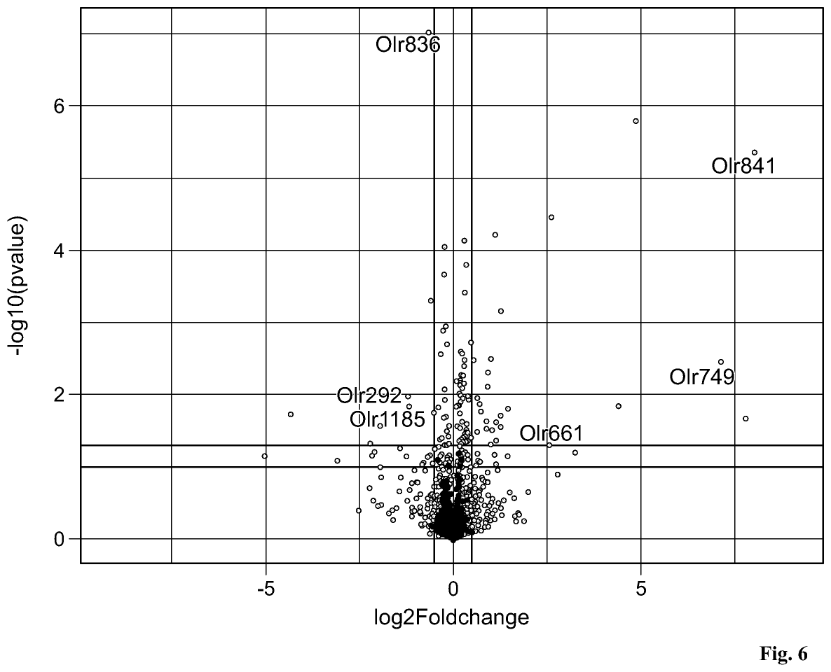

Eleven OR genes (Olr292, Olr836, Olr661, Olr841, Olr749, Olr1185, Olr1381, Olr1558, Olr607, Olr712, 041160) were identified as differentially expressed in both cohorts as compared to the control (highlighted with * in Table 1). See FIGS. 4-6. Three additional OR genes (Olr300, Olr1396 and Olr804) were identified in the PD1 cohort that were differentially expressed as compared to the control (highlighted with $ in Table 1). Olr836 and Olr841 belong to the subfamily of patch OR genes, a set of highly conserved genes believed to be activated by long chain aldehydes that can be found in sebum, but their specific ligands are unknown. Related patch genes that are conserved in human and mouse as well as the rest of the patch gene family in rats are provided in Tables 2 and 3.

In olfaction, it is assumed that every odor activates a subset of receptors, which is referred to as the odor combinatorial code. Here, a combinatorial code in rats was identified comprising 14 different rat ORs that are differentially activated by sebum from PD patients or from control sebum. The fact that the differentially activated ORs were identified in two different cohorts of PD patients indicates that this odor combinatorial code is characteristic for PD rather than patient-specific. The patients' disease unrelated, characteristic smell was controlled for by merging pieces of gauzes from separate individuals in order to minimize individual smells and amplify the PD-associated odorant signal to be captured. With samples from two independent cohorts, the calculated statistical power of this analysis was sufficient for the identification of ORs that are differentially activated by PD or control sebum samples.

TABLE 1
ORs identified in screen (Rattus norvegicus).

| | | SEQ ID | | | | |
|---|---|---|---|---|---|---|
| | | NO: | SEQ | NCBI | | |
| Gene | Organ- | (Amino | ID NO: | Gene | NCBI mRNA | NCBI Protein |
| Name | ism | Acid) | (cDNA) | ID | Accession No. | Accession No. |
| Olr836* | Rat | 1 | 41 | 298394 | NM_001000406.1 | NP_001000406.1 |
| Olr841* | Rat | 6 | 46 | 298049 | NM_001000405.1 | NP_001000405.1 |
| Olr300^{$} | Rat | 7 | 47 | 293599 | NM_001000237.1 | NP_001000237.1 |
| Olr1396^{$} | Rat | 8 | 48 | 405987 | NM_001001093.1 | NP_001001093.1 |
| Olr292* | Rat | 9 | 49 | 293594 | NM_001000232.1 | NP_001000232.1 |
| Olr804^{$} | Rat | 10 | 50 | 405142 | NM_001000852.1 | NP_001000852.1 |
| Olr661* | Rat | 11 | 51 | 405243 | NM_001000925.1 | NP_001000925.1 |
| Olr749* | Rat | 12 | 52 | 404804 | NM_001000615.1 | NP_001000615.1 |
| Olr1185* | Rat | 13 | 53 | 405337 | NM_001000982.1 | NP_001000982.1 |
| Olr1381* | Rat | 14 | 54 | 363549 | NM_001000523.1 | NP_001000523.1 |
| Olr1558* | Rat | 15 | 55 | 404978 | NM_001000725.1 | NP_001000725.1 |
| Olr607* | Rat | 16 | 56 | 295834 | NM_001000334.1 | NP_001000334.1 |
| Olr712* | Rat | 17 | 57 | 404816 | NM_001000624.1 | NP_001000624.1 |
| Olr1160* | Rat | 18 | 58 | 405341 | NM_001000985.1 | NP_001000985.1 |

*OR was identified as differentially expressed in both cohorts as compared to the control.
^{$}OR was identified in the PD1 cohort and was differentially expressed as compared to the control.
Olr836 and Olr841 are rat patch genes.

Once an OR has been identified, a person skilled in the art can identify homologous or orthologous proteins that fulfill the same function. A non-exhaustive list of orthologues and paralogues of the rat ORs in Table 1 can be found in Tables 3 and 4. A non-exhaustive list of orthologues and paralogues of patch genes can be found in Table 5. All sequences for the NCBI Gene IDs, as well as NCBI mRNA and protein accession numbers provided in Tables 1-4 are incorporated herein by reference.

TABLE 2
Rat patch genes, mouse orthologues and paralogues to rat patch genes
Olr836-Olr841 and human orthologues to all mouse patch genes.

| | | SEQ ID NO: | SEQ ID NO: | NCBI Gene | NCBI mRNA | NCBI Protein Accession |
|---|---|---|---|---|---|---|
| Gene Name | Organism | (Amino Acid) | (cDNA) | ID | Accession No. | No. |
| Olr837 | Rat | 2 | 42 | 405207 | NM_001000897.1 | NP_001000897.1 |
| Olr838 | Rat | 3 | 43 | 366362 | NM_001000581.1 | NP_001000581.1 |
| Olr839 | Rat | 4 | 44 | 298734 | NM_001000415.1 | NP_001000415.1 |
| Olr840 | Rat | 5 | 45 | 298733 | NM_001000414.1 | NP_001000414.1 |
| LOC500460 | Rat | 40 | 80 | 500460 | | |
| Olfr155 | Mouse | 19 | 59 | 29845 | NM_019473.1 | NP_062346.1 |
| Olfr156 | Mouse | 20 | 60 | 29846 | NM_019474.2 | NP_062347.2 |
| Olfr157 | Mouse | 21 | 61 | 100040268 | NM_019475.3 | NP_062348.1 |
| Olfr159 | Mouse | 22 | 62 | 29849 | NM_019476.1 | NP_062349.1 |
| Olfr275 | Mouse | 23 | 63 | 258857 | NM_146858.2 | NP_667069.2 |
| OR10X1 | Human | 24 | 64 | 128367 | NM_001004477.1 | NP_001004477.1 |
| OR13J1 | Human | 25 | 65 | 392309 | NM_001004487.1 | NP_001004487.1 |
| OR2S2 | Human | 26 | 66 | 56656 | NM_019897.2 | NP_063950.2 |
| OR13C3 | Human | 27 | 67 | 138803 | NM_001001961.1 | NP_001001961.1 |
| OR13C4 | Human | 28 | 68 | 138804 | NM_001001919.1 | NP_001001919.1 |
| OR13C8 | Human | 29 | 69 | 138802 | NM_001004483.1 | NP_001004483.1 |
| OR13C5 | Human | 30 | 70 | 138799 | NM_001004482.1 | NP_001004482.1 |
| OR13C2 | Human | 31 | 71 | 392376 | NM_001004481.1 | NP_001004481.1 |
| OR13C9 | Human | 32 | 72 | 286362 | NM_001001956.1 | NP_001001956.1 |
| OR13D1 | Human | 33 | 73 | 286365 | NM_001004484.1 | NP_001004484.1 |
| OR2K2 | Human | 34 | 74 | 26248 | NM_205859.2 | NP_995581.1 |
| OR9A4 | Human | 35 | 75 | 130075 | NM_001001656.2 | NP_001001656.1 |
| OR9A2 | Human | 36 | 76 | 135924 | NM_001001658.1 | NP_001001658.1 |
| OR6X1 | Human | 37 | 77 | 390260 | NM_001005188.1 | NP_001005188.1 |
| OR9K2 | Human | 38 | 78 | 441639 | NM_001005243.1 | NP_001005243.1 |
| OR13F1 | Human | 39 | 79 | 138805 | NM_001004485.1 | NP_001004485.1 |

Rat: Rattus norvegicus.
Mouse: Mus musculus.
Human: Homo sapiens.

TABLE 3
Rat patch genes (two of which were identified in the screen)
and their orthologues in mouse/human.

| | Orthologous ORs | | | | |
|---|---|---|---|---|---|
| | | | | SEQ ID | |
| | | | | NO: | SEQ ID |
| OR | | | | (Amino | NO: |
| (Rat) | Organism | Gene Name | Gene ID | Acid) | (cDNA) |
| Olr836 | Mouse | Olfr159-OR37E | 29849 | 22 | 63 |
| | Human | OR2S2/hOR37A/ | 56656 | 26 | 67 |
| | | OST715 | | | |
| Olr837 | Rat | | | | |
| Olr838 | Mouse | Olfr156 | 29846 | 20 | 61 |
| Olr839 | Mouse | Olfr157 | 100040268 | 21 | 62 |
| Olr840 | Mouse | Olfr155 | 29845 | 19 | 60 |
| Olr841 | Rat | LOC500460 | 500460 | 40 | 80 |
| | Mouse | Olfr275 | 258857 | 23 | 64 |
| | Human | OR13F1 | 138805 | 39 | 79 |

Rat: Rattus norvegicus.
Mouse: Mus musculus.
Human: Homo Sapiens.

TABLE 4
Orthologues for non-patch genes ORs identified in the screen.

| OR | Orthologous ORs | | |
|---|---|---|---|
| (rat) | Organism | Gene name | Gene ID |
| Olr292 | Rattus norvegicus (rat) | Olr298 | 293597 |
| | Mus musculus (mouse) | Olfr527 | 257939 |
| | Canis Lupus (dog) | OR12J | 491693 |
| Olr661 | Rattus norvegicus (rat) | LOC686900 | 686900 |
| | Mus musculus (mouse) | Olfr1208 | 258774 |
| Olr749 | Rattus norvegicus (rat) | Olr748 | 296001 |
| | Rattus norvegicus (rat) | LOC691368 | 691368 |
| | Mus musculus (mouse) | Olf1276 | 258390 |
| Olr1185 | Rattus norvegicus (rat) | LOC103693025 | 103693025 |
| | Rattus norvegicus (rat) | LOC690821 | 690821 |
| | Mus musculus (mouse) | Olfr872 | 258553 |
| | Mus musculus (mouse) | Olfr869 | 258550 |
| Olr1381 | Rattus norvegicus (rat) | Olr1382 | 287093 |
| | Rattus norvegicus (rat) | Olr1376 | 287084 |
| | Rattus norvegicus (rat) | Olr1380 | 405148 |
| | Rattus norvegicus (rat) | Olr1378 | 287086 |
| Olr1558 | Rattus norvegicus (rat) | Olr1559 | 288187 |
| | Mus musculus (mouse) | Olfr1560 | 405383 |
| | Rattus norvegicus (rat) | Olr180 | 258178 |
| | Mus musculus (mouse) | Olf181 | 259001 |
| Olr607 | Rattus norvegicus (rat) | LOC100912605 (5D14-like) | 100912605 |
| | Mus musculus (mouse) | Olfr1162 | 258105 |
| | Mus musculus (mouse) | Olfr1163 | 258638 |
| Olr712 | Rattus norvegicus (rat) | Olr716 | 404814 |
| | Rattus norvegicus (rat) | Olr714 | 405239 |
| | Mus musculus (mouse) | Olfr1249 | 257984 |
| | Mus musculus (mouse) | Olfr1253 | 258370 |
| | Mus musculus (mouse) | Olfr1251 | 259145 |
| Olr1160 | Rattus norvegicus (rat) | Olr1162 | 405167 |

TABLE 5
Orthologues of mouse patch genes. Instances in which the rat, dog, or human
orthologue to the mouse gene indicated has an additional paralogue
(with no corresponding mouse orthologue) are indicated with *.

| | Gene ID | | | |
|---|---|---|---|---|
| Mouse | in Rat | Rat | Dog | Human |
| Olfr417 | 405221 | Olr1598 | OR10X1 | OR10X1 |
| Olfr248 | 289260 | Olr1600 | | |
| Olfr1139-ps1 | | | | |
| Olfr70 | 298397 | Olr833 | OR13E | |
| Olfr71 | 298396 | Olr834 | OR13J1 | OR13J1 |
| Olfr159 | 298394 | Olr836 | OR13C7F | |
| Olfr29-ps1 | | | | |
| | 405207 | Olr837* | | |
| Olfr156 | 366362 | Olr838 | OR13C1 | |
| Olfr157 | 298734 | Olr839 | OR13C7G | |
| | | | OR13C7H* | |
| Olfr155 | 298733 | Olr840 | OR2S2 | OR2S2/hOR37A |
| | | | LOC609676 | |
| | | | (OR2S2-like) * | |
| | 103692344 | LOC103692344 | | |
| | | (OR13C7-like)* | | |
| | 103692345 | LOC103692345 | | |
| | | (OR13C7-like)* | | |
| Olfr275 | 298049 | Olr841 | OR13F1 | OR13F1 |
| | 500460 | LOC500460 | OR13F1B | |
| | | (similar to Olr841) * | | |
| Olfr273 | 298045 | Olr847 | OR13C3 | OR13C3/hOR37G |
| | 100912327 | LOC100912327 | | |
| | | (13C3-like) * | | |
| | 103690011 | LOC103690011 | | |
| | | (13C3-like) * | | |
| | 108348075 | LOC108348075 | | |
| | | (13C3-like) * | | |
| | 298044 | Olr848* | | OR13C4/hOR37F* |
| | 405146 | Olr844/LOC100912374 | | |
| | | (13C3-like) * | | |
| | 298046 | Olr845* | | |
| | 298042 | Olr850* | | |
| Olfr272 | 298040 | Olr851 | OR13C8 | OR13C8/hOR37H |
| | 298039 | Olr852* | OR13C2B* | OR13C2/hOR37K* |
| | | | OR13C2* | |
| | | | | OR13C5/hOR37I* |
| Olfr271-ps1 | | | | OR13C9/hOR37L |
| Olfr270 | 298038 | Olr853 | OR13D1 | OR13D1 |
| | | | OR13D1B* | |
| | | | OR13D3* | |
| Olfr267 | 366377 | Olr854 | OR2K2 | OR2K2 |
| Olfr461 | | | OR9A7 | |
| Olfr460 | 405144 | Olr799 | OR9A4C | OR9A4 |
| | | | LOC607362 | |
| | | | (OR9A4) * | |
| | | | LOC100855949 | |
| | | | (OR9A4) * | |
| | | | LOC491435 | |
| | | | (9A4-like) * | |
| Olfr459 | 502755 | RGD1562066 | | OR9A2 |
| | | (similar to ORZ6) | | |
| Olfr458 | 405372 | Olr801 | OR2R1 | |
| Olfr457 | 405332 | Olr802 | | |
| Olfr456 | 405143 | Olr803 | | |
| Olfr450 | 405330 | Olr810 | OR2Q1 | |
| | 405139 | Olr809* | OR2Q2* | |
| | 103690255 | LOC103690255 | | |
| | | (OR-like protein | | |
| | | OLF3) * | | |
| | 103690291 | LOC103690291 | | |
| | | (OR-like protein | | |
| | | OLF3) | | |
| Olfr986 | 300643 | Olr1341 | | OR6X1 |
| Olfr827 | 288822 | Olr1072 | OR9K7 | |
| | 288823 | Olr1071* | | |
| Olfr825 | 366816 | Olr1070 | OR9K2 | OR9K2 |
| Olfr826 | | | | |
| Olfr324 | 405054 | Olr1431 | | |
| Olfr49 | 290202 | Olr1646 | OR6E1 | |
| Olfr1481-ps1 | | | | |

### Example 2. Generation of a Transgenic Mouse Preferentially Expressing an OR

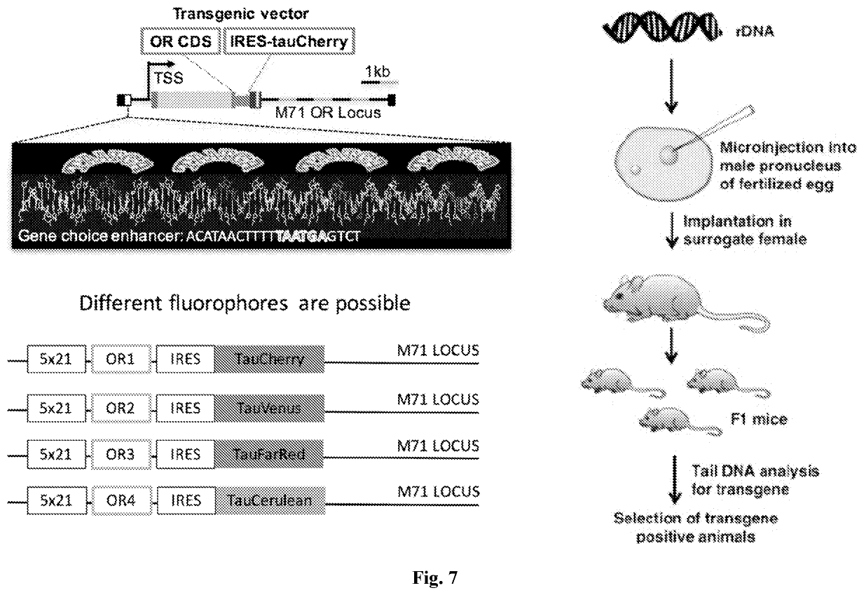

Genes encoding ORs were designed with Stu and AscI restriction sites flanking the two ends and synthesized as sequence-verified, double-stranded DNA fragments. These DNA fragments were digested with StuI and AscI, then ligated into the MouSensor vector (˜9 kB) (as described in D'Hulst C, Mina R B, Gershon Z, et al. MouSensor: A Versatile Genetic Platform to Create Super Sniffer Mice for Studying Human Odor Coding. Cell Rep. 2016; 16(4):1115-1125., incorporated herein by reference) digested with StuI and AscI. Ligated constructs were transfected into DH5alpha Escherichia coli cells, and positive clones were grown for plasmid purification. To create constructs expressing a different fluorescent reporter IRES-MP-Gcamp6f, the OR constructs were digested with PacI to isolate the OR fragment and ligated with PacI-digested reporter genes. The final constructs (˜10 kB) were digested with PmeI to linearize the DNA for pronuclear injection, in which the DNA randomly integrates into the mouse genome (FIG. 7). For this, purified DNA was microinjected into a fertilized oocyte, after which the zygote was reintroduced into a pseudopregnant female mouse (i.e., a female that was mated with a neutered male).

The resulting chimeric offspring are subsequently genotyped to verify incorporation of the transgene into the host genome. Molecular analysis of the founders (transgenic mice which have integrated the transgenic construct) was performed by utilizing an internal ribosomal entry site (IRES) in the OR expression vector that allows for bicistronic translation and simultaneous expression of the fluorophore (FIG. 7) with the OR enabling the visualization of the olfactory neuronal morphology in the brain. Using cryosections of OE and olfactory bulb (OB) tissue, a morphometric analysis of fluorescent transgenic glomeruli was performed and transgenic neuronal numbers were counted using confocal microscopy.

Because the transgenic ORs that were expressed in mouse OSNs are not necessarily of mouse origin, the transgenic OR-RNA levels were not compared with the endogenous mouse-OR RNA levels. To estimate the level of preferential expression of the transgenic ORs, a total glomerular volume (TGV) analysis was performed as a surrogate measurement for the calculation of the transgenic OSN numbers, because it is known that a positive correlation is present between the TGV and the number of OSNs expressing the corresponding OR (Bressel, O. C., M. Khan, and P. Mombaerts, J Comp Neurol, 2016. 524(1): 199-209, incorporated herein by reference).

### Example 3. Isolation of Cilia Derived from Olfactory Sensory Neurons Preferentially Expressing a PD-Discriminating OR

The olfactory epithelium from individual 6 to 8 week old, transgenic mice that preferentially express a PD-discriminating OR are dissected and washed briefly in cold buffer containing proteinase inhibitors. The buffer is replaced with solution containing calcium to “shock” the cilia off of the olfactory neurons (protocol adapted from Mayer et al. 2009; Kuhlmann et al. 2014, incorporated herein by reference). Tissue debris is removed by a brief centrifugation step. After two rounds (20 min shock and 10 min centrifugation) of the above shock procedure, the pooled supernatant is spun at high speed in an ultracentrifuge for 30 min at 4° C. The resulting cilia pellet is resuspended in buffer with 5% glycerol and proteinase inhibitors, aliquoted and flash-frozen in liquid nitrogen. Cilia aliquots are stored at −80° C.

### Example 4. Ex Vivo Bioassay Measuring OR Activation in Cilia

Cilia are obtained from a transgenic mouse preferentially expressing an OR described herein (generated using the methods described above). One μg of cilia bioextracts are incubated with 5 μM Forskolin or sample extract for 15 minutes at 37° C. in a total volume of 8 μL induction buffer (1×PBS, 100 μM Ro 20-1724[4-(3-butoxy-4-methoxybenzyl) imidazolidone], 500 μM IBMX (3-isobutyl01-methylxanthine)) in a covered white 96-well half-area plate. The cAMP-Glo™ assay (Promega) is performed on these samples according to manufacturer's suggestions adapted for 384-well plates. Forskolin (FSK) is used as a positive control to show viability of the cilia extracts. FSK binds directly with ACIII, which converts ATP into cAMP. Each sample (including controls) measurement is an average of technical triplicates.

### Example 5. Measuring Activation of PD-Discriminating ORs Upon Exposure of the ORs to Samples from PD Patients Ex Vivo

The assay employed to test activation of PD-discriminating ORs takes advantage of the fact that ORs are G-protein coupled receptors (GPCRs) that couple with adenylate cyclase III. Activated adenylate cyclase produces cyclic AMP (cAMP), which stimulates protein kinase A (PKA) activity, leading to a decrease in ATP levels. This decrease in ATP is measured using a luciferase reaction, using a commercially available assay, for example, the Promega cAMP-Glo™ Assay. In this assay, which can be adapted for a 384 well format, a lower level of ATP leads to decreased bioluminescence, indicating increased activity of the OR.

Sebum from a subject is collected on gauze, and extracted from the gauze using methanol. Extracted sebum samples can be stored at −80° C.

Freshly-thawed cilia (100-1,000 ng) isolated from either (i) mice that preferentially express an OR disclosed herein or (ii) wild type mice, are placed in triplicate wells and incubated with control (solvent alone) or sample for 15 minutes at 37° C. All subsequent steps are performed as per manufacturer's instructions for the Promega cAMP-Glo™ Assay. Analysis for cilia activation is performed by calculating the difference in the bioluminescent readout (DRLU) between PD-treated and control or untreated cilia for the cilia isolated from either (1) mice that preferentially express a PD-discriminating OR or (2) wild type mice.

For wild type cilia, neither a sample from a subject with PD nor the odor control causes activation of the ORs expressed in these cilia, and the ATP levels are about the same upon exposure of these cilia to either the odor control or the sample from a subject with PD. As such, the difference in DRLU observed for exposure to the odor control vs to the sample from a subject with PD is small.

For cilia isolated from mice that preferentially express a PD-discriminating OR, said OR is, for example, activated upon exposure to a sample from a subject with PD, leading to decreased ATP levels as compared to the same cilia exposed to the odor control. Therefore the difference in DRLU observed for exposure to the odor control vs to the sample from a subject with PD is significantly greater for these types of cilia.

Viability of the cilia is tested with Forskolin (5 μM). Forskolin (positive control) activates ACIII directly and increases the intracellular cAMP levels.

(Olr836 amino acid)
SEQ ID NO: 1
MDRSNETSLVSYFILLGLSAHPRLEKTFFVLILLMYLVILLGNGVLILVTILDSHLHTP
MYFFLGNLSFLDICYTTSSIPLVLDGFLTPRKTISFSGCAMQMFLSFAMGATECVLLG
MMAFDRYVAICNPLRYPVVMSKAAYVPMAAGSWAGGSITATVQTSLAMRLPFCGD
NVINHFTCEILAVLKLACADISINVISMAVGNAVFLGVPVLFIFVSYIFILSTILRIPSAE
GRKKAFSTCSAHLTVVIVFYGTILFMYGKPKSKDPLGADKQDLADKLISLFYGVLTP
MLNPIIYSLRNKDVRAAVRNLLHQKHFKW
(Olr837 amino acid)
SEQ ID NO: 2
MESSNQTSPVSRFILLGLSAHPTLEKTFFVLILLMYLVILLGNGVLILVTILDSHLHTPM
YFFLGNLSFLDICYTTSSVPLILDSFLTPRKTISFSGCAVQMFLSFAMGATECVLLSMM
AFDRYVAICNPLRYPVVMSKAVYVPMATGSWAAGIAASLVQTSLAMRLPFCGDNVI
NHFTCEILAVLKLACADISINIISMGVTNVIFLGVPVLFISFSYIFILSTILRIPSAEGRKK
AFSTCSAHLTVVIVFYGTILFMYGKPKSKDPLGADKQDLADKLISLFYGVLTPMLNPII
YSLRNKDVRAAVRNLVVLK
(Olr838 amino acid)
SEQ ID NO: 3
MEIANQSTVAEFVLLGLSDHPMLEKTFFVLILLMYLVILLGNGVLILVTILDSHLHTP
MYFFLGNLSFLDICYTTSSIPLVLDGFLTPRKTISFSGCAMQMFLSFAMGATECVLLG
MMAFDRYVAICNPLRYPVVMSKAAYVPMAVSSWVAGGANSMVQISLAVQLPFCGD
NIINHFTCEILAVLKLACADISINVISMGVANVIFLGVPVLFIFVSYIFILSTILRIPSAEGR
KKAFSTCSAHLTVVIVFYGTILFMYGKPKSKDPLGADKQDLADKLISLFYGVLTPML
NPIIYSLRNKDVKTAVRDLVGRKCLIQ
(Olr839 amino acid)
SEQ ID NO: 4
MEVSNQSTVTEFVLLGLSAHPTLEKTFFVLILLMYLVILLGNGVLILVTILDSHLHTPM
YFFLGNLSFLDICYTTSSVPLVLDGFLTPRKTISFSGCAVQMFLSFAMGATECVLLGM
MAFDRYVAICNPLRYPVVMSKAAYVPMAVSSWVAGGANSMVQISLAVQLPFCGDN
VINHFICEILAVLKLACADISINVISMGVANVIFLGVPVLFIFVSYIFILSTILRIPSAEGR
KKAFSTCSAHLTVVIIFYGTILFMYGKPKSKDPLGADKQDLADKLISLFYGLLTPMLN
PIIYSLRNKDVKTAVRDLASHRCLTQ
(Olr840 amino acid)
SEQ ID NO: 5
MEVSNQSTVTEFVLLGLSAHPTLEKTFFVLILLMYLVILLGNGVLILVTILDSHLHTPM
YFFLGNLSFLDICYTTSSVPLVLDGFLTPRKTISFSGCAVQMFLSFAMGATECVLLGM
MAFDRYVAICNPLRYPVVMSKAAYVPMAVSSWVAGGANSMVQISLAVQLPFCGDN
VINHFICEILAVLKLACADISINVISMGVANVIFLGVPVLFIFVSYIFILSTILRIPSAEGR
KKAFSTCSAHLTVVIIFYGTILFMYGKPKSKDPLGADKQDLADKLISLFYGLLTPMLN
PIIYSLRNKDVKTAVRDLASHRCLTQ
(Olr841 amino acid)
SEQ ID NO: 6
MVQGNWSSVTVFFFLGFSQYPRIEVTIFVLCLLMYLITLLGNTILISITILDSHLHTPMY
FFLSNLSFLDIWYTSSALTPMLANFVSGENTISFSGCALQMYFSLAMGSTECVLLSMM
AYDRYVAICNPLRYPIIMNRRVCVQIASSSWATGCLTALVETGPVIHLSLCGNNIINHF
TCEILALLKMACGDTAMVQLIMLVISILLLPLPMLLICVSYASILSNILRISSVDGRSKA
FSTCAAHLTVVVLFYGTALSMYLKPSSVNSQEIDKFMALIYAGLTPMLNPIIYSLRNK
EVKMAVKKLLIRNPFSTILTSVLK
(Olr300 amino acid)
SEQ ID NO: 7
MAPINQSVVTMFFLQNFVDDPWIQNVLFCFFFALFVAAIAGNGLIITVIHSSANLHTP
MYFFLVNLSLMDVICTVTVLPKVLQSLVAENAISYGGCLTQMFVFSWVLGSELLLFS
AMAYDRYLAICRPLHYGTLMSGRVCIALATFVWFTGALNSLVLTCLVLPLSFCGPNL
ITHFFCEIPSVLMLSCSPTFINDIMTVIADMFLTGLNFLLTMTSYGFIIASILRIRSAEGK
KRAFSTCSAHLVVVTLYYSTVLYTYVRPALGTSGLLDKVIAVLYTTVTPSLNPLIYTL
RNKEFKTSFKKLLFPN
(Olr1396 amino acid)
SEQ ID NO: 8
MDSFNATLEERFILVGFSDWPQLELILFVFISIVYSLTLFGNTAIIALSRMDLRLHTPMY
FFLSHLSFLDLCYTTSTVPQLLINLHGLDRIISYGRCVAQLFISLALGSTECVLLVVMA
FDRYAAVCRPLHYTTIMHPVLCQALAIASWVGGFLNSLIQTGLMMTIPLCGRRLNHF
FCEMPVFLKLACKDTGDTEAKMFVARAIILVFPATLILGSYGHIARAVLKVKSMAGR
RKAFGTCGSHLLVVSLFYGSAIYTYLQPKGSYSESDGKFVALFYTIVTPMLNPLIYTL
RNKDVKGALWKVLGRSTGQE
(Olr292 amino acid)
SEQ ID NO: 9
MMNGTLVTEFFILGFSEMPHLRVPLLLSFFCLYMAAISGNLLIMVTISASPALHTPMY
FFLVNLAMVDILCTSTILPKLLDSMMAGRSISYGGCMAQLFFFTWSLGAELLLFSAM
AYDRFVAICCPLHYSAWMGPRVCAFLAGLVWSISITNTSVHTGLMLRLPFCGSNEIE
HFFCEIPPLLKLSCAPTQLNEAMAFAADVFLAVGNFSVTILSYGFIVVSILRIRSAEGKR
RAFSTCSAHLIVVTMYYSTVIYTYIRPASSYSLNKDKVVSIIYTSVAPTLNPLIYTLRNK
DVKVALRRLLSCS
(Olr804 amino acid)
SEQ ID NO: 10
MDSPSNSTISRGFLLQGFFEFPHLRPLLFLVLLAVHLATLSGNLLILVAVVSVPSRPPM
LLFLCQLSAIELCYTLVVVPRSLADLSMPGHGRGSPISFLGCAVQMQMFVALGGAEC
FLLAAMAYDRYVAICHPLRYASIVTPGLCARLALACCLGGLAVSVGLTVAVFHLPFC
GSRLLVHFFCDITALLHLACTRSYIDELPLLGACLVLLLLPSMLILTSYGAIASALRRL
HGGGGRRKAASTCASHLAVTFLHYGCATFMYVRPKSSYSPRLDRTLALVYTNVTPL
LYPLIYSLRNREITSAIHRVLGLRGSGSM
(Olr661 amino acid)
SEQ ID NO: 11
MGYGNITEFILLGLFHDEDVKAICAVLFLLCYLAILCGNLIVLLTIKGSQLSEQPMYFF
LSYLSFMDVCFTSTVAPKFIIGLLVQCNTISYNACIAQMFYAHFFGATEIFILVVMAYD
RYVAICRPLYYMITMSRQVCYILVIGSVFGAFIHSLVHVLVIIRLPFCGTNEIDHYFCDI
FPLLKLACTETRLLVIVIITTTGVMSILTFVALVISYIIILSILRTRSSEGRRKALSTCGSHI
TVVFMFFLPLIFTYVPMGDSVGDDKVFALFYTMIAPLFNPLIYTLRNTDMKNAMRKV
WCQDKLFEGK
(Olr749 amino acid)
SEQ ID NO: 12
MNEINYTKVSEFVFLGLSKSKEIQHFFLAFSVVLYVAIVLGNTLVVFTLALDPHLHSP
MYFLLGNLSFIDLCFSTLTVPKMISDLSSGHNTISFHGCVFQIFVIHVLGASEMVLLVV
MAWDRYVAICKPLHYLTIMNPRICILLLSGAWIIAFLHSVIQLGFVVHLRFCGPNEIDS
FYCDLPRFIKLACMDNYRMEFMVAANSGIISIGTFFLLIISYIVILLTVRKHSSGDLSKA
LSTLSAHISVVVLFFGPCIFVYMWPFPTVPVDKFLAILDFMITPILNPAIYTLRNKDMK
VAMRKLSVQLLHFRKMS
(Olr1185 amino acid)
SEQ ID NO: 13
MSLSDNPELQPILFVLFLSFYMVTVLGNLLIILAVSSDFHLHTPMYFFLSNLSWSDICLI
STTVPKMIWDIRTQSRVISYAGCLTQMSMFIIFGCMDSMLLTVMAYDRFVAICHPLN
YNIIMNPNLCALLLLASVLASLVDSQVHNLIVLQFTYFNDMEISNFFCEPSQLLNLKCS
EMFTKNIVIRFIGVFFGLFSTSGIIFSYYKIVSSILRIPSTEGKYKAFSTCVSHLSVVCLFY
GTAIGVYIGSTASNSSENCKVASLMYTVVTPMLNPFIYSLRNRDIKTALWKLQKKAK
LHNAFHHFLILSS
(Olr1381 amino acid)
SEQ ID NO: 14
MSSTNHSSVSVFLLLGLSRQPQQQQLLFLLFLIMYLATVLGNLLIILSISTDSRLHTPM
YFFLSNLSFVDLCFSSTTVPKVLTNHILGSQEISFSGCLTQMYFLSVFADMDNFLLAV
MAYDRFVAICHPLHYTKKMTHQLCALLVVESWLAVNLNALLHTLLMARLSFCGDN
LIPHYFCDATPLLKLSCSDTHLNELMINTEGAVTMVTPFVCILISYIHITCAVLRVSSPR
GGWKAFSTCGSHLAVVCLFYGTIIAVYFNPSTAHSPEKDTVATVLYTVVTPMLNPFI
YSLRNRDLKWSLRKLVHRSTFSFQ
(Olr1558 amino acid)
SEQ ID NO: 15
MMKANHSLEFILIGFTDHPDLKTLLFLVFSAIYLVTMVGNLGLVALIYMEPRFHTPM
YIFLGNLALMDSCCSCAITPKMLENFFSVDRRISFCECMAQFYFLCLAETADCFLLAA
MAYDRYVAICNPLQYHTMMSKKLSLQMSMGIFIASNLHSLIHTGCLLRLNFCKSTKI
DHFFCDILPLYRLSCTNPFINELMIYIFSMPIQVFTITTILVSYFCILFTIFKMKSKDGRG
KAFSTCASHFFSVSIFYTCLLMYIGPSEDGSKDIPVAVFYTVIIPLLNPFIYSLRNKEVV
NAVKKVMKTYSIFKNASASIVH
(Olr607 amino acid)
SEQ ID NO: 16
MVPMERNVSVEIIFVLVGFTDYPELQIPLFLVFLFMYIITVVGNLGMIVLINIDPKFHTP
MYFFLSHLSFVDFCYPTIIMPKLLENLILADKTILYFSCMLQYFLSCVAVVAESYLLAV
MAYDRFVAICNPLLYTVAMSQRLCILLVTGSYIWSTLATLILLCYALQLKFSRFTVIN
HFFCEYTALIAVSSSDIHIPSLLLFCFATFNEVSTLLIILTSYVFIFVTVLKIKSASGRRKA
FSTCASHLTAITIFHGTILSLYCVPNPQNSRNAVKVASVFYAVVNPLLNPLIYSLRNKD
VKEVLQKVVSTSLKFPLH
(Olr712 amino acid)
SEQ ID NO: 17
MGQSNNVTEFVLLGFTQDPAGQKALFVMFSLMYIATMVGNLLIVGTVIVSPSLGSPM
YFFLASLSLMDAVYSTAISPKLIVDLLREKKTISFRACISQLFIEHLFGGVDIVILVAMA
YDRYVAICKPLHYLTIMNRRVCILFLVMAWAGGFAHALFQVLAVYNLPFCGPNIIDH
FGCDIYPLLLLACTDTYFIGLSVIGNNGAMCIVIFSLLLFSYGIILRSLQNHSQEGRRKA
LSTCSSHITVVFLFFVPCIFMYVRPVSSFPIDKSITVIYTVVTPMLNPLIYTLRNSEMKN
SMEKLLQKILSPNRLRLLCCLHY
(Olr1160 amino acid)
SEQ ID NO: 18
METYNLTGTLEFLLLDLSEDTELQFIFFVLFLLIYLLTVLGNVLIILAISSDSQLHSPMY
FFLYNLSLSDMGFSSTTVPKMLINMQTHNKSITYAGCLTQVFFFILFGGMDSLLLTAM
AYDRWVAICHPLHYQVILNPRLCRCLLILSFFISVLSSQVHCLMVSQLTFCTNMEIHHF
FCDVPELLKLSCSDSFISNIVRIILSIILGFLPMSGILYSYYKITSSIFKFPSLLGKYKVFST
CGSHLSVVCLFYGTGIGVYLSSTVSSSYGESMVASVLYIMVVPMINPFIYSLRNKDIK
KALQKIVRQIL
(Olfr155 amino acid)
SEQ ID NO: 19
MDRSNETAPLSGFILLGLSAHPKLEKTFFVLILMMYLVILLGNGVLILVSILDSHLHTP
MYFFLGNLSFLDICYTTSSVPLILDSFLTPRKTISFSGCAVQMFLSFAMGATECVLLSM
MAFDRYVAICNPLRYPVVMNKAAYVPMAASSWAGGITNSVVQTSLAMRLPFCGDN
VINHFTCEILAVLKLACADISINVISMVVANMIFLAVPVLFIFVSYVFILVTILRIPSAEG
RKKAFSTCSAHLTVVLVFYGTILFMYGKPKSKDPLGADKQDLADKLISLFYGVVTPM
LNPIIYSLRNKDVRAAVRNLVGQKHLTE
(Olfr156 amino acid)
SEQ ID NO: 20
MEGANQSTVAEFVLLGLSDHPKLEKTFFVLILLMYLVILLGNGVLILVSILDSHLHTP
MYFFLGNLSFLDICYTTSSIPLVLDGFLTPRKTISFSGCAVQMFLSFAMGATECVLLG
MMAFDRYVAICNPLRYPVVMNKSAYVPMAVSSWVAGGANSLVQISLAVQLPFCGD
NVINHFTCEILAVLKLACADISINVISMGVANVIFLGVPVLFIFVSYIFILSTILRIPSAEG
RKKAFSTCSAHLTVVLVFYGTILFMYGKPKSKDPLGADKQDVSDKLISLFYGVLTPM
LNPIIYSLRNKDVKAAVRNLVGQKCLIQ
(Olfr157 amino acid)
SEQ ID NO: 21
MDVSNQTTVTEFVLLGLSAHPKLEKTFFVLILSMYLVILLGNGVLILVSILDSHLHTP
MYFFLGNLSFLDICYTTSSVPLVLDGFLTPRKTISFSGCAVQMFLSFAMGATECVLLG
MMAFDRYVAICNPLRYPVVMNKAAYVPMAVSSWVAGGANSLVQISLAVQLPFCGD
NVINHFICEILAVLKLACADISINVISMGVANVIFLGVPVLFIFVSYIFILSTILRIPSAEG
RKKAFSTCSAHLTVVIIFYGTILFMYGKPKSKDPLGADKQDLADKLISLFYGLLTPML
NPIIYSLRNKDVKAAVRNLASHRCLTF
(Olfr159 amino acid)
SEQ ID NO: 22
MERSNKTTPVSSFILLGLSAHPKLEKTFFVLILLMYLVILLGNVVLILVSILDSHLHTPM
YFFLGNLSFLDICYTTSSVPLILDSFLTPRKTISFSGCAVQMFLSFAMGATECVLLGMM
AFDRYVAICNPLRYPVVMSKAAYVPMAAGSWVSGSITATVQISLAMTLPFCGDNVIN
HFTCEILAVLKLACADISINVISMAVANAMFLGVPVLFIFVSYIFILSTILRIPSAEGRKK
AFSTCSAHLTVVLVFYGTILFMYGKPKSKDPLGADKQDLADKLISLFYGVVTPMLNP
IIYSLRNKDVKAAVTNLVGQKHFKW
(Olfr275 amino acid)
SEQ ID NO: 23
MVQGNWTSVTVFVFLGFSHYPRIEVTVFVLCLLMYLITLLGNTILISITILDSHLHTPM
YFFLSNLSFLDIWYTSSALTPMLANFVSGKNTISFSGCASQMYFSLAMGSTECVLLSM
MAYDRYVAICNPLRYPIIMNRRVCVQIAGSSWVTGCLTALVETGPVIHLSLCGNSIIN
HFTCEILALLKLACGDTSMVQLIMLVISILLLPLPMLLICVSYASILSNILRISSMDGRSK
AFSTCAAHLTVVVLFYGTALSMYLKPSSVNSQEIDKFMALIYTGLTPMLNPIIYSLRN
KEVKMAVKKLLMKNPFSAILTSVLK
(OR10X1 amino acid)
SEQ ID NO: 24
MKINQTILKEFILVGFSVYPHVQTFLFVVFFCLYLLTLAGNLIIMGLTWVDRSLHTPM
YLFLSALSFSETCYTLTIVPKMLEDLLAKDRSISVTGCSLQMCFFLGLGGTNCIILTLM
GYDRFLAICNPLRYPLLMTNIVCGQLVASACTAGFFISLTETALIFRDSFCRPNLVKHF
FCHMLAVIRLSCIDSNHTEFIITLISVSGLLGTLLLIILTDVFIISTVLRIPSAEGKQKAFTT
CASHLTVVIIHFGFASIVYLKPEASGDDTLIAVPYTVITPFLSPIIFSLRNKDMKNAFRR
MMGNTVALKK
(OR13J1 amino acid)
SEQ ID NO: 25
MEPLNRTEVSEFFLKGFSGYPALEHLLFPLCSAMYLVTLLGNTAIMAVSVLDIHLHTP
VYFFLGNLSTLDICYTPTFVPLMLVHLLSSRKTISFAVCAIQMCLSLSTGSTECLLLAIT
AYDRYLAICQPLRYRVLMSHRLCVLLMGAAWVLCLLKSVTEMVISMRLPFCGHHV
VSHFTCKILAVLKLACGNTSVSEDFLLAGSILLLPVPLAFICLSYLLILATILRVPSAAR
CCKAFSTCLAHLAVVLLFYGTIIFMYLKPKSKEAHISDEVFTVLYAMVTTMLNPTIYS
LRNKEVKEAARKVWGRSRASR
(OR2S2 amino acid)
SEQ ID NO: 26
MEKANETSPVMGFVLLRLSAHPELEKTFFVLILLMYLVILLGNGVLILVTILDSRLHTP
MYFFLGNLSFLDICFTTSSVPLVLDSFLTPQETISFSACAVQMALSFAMAGTECLLLSM
MAFDRYVAICNPLRYSVIMSKAAYMPMAASSWAIGGAASVVHTSLAIQLPFCGDNVI
NHFTCEILAVLKLACADISINVISMEVTNVIFLGVPVLFISFSYVFIITTILRIPSAEGRKK
VFSTCSAHLTVVIVFYGTLFFMYGKPKSKDSMGADKEDLSDKLIPLFYGVVTPMLNPI
IYSLRNKDVKAAVRRLLRPKGFTQ
(OR13C3 amino acid)
SEQ ID NO: 27
MGEINQTLVSEFLLLGLSGYPKIEIVYFALILVMYLVILIGNGVLIIASIFDSHFHTPMYF
FLGNLSFLDICYTSSSVPSTLVSLISKKRNISFSGCAVQMFFGFAMGSTECLLLGMMAF
DRYVAICNPLRYPIILSKVAYVLMASVSWLSGGINSAVQTLLAMRLPFCGNNIINHFA
CEILAVLKLACADISLNIITMVISNMAFLVLPLMVIFFSYMFILYTILQMNSATGRRKA
FSTCSAHLTVVIIFYGTIFFMYAKPKSQDLIGEEKLQALDKLISLFYGVVTPMLNPILYS
LRNKDVKAAVKYLLNKKPIH
(OR13C4 amino acid)
SEQ ID NO: 28
MGEINQTLVSEFLLLGLSGYPKIEIVYFALILVMYLVILIGNGVLIIASIFDSHFHTPMYF
FLGNLSFLDICYTSSSVPSTLVSLISKKRNISFSGCAVQMFFGFAMGSTECLLLGMMAF
DRYVAICNPLRYPIILSKVAYVLMASVSWLSGGINSAVQTLLAMRLPFCGNNIINHFA
CEILAVLKLACADISLNIITMVISNMAFLVLPLMVIFFSYMFILYTILQMNSATGRRKA
FSTCSAHLTVVIIFYGTIFFMYAKPKSQDLIGEEKLQALDKLISLFYGVVTPMLNPILYS
LRNKDVKAAVKYLLNKKPIH
(OR13C8 amino acid)
SEQ ID NO: 29
MERTNDSTSTEFFLVGLSDHPKLQTVFFVLILWMYLMILLGNGVLISVIIFDSHLHTPM
YFFLCNLSFLDVCYTSSSVPLILASFLAVKKKVSFSGCMVQMFISFAMGATECMILGT
MALDRYVAICYPLRYPVIMSKGAYVAMAAGSWVTGLVDSVVQTAFAMQLPFCANN
VIKHFVCEILAILKLACADISINVISMTGSNLIVLVIPLLVISISYIFIVATILRIPSTEGKH
KAFSTCSAHLTVVIIFYGTIFFMYAKPESKASVDSGNEDIIEALISLFYGVMTPMLNPLI
YSLRNKDVKAAVKNILCRKNFSDGK
(OR13C5 amino acid)
SEQ ID NO: 30
MEWENHTILVEFFLKGLSGHPRLELLFFVLIFIMYVVILLGNGTLILISILDPHLHTPMY
FFLGNLSFLDICYTTTSIPSTLVSFLSERKTISLSGCAVQMFLSLAMGTTECVLLGVMA
FDRYVAICNPLRYPIIMSKDAYVPMAAGSWIIGAVNSAVQTVFVVQLPFCRNNIINHF
TCEILAVMKLACADISGNEFILLVTTTLFLLTPLLLIIVSYTLIILSIFKISSSEGRSKPSST
CSARLTVVITFCGTIFLMYMKPKSQETLNSDDLDATDKLIFIFYRVMTPMMNPLIYSL
RNKDVKEAVKHLLRRKNFNK
(OR13C2 amino acid)
SEQ ID NO: 31
MEWENHTILVEFFLKGLSGHPRLELLFFVLIFIMYVVILLGNGTLILISILDPHLHTPMY
FFLGNLSFLDICYTTTSIPSTLVSFLSERKTISLSGCAVQMFLGLAMGTTECVLLGMMA
FDRYVAICNPLRYPIIMSKDAYVPMAAGSWIIGAVNSAVQSVFVVQLPFCRNNIINHF
TCEILAVMKLACADISDNEFIMLVATTLFILTPLLLIIVSYTLIIVSIFKISSSEGRSKASST
CSAHLTVVIIFYGTILFMYMKPKSKETLNSDDLDATDKIISMFYGVMTPMMNPLIYSL
RNKDVKEAVKHLLNRRFFSK
(OR13C9 amino acid)
SEQ ID NO: 32
MEWENQTILVEFFLKGHSVHPRLELLFFVLIFIMYVVILLGNGTLILISILDPHLHTPMY
FFLGNLSFLDICYTTTSIPSTLVSFLSERKTISFSGCAVQMFLGLAMGTTECVLLGMMA
FDRYVAICNPLRYPIIMSKNAYVPMAVGSWFAGIVNSAVQTTFVVQLPFCRKNVINH
FSCEILAVMKLACADISGNEFLMLVATILFTLMPLLLIVISYSLIISSILKIHSSEGRSKAF
STCSAHLTVVIIFYGTILFMYMKPKSKETLNSDDLDATDKIISMFYGVMTPMMNPLIY
SLRNKDVKEAVKHLPNRRFFSK
(OR13D1 amino acid)
SEQ ID NO: 33
METRNYSAMTEFFLVGLSQYPELQLFLFLLCLIMYMIILLGNSLLIIITILDSRLHTPMY
FFLGNLSFLDICYTSSSIPPMLIIFMSERKSISFIGCALQMVVSLGLGSTECVLLAVMAY
DHYVAICNPLRYSIIMNGVLYVQMAAWSWIIGCLTSLLHTVLTMMLPFCGNNVIDHI
TCEILALLKLVCSDITINVLIMTVTNIVSLVILLLLIFISYVFILSSILRINCAEGRKKAFST
CSAHLIVVILFYGSALFMYMKPKSKNTNTSDEIIGLSYGVVSPMLNPIIYSLRNKEVKE
AVKKVLSRHLHLLKM
(OR2K2 amino acid)
SEQ ID NO: 34
MQGENFTIWSIFFLEGFSQYPGLEVVLFVFSLVMYLTTLLGNSTLILITILDSRLKTPM
YLFLGNLSFMDICYTSASVPTLLVNLLSSQKTIIFSGCAVQMYLSLAMGSTECVLLAV
MAYDRYVAICNPLRYSIIMNRCVCARMATVSWVTGCLTALLETSFALQIPLCGNLID
HFTCEILAVLKLACTSSLLMNTIMLVVSILLLPIPMLLVCISYIFILSTILRITSAEGRNKA
FSTCGAHLTVVILYYGAALSMYLKPSSSNAQKIDKIISLLYGVLTPMLNPIIYSLRNKE
VKDAMKKLLGKITLHQTHEHL
(OR9A4 amino acid)
SEQ ID NO: 35
MLMNYSSATEFYLLGFPGSEELHHILFAIFFFFYLVTLMGNTVIIMIVCVDKRLQSPM
YFFLGHLSALEILVTTIIVPVMLWGLLLPGMQTIYLSACVVQLFLYLAVGTTEFALLG
AMAVDRYVAVCNPLRYNIIMNRHTCNFVVLVSWVFGFLFQIWPVYVMFQLTYCKS
NVVNNFFCDRGQLLKLSCNNTLFTEFILFLMAVFVLFGSLIPTIVSNAYIISTILKIPSSS
GRRKSFSTCASHFTCVVIGYGSCLFLYVKPKQTQAADYNWVVSLMVSVVTPFLNPFI
FTLRNDKVIEALRDGVKRCCQLFRN
(OR9A2 amino acid)
SEQ ID NO: 36
MMDNHSSATEFHLLGFPGSQGLHHILFAIFFFFYLVTLMGNTVIIVIVCVDKRLQSPM
YFFLSHLSTLEILVTTIIVPMMLWGLLFLGCRQYLSLHVSLNFSCGTMEFALLGVMAV
DRYVAVCNPLRYNIIMNSSTCIWVVIVSWVFGFLSEIWPIYATFQFTFRKSNSLDHFY
CDRGQLLKLSCDNTLLTEFILFLMAVFILIGSLIPTIVSYTYIISTILKIPSASGRRKAFST
FASHFTCVVIGYGSCLFLYVKPKQTQGVEYNKIVSLLVSVLTPFLNPFIFTLRNDKVK
EALRDGMKRCCQLLKD
(OR6X1 amino acid)
SEQ ID NO: 37
MRNGTVITEFILLGFPVIQGLQTPLFIAIFLTYILTLAGNGLIIATVWAEPRLQIPMYFFL
CNLSFLEIWYTTTVIPKLLGTFVVARTVICMSCCLLQAFFHFFVGTTEFLILTIMSFDRY
LTICNPLHHPTIMTSKLCLQLALSSWVVGFTIVFCQTMLLIQLPFCGNNVISHFYCDVG
PSLKAACIDTSILELLGVIATILVIPGSLLFNMISYIYILSAILRIPSATGHQKTFSTCASH
LTVVSLLYGAVLFMYLRPTAHSSFKINKVVSVLNTILTPLLNPFIYTIRNKEVKGALRK
AMTCPKTGHAK
(OR9K2 amino acid)
SEQ ID NO: 38
MGDRGTSNHSEMTDFILAGFRVRPELHILLFLLFLFVYAMILLGNVGMMTIIMTDPRL
NTPMYFFLGNLSFIDLFYSSVIEPKAMINFWSENKSISFAGCVAQLFLFALLIVTEGFLL
AAMAYDRFIAICNPLLYSVQMSTRLCTQLVAGSYFCGCISSVIQTSMTFTLSFCASRA
VDHFYCDSRPLQRLSCSDLFIHRMISFSLSCIIILPTIIVIIVSYMYIVSTVLKIHSTEGHK
KAFSTCSSHLGVVSVLYGAVFFMYLTPDRFPELSKVASLCYSLVTPMLNPLIYSLRNK
DVQEALKKFLEKKNIIL
(OR13F1 amino acid)
SEQ ID NO: 39
MFPANWTSVKVFFFLGFSHYPKVQVIIFAVCLLMYLITLLGNIFLISITILDSHLHTPMY
LFLSNLSFLDIWYSSSALSPMLANFVSGRNTISFSGCATQVYLSLAMGSTECVLLPMM
AYDRYVAICNPLRYPIIMNRRTCVQIAAGSWMTGCLTAMVEMMSVLPLSLCGNSIIN
HFTCEILAILKLVCVDTSLVQLIMLVISVLLLPMPMLLICISYAFILASILRISSVEGRSK
AFSTCTAHLMVVVLFYGMALSMHLKPSAVDSQEIDKFMALVYAGQTPMLNPIIYSL
RNKEVKVALKKLLIRNHFNTAFISILK
(LOC500460, amino acid)
SEQ ID NO: 40
MVQGNWSSVTVFFFLGFSQYPRIEVTIFVLCLLMYLITLLGNTILISITILDSHLHTPMY
FFLSNLSFLDIWYTSSALTPMLANFVSGENTISFSGCAFQMYFSLAMGSTECVLLSMM
AYDRYVAICNPLRYPIIMNRRVCVQIAGSSWVTGCLTALVETGPVIHLSLCGNNIINH
FTCEILALLKMACGDTAMVQLIMLVISILLLPLPMLLICVSYASILSNILRISSVDGRSK
AFSTCAAHLTVVVLFYETALSMYLKPLSVNSQEIDKFMALIYVGLTPMLNPIIYSLRN
KEVKMAVKKLLIKNPFTAILTSVLK
(Olr836, cDNA)
SEQ ID NO: 41
atggacagatccaacgagacttcccttgtgtcctacttcattctcctcggcctctctgcccacccaaggctggagaaaaccttcttcgt
gctcatcctgctgatgtacctggtgatcctgctgggcaatggggtccttatcctggtgaccatcctcgactcccacttgcacacgccca
tgtacttcttcctggggaacctctccttcctggacatctgctacaccacttcctcaatccccttggtcctggatggttttctcacccccag
gaagaccatctccttctcaggctgtgccatgcagatgtttctgtccttcgccatgggagccacagagtgtgtgctcctgggcatgatgg
cgtttgatcgctatgtggccatctgcaacccccttaggtacccagtggtcatgagcaaggctgcctatgtccccatggccgccggctc
ctgggcagggggaagcatcactgccacagttcagacatctttagcaatgcggctgccattctgtggggacaacgtcatcaaccactt
cacctgtgagatcctggctgtcctgaaactggcctgtgccgacatctccatcaatgtcatcagcatggcggtaggtaatgcagtatttt
tgggggtcccagtcctgttcatctttgtctcctacatcttcatcctctccaccatcctgaggatcccctctgctgaggggaggaagaag
gccttctccacctgctctgcccacctcactgtggtgattgtcttctatgggaccatcctcttcatgtacgggaagcccaaatccaagga
cccactgggggcagacaagcaggaccttgcagacaagctcatctccctcttctatggggtgttgacccccatgttgaaccccatcatc
tacagcctgaggaacaaggacgtgagggccgctgtgaggaaccttctacatcagaaacacttcaagtggtga
(Olr837, cDNA)
SEQ ID NO: 42
Atggaaagtagcaaccagacctcccccgtgtcccgcttcattctcctgggcctctctgcccacccaaccctggagaaaaccttcttcgt
gctcatcctgctgatgtacctggtgatcctgctgggcaacggggtcctcatcctggtgaccatcctcgactcccacctgcacacgccca
tgtacttcttcctggggaacctctccttcctggacatctgctacaccacctcctcggtccccctcattctggacagcttcctgacccccagg
aagaccatctccttctcaggctgtgccgtgcagatgtttctgtctttcgccatgggagccacggagtgtgtgctcctgagcatgatggcgt
ttgatcgctatgtggccatctgcaatcccctcagatacccagtggtcatgagcaaggctgtctatgtccccatggccaccggctcctggg
cagctggaattgcagcctctctggttcaaacatcccttgcaatgcggctgcccttctgtggggacaacgtcatcaatcactttacctgtga
gatcctggctgtcctgaaactggcctgtgctgacatctccatcaatatcatcagcatgggggtgacaaatgtaatcttcctgggggtccc
agttctctttatttctttctcctacatattcatcctctccaccatcctgaggatcccctctgctgaggggaggaagaaggcAttctccacctg
ctctgcccacctcactgtggtgattgtcttctatgggaccatcctcttcatgtacgggaagcccaagtccaaggacccactgggggcag
acaagcaggaccttgcagacaagctcatctccctcttctatggggtgttgacccccatgttgaaccccatcatctacagcctgaggaac
aaggacgtgagggccgctgtgaggaacctggtggtccttaagtaa
(Olr838, cDNA)
SEQ ID NO: 43
Atggaaatagccaaccagtccacggtagctgagtttgtcttgctgggattgtcagaccacccaatgctggagaaaaccttcttcgtgct
catcctgctgatgtacctggtgatcctgctgggcaatggggtccttatcctggtgaccatcctcgactcccacctgcacacgcccatgta
cttcttcctggggaacctctccttcctggacatctgctacaccacttcctcaatccccttggtcctggatggttttctcactcccaggaaaac
catctccttctcaggctgtgccatgcagatgtttctgtctttcgccatgggagccacggagtgcgtgctcctgggcatgatggcgtttgat
cgctatgtggccatctgcaacccccttaggtaccccgtggtcatgagcaaggctgcctatgtgcccatggccgtcagctcctgggtgg
ctggcggtgccaactccatggtgcagatctcccttgctgtgcagttgcctttctgtggggacaatatcatcaaccacttcacctgtgagatt
ttggcagtcttaaagctagcctgtgctgacatctccatcaatgtcatcagcatgggggtggccaacgtgatcttcctgggggtcccagtt
ctgttcatctttgtctcctatatattcatcctctccaccatcctgaggatcccctctgctgaggggaggaagaaggcAttctccacctgctc
tgcccacctcaccgtggtgattgtcttctatgggaccatcctcttcatgtacgggaagcccaagtccaaggacccactgggggcagac
aagcaggaccttgcagacaagctcatctccctcttctatggggtgttgacccccatgttgaaccccatcatctacagtctgaggaacaag
gacgtgaagactgctgtaagagacctggtgggtcgaaaatgcctaattcagtga
(Olr839, cDNA)
SEQ ID NO: 44
Atggaagtgtccaaccagtccactgtgacagaatttgtcctattgggcctctctgcccacccaaccctggagaaaaccttcttcgtgctc
atcctgctgatgtacctggtgatcctgctgggcaatggggtcctcatcctggtgaccatccttgactctcacctgcacacgcccatgtact
tcttcctggggaacctctccttcctggacatctgctacaccacctcctcggttcccttggtcctggatggttttctcacccccaggaagacc
atctccttctcaggctgtgccgtgcagatgtttctgtctttcgccatgggagccacggagtgcgtgctcctgggcatgatggcgtttgatc
gctatgtggccatctgcaacccccttaggtaccccgtggtcatgagcaaggctgcctatgtgcccatggccgtcagctcctgggtggct
ggoggtgccaactccatggtgcagatctcccttgctgtgcagttgcctttctgtggggacaatgtcatcaatcatttcatttgtgagattttg
gcagtcttaaagctagcctgtgctgacatctccatcaatgtcatcagcatgggggtggccaacgtgatcttcctgggcgtcccagttctgt
tcatctttgtctcctacatcttcatactctccaccatcctgaggatcccctctgctgaggggaggaagaaggcAttctccacctgctctgc
ccacctcactgtggtaatcattttttatggtaccatcctcttcatgtacgggaagcccaagtccaaggacccactgggggcagacaagca
ggaccttgcagacaagctcatctccctcttctatggacttctgactcccatgttgaaccccatcatctacagcctgaggaacaaggatgtg
aagactgctgtaagagacctggcaagtcacagatgtctcacccagtaa
(Olr840, cDNA)
SEQ ID NO: 45
Atggacaggttcaatgagacctcccccgtatcccacttcattctcctgggcctctccgcccacccaaggctggagaaaaccttcttcgt
gctcatcctgctgatgtacctggtgatcctgctgggcaacggggtccttatcctggtgaccatcctcgactcccacctgcacacgcccat
gtacttcttcctggggaacctctccttcctggacatctgctacaccacctcctcggtccccctcattctggacagcttcctgacccccagg
aagaccatctccctctcaggctgtgccgtgcagatgtttctgtccttcgccatgggagccacggagtgtgtgcttctgagcatgatggcg
tttgatcgctatgtggccatctgcaacccccttaggtacccagtggtcatgagcaaggctgcctatgtgcccatggccgccggctcctg
ggcaggtggtatcaccaattctgtagttcagacatctttggcaatgaggctgccgttctgtggggacaatgtcatcaaccacttcacctgt
gagatcctggctgtcctgaaactggcctgtgctgacatctccatcaatatcatcagcatgggggtggccaacgtgatcttcctgggggtc
ccagttctgttcatctttgtctcctacatctttatcctctccaccatcctgaggatcccctctgctgaggggaggaagaaggcAttctccac
ctgctctgcccacctcaccgtggtgattgtcttctatgggaccatcctcttcatgtacgggaagcccaagtccaaggacccactggggg
cagacaagcaggaccttgcagacaagctcatctccctcttctatggggtggtgacccccatgttgaaccccatcatctacagcctgagg
aacaaggatgtgagggctgctgtgaggaacctggtggttcagaaacacctaactgagtga
(Olr841, cDNA)
SEQ ID NO: 46
atggtAcagggaaattggtcctctgtcacagtgtttttcttcctgggattttctcagtaccccagaattgaagtcaccatatttgtgctgtgt
ctgctgatgtacctgatcaccttgctgggaaatactattctgatctccatcactatccttgattctcacctgcacactcccatgtactttttc
cttagcaacctctcctttctggacatctggtatacctcttctgctctcactcctatgctggcaaactttgtttcaggggaaaacaccatctca
ttctcaggatgtgctttacagatgtacttctctcttgccatgggctccacggaatgtgtgctcctctctatgatggcctatgacaggtatgtg
gccatctgcaaccccctgagatatcccatcattatgaacaggagggtctgtgtacagattgcaagcagctcctgggctacaggctgcctcact
gccttggtggaaactggacctgtgattcatctgtctctctgtggtaataacatcatcaatcatttcacctgtgaaattctggctctcttaaaa
atggcttgtggagacactgccatggtgcagttaattatgttagtgatcagcatccttcttctcccattgccaatgttgctcatttgtgtctcc
tatgcatccatcctctccaacatcttgaggatcagctcagtggatggccgaagcaaagccttttcaacatgtgcagcccacttgactgtggtg
gttctgttctatgggacagctctctccatgtacctgaagccctcatctgtaaactcacaggaaatagataaatttatggcattgatatatgct
ggactaacaccaatgctaaatcctattatctacagtctacggaacaaagaagtcaaaatggctgtgaaaaaattgttgattagaaatccct
tcagtactatcttaacttctgtcctcaaataa
(Olr300, cDNA)
SEQ ID NO: 47
atggcAccaataaaccagtcagttgtgaccatgttcttcctgcaaaactttgttgatgatccctggatccagaatgttctcttttgctttttc
tttgccttgttcgtggcagccatagctggcaatggcctgattatcacagtcattcacagcagtgccaacctccacactcccatgtacttcttc
ctagtcaatctttccctcatggatgtgatttgcactgtgacagtgttgcccaaagtcctgcagagcctggtggcagagaacgccatttctta
tgggggatgtctcacacagatgtttgtcttctcctgggttctgggctctgagcttctgcttttctctgccatggcctacgaccgctaccttgc
aatctgccggccattgcactatggtaccctcatgagtggcagggtctgcatagcccttgcaacctttgtgtggttcactggagctctcaat
tccttggtgctcacttgtctggtgttgccactgtccttctgtggtcccaatctcatcacacacttcttctgtgagatcccttctgtgttgatg
ctgtcctgcagccccacctttatcaatgacatcatgactgtcattgcagacatgttccttacaggActgaacttcctattgactatgacatcc
tatggctttatcattgccagcatcctgcgcatccgctctgctgagggcaagaagcgtgccttctctacctgctctgcccacctggttgtggtc
accctttattattctactgttctatatacttatgtccggccagccctaggaacttctgggctcctggacaaagtcattgctgttctgtatacc
actgtgaccccatctctgaacccactcatctataccctgagaaacaaggaattcaaaacatcctttaaaaaactcttatttcccaattga
(Olr1396, cDNA)
SEQ ID NO: 48
Atggacagtttcaatgccactttagaagagagattcattttggtgggcttttcagattggcctcagctggaactcatcctttttgtttttatt
tcaattgtctactccctaactctctttggcaacaccgccatcatagctctctcccgaatggaccttcgattacacacccccatgtacttcttc
ctctcacacctctccttcctggacctctgctacaccaccagcaccgtgccccagctcctgatcaacctgcatggacttgacaggatcatca
gctatggaaggtgtgtggcccagctgtttatatctcttgctctgggatccactgagtgtgtgctcctggtggtgatggcttttgaccgctat
gctgctgtgtgtcgtccactgcactacacgaccatcatgcaccctgttctctgccaggcattggccattgcttcctgggtaggaggcttct
tgaactctctgattcaaacaggactcatgatgaccataccgctctgtggacgccgactaaatcatttcttctgtgagatgcctgtgttcctc
aagttagcctgcaaggacacaggagatacagaggccaagatgtttgtggccagagccataatcttggtcttccctgcaacattaattctt
ggctcctatggacacattgctagagcagtattgaaggtaaagtcaatggctggccgcagaaaggcttttgggacatgtgggtcccacct
cctggtggtttccctgttttatggctcagccatttacacatacttgcaacccaagggtagttattctgagagtgatggaaagtttgttgccct
tttttatactatagtgactcccatgctcaaccctttgatctataccctgaggaacaaggatgtgaagggcgctctgtggaaggtgctaggga
ggagcacaggccaagagtag
(Olr292, cDNA)
SEQ ID NO: 49
Atgatgaatgggacactggtcactgagttcttcatcctgggattctcagaaatgcctcaccttcgggtaccacttctcctcagcttcttctg
cctatacatggctgcaatctcaggaaacctgcttattatggtgacaatcagtgccagcccagccctgcatacccctatgtacttcttcctg
gtcaacttggccatggtggatatcctctgcacctccaccatcctaccaaagctactggacagcatgatggcagggaggagcatctctta
tgggggctgcatggcccagctcttcttcttcacatggtccctgggggcagagcttctgctcttctcagctatggcctatgaccgcttcgtg
gccatctgctgtcccctgcactacagtgcttggatgggccctagggtatgtgcattcctggctggccttgtctggtccatcagcataacta
acaccagcgtgcacacaggActgatgttgcgtctaccattctgtggctccaatgagatagagcacttcttctgtgagattcccccactgt
tgaaactctcctgtgctccaacacaattgaatgaggccatggcctttgctgcagatgtgttcctggctgtagggaacttctctgtgacaatc
ctctcctatggctttattgtggttagtatcctgaggatccgttcagctgaaggcaagcgacgtgctttttctacctgctctgcacacctcatt
gtggtcaccatgtactactccactgtcatctatacctacattcgccctgcatccagctactcactgaacaaggataaggtggtgtccatcatc
tacacctcggtggcacccaccttgaaccccctcatctacactctgagaaacaaggatgtcaaagttgcactccggagacttctatcctgc
tcctga
(Olr804, cDNA)
SEQ ID NO: 50
Atggacagccccagcaatagcaccatatcccgtggctttctccttcaaggcttttttgagtttccgcatctgaggccccttctcttcctggt
gctgctggctgtgcacctggccactttgagtggtaacctgctcatcctggtggctgtggtctcagtgcccagcaggccacccatgctgc
tcttcctttgtcagttgtcagccattgaactctgttacacgctagtggtggtgccccgatctctagccgacctgagcatgcctggccatgg
caggggcagccccatctccttcctgggctgtgctgtccagatgcagatgtttgtggcactgggtggggctgagtgcttcttgttggcagc
catggcctatgaccgctatgtggccatctgccacccactgcgctatgcatccattgtgaccccaggtctatgtgcacgactggccctgg
cctgctgccttggcggactagctgtatccgtgggactaacggtggcagtgttccacctccctttctgtggctcccgcctgctggtgcattt
cttctgcgacatcaccgccctgctgcacttggcctgcacacggagctacatagatgagctgcccttgctaggagcctgcctggtgctgc
tgttgctgccctcaatgctcatcctgacctcctatggggccattgcctctgccctgcgccgcctgcatggtggtggaggcagacgcaag
gctgcctcgacctgtgcttcacacctggctgtcaccttcttgcattacggctgtgccaccttcatgtacgtgcggcccaagtccagttact
ccccgcgactggatcgcacactagcgctggtttataccaacgtcacaccacttctatatcctctcatctacagcctgcgcaaccgggag
atcacttctgccattcacagagtcctggggctccgtggatcagggtctatgtga
(Olr661, cDNA)
SEQ ID NO: 51
Atgggatatgggaacatcacagaatttatcttactggggcttttccatgatgaggatgtcaaggcaatatgtgctgtgttattcttactatgc
tatcttgcaattctctgtggaaacttgattgttcttctcactatcaagggcagtcagcttagtgagcagccgatgtacttttttctcagctatct
gtctttcatggatgtctgcttcacttccacagtagccccaaaatttattataggactattggtacaatgtaacacaatctcctacaatgcctgc
atagcacaaatgttttatgcccacttctttggtgccactgagatatttatattggtggtcatggcctatgatcgctatgtagcaatttgcagac
ctctttactacatgatcacgatgagcagacaggtatgctacattcttgtgataggctcagtttttggtgcttttatacactcacttgtgcatgta
ttagttattatcagacttcctttttgtggcaccaatgaaatagaccactacttctgtgacatattccctctgctgaagctggcctgcactgaga
caagactccttgttattgtaatcattaccactacaggggtgatgtccattttgacttttgttgcattggtaatttcttacatcatcattttgtcc
atcttgaggaccaggtcatcggaaggccgtcgcaaagctctttctacctgtggctcacatatcaccgtagtgttcatgttcttcttgccactcat
ctttacctatgttccaatgggtgattctgttggggatgacaaggtgtttgctctgttttacaccatgattgcaccattgttcaaccctctcatct
acacactgaggaatacagacatgaagaatgccatgaggaaggtgtggtgccaagacaaactgtttgaaggaaagtga
(Olr749, cDNA)
SEQ ID NO: 52
Atgaatgaaataaattacaccaaggtttctgaatttgtgtttctgggactttcaaaatctaaagaaatacaacatttcttccttgccttctctgt
ggtattatatgtagccatcgtactgggcaatacccttgttgtgtttacactagcattggacccacatttacattcccctatgtattttcttttag
gaaacctctcatttattgatttatgtttttccaccttaactgtacccaagatgatttctgacttgtcgtctgggcacaataccatctcattccat
ggttgtgtcttccagatatttgtcattcatgtccttggggcttctgagatggtattgctggtagtcatggcctgggacagatatgtagccatatg
taaacccctccactatttaaccatcatgaacccacggatttgcattttgcttctatctggggcttggattattgcatttttgcattcagtgatcc
aacttggttttgttgtccatttgaggttttgtggtccgaatgagatagatagtttttactgtgatcttcctagattcatcaagctggcctgcatg
gacaactacagaatggagttcatggtggctgccaacagtggcatcatttctattggcaccttcttcttattgattatctcctacattgttatcct
gctcactgtgaggaaacattcatcaggagatttgtccaaggccctctcgacactttctgctcacatctctgtggtagttttgttctttggaccat
gcatctttgtatacatgtggccatttcctacagtgccagtggataagttccttgccattctggacttcatgattacacccatcctgaaccctg
ccatttacacactgaggaacaaagacatgaaggtggcaatgaggaaactaagtgttcagctcctgcattttaggaaaatgtcctga
(Olr1185, cDNA)
SEQ ID NO: 53
atgagcctctcagacaatccagaactgcagcctatcctttttgtactgtttttatcattctacatggtcacagtgcttggaaatctgctcattat
tctggctgtgagctctgacttccatctccatacccccatgtatttctttctctccaacctttcctggtctgacatttgtttgatctccactacag
ttccaaagatgatttgggatattagaactcaaagcagagtcatctcctatgcgggctgcctaacacagatgtccatgttcataatttttggatg
tatggatagcatgcttctgactgtaatggcctatgacaggtttgtggccatttgtcaccccctgaattacaatatcattatgaatcctaacctc
tgtgccctcttacttttggcatctgtattggccagtcttgtggattcgcaggtgcacaatttgattgtgctacaatttacatacttcaatgacat
ggaaatctctaactttttttgtgagccttcacaactgcttaatctcaagtgttctgaaatgttcaccaaaaacatagttatacgattcattggtg
tcttttttggtttattttcaacctcaggaattattttctcatactataaaattgtgtcttccatcctgagaattccatcaacagaaggaaagtat
aaagccttttctacctgtgtgtctcacctgtcagttgtgtgcttattttatggaacagccattggagtatacattggttcaactgcatcaaactc
ttctgaaaactgtaaagttgcttcactgatgtacacagtggtcacacctatgcttaatcccttcatctacagcctgaggaacagggacattaa
aactgccctatggaaactacagaaaaaagcaaaattgcacaatgccttccatcattttttaattttgagttcttaa
(Olr1381, cDNA)
SEQ ID NO: 54
Atgagcagcaccaaccattccagtgtctcagtgttcctcctcctgggactctccaggcagccccagcagcagcagctcctcttcctgct
cttcctcatcatgtacctggccactgtcctgggaaacctgctcatcatcctgtccatcagcacagactcccgcctgcacacccccatgta
cttcttcctcagcaacctgtcctttgtggatctctgcttctcctccaccactgtccctaaagtactgaccaaccatatacttgggagtcagga
aatttccttctctgggtgtctcacacagatgtattttctctctgtgtttgctgatatggacaattttctgctggctgtaatggcctatgaccgat
ttgtggccatatgccaccctttacactacacaaaaaagatgacccatcagctgtgtgcccttcttgttgttgagtcatggctggctgtcaatct
gaatgctcttttacatacactgctaatggctcgactttctttctgtggggacaacctcataccccactatttctgtgatgcaactcctctcctg
aaactctcctgctcagacacacatctcaatgagctaatgattaatacagagggagctgtgaccatggtcaccccatttgtctgcatcctga
tctcctacatccacatcacctgtgctgtcctgagagtctcatcccccaggggaggatggaaagccttctccacctgtggctcccacctgg
ctgtggtctgcctcttctatggcaccatcatcgctgtgtatttcaatccctcaactgcccattcacctgagaaggacacagtagccactgtg
ttgtacacagtggtgactcccatgctgaacccctttatctatagcctgaggaacagggacctgaaatggtcactacgaaaacttgtccat
agaagtaccttttcttttcagtga
(Olr1558, cDNA)
SEQ ID NO: 55
Atgatgaaggcaaatcactctttagagttcattctcataggattcacagatcacccagacctaaagacacttctgttcctggtgttctctgc
catatatctggtcaccatggtggggaatcttgggctggtggccttgatctacatggagcctcgtttccacacacccatgtacatctttctgg
gaaacctggccctgatggactcctgctgctcctgtgccatcactcccaagatgctagagaacttcttttctgtggacagaaggatctcttt
ctgtgaatgcatggcacagttctatttcctctgtcttgctgaaactgcagactgctttcttctggcagccatggcctatgaccgctatgtggc
catatgcaacccactgcagtaccacaccatgatgtccaagaagctctcccttcagatgagcatgggcatattcatagccagtaacctgc
attccttgattcatacaggctgtctgttaagattaaatttctgtaaatctaccaaaattgatcacttcttctgtgatattcttcccctgtatcg
cctctcctgtacaaacccttttattaatgaactaatgatatatattttttcaatgccaattcaagtctttaccattaccactatcttggtctctt
acttctgcattcttttcactattttcaagatgaaatccaaggatgggagaggaaaagcattttctacttgtgcatcccattttttttctgtgtca
atattctacacctgtcttctcatgtatattggaccatctgaagatggtagtaaagacataccagtagctgtgttttatacagtaataattccttt
gttaaacccttttatttacagcctgagaaataaggaggtagtaaatgctgttaagaaagttatgaaaacttacagtatttttaaaaatgcttctg
cttccatagtacattaa
(Olr607, cDNA)
SEQ ID NO: 56
atggtAccaatggaaagaaatgtgagtgtggagattatctttgtcctggtgggcttcacagactaccctgagcttcagatccccctcttcc
tagtctttctcttcatgtacatcatcactgtagtggggaaccttgggatgatagtgctcatcaatatcgaccccaagttccacactcccatgt
actttttccttagccacctttcctttgttgatttctgttatccaacaattatcatgcccaagctgcttgagaacttgatcctggctgataaaact
atactttatttcagctgcatgctgcagtacttcctgtcctgtgtggctgtggtggctgagtcctacttgttagcagttatggcctatgaccgctt
tgtggccatctgtaatcccttgctgtatactgtggccatgtcacagagactgtgcatcctgcttgtgactggatcatatatctggagcacatt
ggccaccttgattctcctctgctatgctctgcagctaaagttctcaagatttactgtgatcaaccacttcttctgtgaatatactgccctcattg
ctgtctctagttctgatatacacatccccagcctactactcttttgttttgcaacctttaacgaagtgagtaccctactgatcattctcacttct
tacgtgttcatttttgtgactgtgttaaaaatcaaatctgctagtggacgtcgcaaagccttctctacctgtgcctcccacctgactgccatca
ccatttttcatggaaccatactttctctgtactgtgtacctaacccacaaaattctaggaatgcagtcaaagtggcctctgtcttttacgcagt
tgtcaaccccttgcttaaccctcttatctacagtctgagaaacaaggatgtgaaagaggttttacagaaagtagtgagcacttcattgaaat
tcccacttcactga
(Olr712, cDNA)
SEQ ID NO: 57
atgggAcagagcaacaatgtcacagaatttgtcctgctgggcttcactcaggatcctgctgggcaaaaagctttatttgttatgttttcact
gatgtacattgcaacaatggtgggcaacctgctcattgtagggacagtgattgtcagcccctccttaggctccccgatgtacttcttccttg
cctctctgtcactcatggatgctgtttattctactgccatctcacccaagttgattgtagacttgctccgtgagaaaaagaccatctccttca
gagcttgcataagccagctctttatagagcatttatttggcggggttgacattgttattctggtggcaatggcctatgatcgctatgtggcca
tctgtaagccactacattatttgaccatcatgaatcgacgggtatgtattctcttcttggtgatggcctgggcaggaggttttgcacatgcttt
gtttcaagttcttgctgtatataatcttcctttctgtggtcccaatatcattgaccactttgggtgtgacatatacccgctcttgctacttgca
tgtactgacacctacttcattggactttctgtcattggcaataatggggccatgtgtatagtcatctttagtctccttctattttcctatggaa
taattcttagatctctccagaatcacagtcaggaagggaggcgcaaagctctgtccacctgcagctctcatatcactgtggtttttctcttttt
tgttccctgtattttcatgtatgttagacctgtttctagttttcctattgacaaatccatcactgtaatttatactgttgtcacccccatgttg
aatcctttaatatatactttgagaaattcagagatgaaaaattctatggaaaagctcctgcagaaaatattaagtccaaatagactaaggcttc
tttgctgcttacattattaa
(Olr1160, cDNA)
SEQ ID NO: 58
atggaAacatataatttaacaggaaccttggaatttcttctgcttgatctgtcagaggacactgaactgcagttcatcttctttgtattgttcct
gctcatatacctgctcactgtacttggaaatgtgctcatcatcttggccattagctctgattctcagctccatagtcctatgtactttttcctct
acaacctttcattgtctgacatgggcttcagcagcaccacagtccccaaaatgctgataaatatgcagactcacaacaaatccataacttat
gcaggctgcttaactcaggtattcttcttcattctttttgggggtatggacagcctattactgactgcaatggcctatgaccgatgggtagc
catttgtcaccctctacactaccaagtcattctcaaccctcgtctgtgtagatgtttgctcatactttcatttttcattagtgttttgagttcac
aggtacactgcttgatggtgtcacaattaacattttgcactaatatggaaatccatcatttcttctgtgatgttccagaacttctaaaactttcc
tgttctgactcctttatcagtaacatagttaggattattttaagcatcattcttggtttcctacccatgtcaggaatactttattcctactataa
aataacttcctccatttttaaatttccatcactgttagggaaatataaagtcttctctacttgtggatctcacctatcagttgtttgcttatttt
atggaacaggtataggtgtgtaccttagttcaacagtttctagttcttacggggaaagcatggtggcttcggtactatatatcatggtggttccc
atgataaaccccttcatctacagcctgaggaacaaggacatcaaaaaagcccttcagaaaattgtcagacaaatattgtag
(Olfr155, cDNA)
SEQ ID NO: 59
Atggacagatccaatgagaccgcccccctgtccggcttcattctcctgggcctctctgcccacccaaagctggagaaaaccttcttcgt
gctcatcctgatgatgtacctggtgatcctgctgggcaacggcgtcctcatcctggtgagcatcctcgactcccacctgcacacgccca
tgtacttcttcctggggaacctctccttcctggacatctgctacactacctcctctgtccccctcattctggacagctttctgactcccagga
agaccatctccttctcgggctgtgccgtgcagatgtttctctccttcgccatgggagccacggagtgtgtgctcctgagtatgatggcgtt
tgatcgttatgtggccatctgcaacccccttagatatcctgtggtcatgaacaaggctgcctatgtgcccatggctgccagttcctgggca
ggtggtatcactaattctgtagtgcagacatctttggcaatgcggctgcccttctgtggggacaatgtcatcaatcacttcacctgtgagat
cctggcagtcctgaaactggcctgtgctgacatctccatcaatgtcatcagcatggttgtggccaacatgatcttcttggcagtcccagtc
ctcttcatctttgtctcctatgtcttcatccttgtgacaatcctgaggatcccctctgctgaggggaggaagaaggcAttctccacctgctc
tgcccacctcaccgtggtacttgtcttctatggaaccatcctcttcatgtacgggaagcccaagtccaaggacccactgggggcagaca
agcaggaccttgcagacaagctcatctccctcttctatggagtggtgacccccatgctaaaccccatcatctacagcttgagaaacaag
gacgtgagggctgctgtgaggaacctggtgggccagaaacacctaactgagtga
(Olfr156, cDNA)
SEQ ID NO: 60
Atggaaggagccaatcagtctacggtagctgaatttgtcttgctggggttgtcagatcacccaaagctggagaaaaccttcttcgtgctc
atcctgctgatgtacctggtgatcctgctgggcaacggcgtcctcatcctggtgagcatcctcgactcccacctgcacacgcccatgta
cttcttcctggggaacctctccttcctggacatctgctatactacctcctcgattcccttagtcctggatggttttctcacccccaggaagac
catctccttctcgggctgtgccgtgcagatgtttctctcctttgccatgggagccacggagtgtgtgctcctgggcatgatggcgtttgatc
gttatgtggccatctgcaaccctcttagataccctgtggtcatgaacaagtctgcctatgtgcccatggccgtcagctcctgggtggctg
gcggtgctaactccttggtgcagatctcccttgcggtgcagttgcctttctgtggggacaatgtcatcaatcacttcacctgtgagatccta
gcagtcttaaagctagcctgtgctgacatctccatcaatgtgatcagcatgggggtggccaatgtgattttcctgggggttccagttctgtt
catctttgtctcctacatcttcatactctccaccatcctgaggatcccctctgccgaggggaggaagaaggcAttctccacctgttctgcc
cacctcaccgtggtacttgtcttctatgggaccatcctgttcatgtacgggaagcccaagtccaaggacccattgggggcagacaaaca
ggatgtttcagacaagctcatctccttattttatggggtgctgacccccatgctgaaccccatcatctacagcctgaggaacaaggatgtg
aaggccgctgtgaggaacctggtaggtcagaaatgcctgattcagtga
(Olfr157, cDNA)
SEQ ID NO: 61
Atggatgtatccaaccagacgactgtaacagaatttgtcctgttgggcctctccgcccaccccaaactggagaaaaccttcttcgtgctc
atcttgtcaatgtacctggtgatcctgctgggcaacggggtcctcatcctggtgagcatcctcgactcccacctgcacacgcccatgtac
ttcttcctggggaacctctccttcctggacatctgctacaccacctcctccgttcccttagtccttgatggttttctgacccccaggaagacc
atctccttctcgggctgtgccgtgcagatgtttctctccttcgccatgggagccacagagtgtgtgctcctgggcatgatggcgtttgatc
gttatgtggccatctgcaacccccttagataccctgtggtcatgaacaaggctgcctatgtgcccatggccgtcagctcctgggtggctg
gcggtgctaactccttggtgcagatctcccttgcggtgcaattgcctttctgtggggacaatgtcatcaatcatttcatctgtgagatcctg
gcagtcttaaagctagcctgtgctgacatctccatcaatgtgatcagcatgggggtggccaatgtgattttcctgggggttccagttctgtt
catctttgtctcctacatcttcatactctccaccatcttgaggatcccctctgctgaggggaggaagaaggcAttctccacctgctctgcc
cacctcaccgtggtgatcattttttatgggaccatcctcttcatgtacgggaagcccaagtccaaggacccactgggggcagacaagca
ggaccttgcagacaagctcatctccctcttctatggactgctgacccccatgctgaaccccatcatctacagcctgaggaacaaggacg
tgaaggccgctgtgaggaacctggcaagtcacagatgcctcaccttctaa
(Olfr159, cDNA)
SEQ ID NO: 62
atggaAagatccaacaagaccacccctgtgtccagcttcatcctcctgggtctctccgcccacccaaagctggagaaaaccttcttcgt
gctcatcctgctgatgtacctggtgatcctgctgggcaatgtcgtcctcatcctggtgagcatcctcgactcccacctgcacacgcccat
gtacttcttcctggggaacctctccttcctggacatctgctacaccacctcctccgtccccctcattctggacagcttcctgacccccagg
aagaccatctccttctcgggctgtgccgtgcagatgtttctctccttcgccatgggagccacagagtgtgtgctcctgggcatgatggcg
tttgatcgttatgtggccatctgcaacccccttagatatcctgtggtcatgagcaaggctgcctatgtgcccatggccgccggctcctgg
gtatctggaagcatcactgccacagtgcagatatctttagcaatgacactgcccttctgtggggacaatgtcatcaatcactttacatgtga
gatcctggcagtcctgaaactggcctgtgctgacatctccatcaatgtcatcagcatggcagtggctaatgcaatgtttttgggggtccct
gtcctcttcatctttgtctcctacatcttcatactctccaccatcctgaggatcccctctgccgaggggaggaagaaggcAttctccacct
gctctgcccacctcaccgtggtacttgtcttctatgggaccatcctgttcatgtacgggaagcccaaatccaaggacccattgggggca
gacaaacaggaccttgcagacaagctcatctccctcttctatggagtggtgacccccatgctgaaccccatcatctacagcctgaggaa
caaggatgtgaaggccgctgtgacaaacctggtgggccagaaacacttcaagtggtga
(Olfr275, cDNA)
SEQ ID NO: 63
atggtAcagggaaattggacctctgtcacagtgtttgtttttctgggattttcgcactaccccagaattgaagtcaccgtatttgtgctgtgt
ctgctgatgtatctgatcaccttgctgggaaatactattctgatctccatcactatccttgattctcatctgcacactcccatgtactttttcc
tcagcaacctctcatttctggacatctggtatacctcttctgctctcactcctatgctggcaaactttgtttcagggaaaaacaccatttcctt
ctcgggatgtgcctcgcagatgtacttctctcttgccatgggctccactgaatgtgtgctcctctctatgatggcctatgacaggtatgtggcc
atctgtaacccactgagatatcccattattatgaacaggagggtctgtgtacagattgcaggcagctcctgggttacaggctgcctcactg
ccttggtggaaactggacctgtgattcatctgtctctctgtggtaacagcatcatcaatcatttcacctgtgaaattctggctctcttaaaatt
ggcctgtggagacacttccatggtacagttaattatgctggtgatcagcattcttcttctcccattgccaatgttgctcatttgtgtctcctat
gcatctatcctctccaacatcttgaggatcagctcaatggatggccgaagcaaagccttttcaacatgtgcagcccacttgactgtggtggt
tctgttctatgggacagctctctccatgtacctgaagccctcatctgtaaactcacaggaaattgataaatttatggcattaatatatactgg
actaacaccaatgctaaatcctattatctacagtctacggaacaaagaagtaaaaatggctgtgaaaaaattgttgatgaaaaaccccttc
agtgctatcttaacttctgtcctcaaataa
(OR10X1, cDNA)
SEQ ID NO: 64
atgaaAatcaaccagacaatcctgaaggaattcattcttgttggcttttctgtgtacccacatgtacagacatttctttttgtggtcttctttt
gtctctaccttctcacccttgcaggtaatctgatcatcatgggtctaacttgggtggacaggtccctccacacccctatgtatctcttccttagt
gcactctccttctctgagacctgctatacgctgaccatcgtccccaagatgctggaagatctactggccaaggacagaagcatttcagtc
acaggttgtagcttacagatgtgcttcttcttgggacttggtggcacaaactgtatcattctcactttgatgggatatgaccgcttcctggcc
atctgtaaccctctaagatatccactgcttatgaccaacattgtatgtggacaacttgtggcctctgcttgcactgcaggcttctttatctctct
tacagagactgcactgatattcagggactctttctgcagacccaaccttgtcaaacacttcttctgccatatgctggcagttattaggctgt
cttgtatagacagtaaccacacagaattcattataacactgatctcagtgtctggtttgctgggtacccttctgctcatcatcctgactgatgt
cttcattatttctactgtcctcaggatcccttcagctgagggcaagcagaaggcAttcaccacctgtgcctcccacctcaccgtggttata
atccactttggttttgcatctattgtttatttgaagccagaagcctcaggagatgacacactcatagcagtcccttatactgtcattaccccct
tcctcagccccatcatattcagcctgaggaataaggacatgaaaaatgcttttagaagaatgatgggaaacacagttgccttgaaaaaat
aa
(OR13J1, cDNA)
SEQ ID NO: 65
atggaAccgctcaacagaacagaggtgtccgagttctttctgaaaggattttctggctacccagccctggagcatctgctcttccctctg
tgctcagccatgtacctggtgaccctcctggggaacacagccatcatggcggtgagcgtgctagatatccacctgcacacgcccgtgt
acttcttcctgggcaacctctctaccctggacatctgctacacgcccacctttgtgcctctgatgctggtccacctcctgtcatcccggaa
gaccatctcctttgctgtctgtgccatccagatgtgtctgagcctgtccacgggctccacggagtgcctgctactggccatcacggccta
tgaccgctacctggccatctgccagccactcaggtaccGcgtgctcatgagccaccggctctgcgtgctgctgatgggagctgcctg
ggtcctctgcctcctcaagtcggtgactgagatggtcatctccatgaggctgcccttctgtggccaccacgtggtcagtcacttcacctg
caagatcctggcagtgctgaagctggcatgcggcaacacgtcggtcagcgaagacttcctgctggcgggctccatcctgctgctgcct
gtacccctggcattcatctgcctgtcctacttgctcatcctggccaccatcctgagggtgccctcggccgccaggtgctgcaaagccttc
tccacctgcttggcacacctggctgtagtgctgcttttctacggcaccatcatcttcatgtacttgaagcccaagagtaaggaagcccac
atctctgatgaggtcttcacagtcctctatgccatggtcacgaccatgctgaaccccaccatctacagcctgaggaacaaggaggtgaa
ggaggccgccaggaaggtgtggggcaggagtcgggcctccaggtga
(OR2S2, cDNA)
SEQ ID NO: 66
Atggaaaaagccaatgagacctcccctgtgatggggttcgttctcctgaggctctctgcccacccagagctggaaaagacattcttcgt
gctcatcctgctgatgtacctcgtgatcctgctgggcaatggggtcctcatcctggtgaccatccttgactcccgcctgcacacgcccat
gtacttcttcctagggaacctctccttcctggacatctgcttcactacctcctcagtcccactggtcctggacagctttttgactccccagga
aaccatctccttctcagcctgtgctgtgcagatggcactctcctttgccatggcaggaacagagtgcttgctcctgagcatgatggcattt
gatcgctatgtggccatctgcaacccccttaggtactccgtgatcatgagcaaggctgcctacatgcccatggctgccagctcctgggc
tattggtggtgctgcttccgtggtacacacatccttggcaattcagctgcccttctgtggagacaatgtcatcaaccacttcacctgtgaga
ttctggctgttctaaagttggcctgtgctgacatttccatcaatgtgatcagcatggaggtgacgaatgtgatcttcctaggagtcccggtt
ctgttcatctctttctcctatgtcttcatcatcaccaccatcctgaggatcccctcagctgaggggaggaaaaaggtcttctccacctgctct
gcccacctcaccgtggtgatcgtcttctacgggaccttattcttcatgtatgggaagcctaagtctaaggactccatgggagcagacaaa
gaggatctttcagacaaactcatcccccttttctatggggtggtgaccccgatgctcaaccccatcatctatagcctgaggaacaaggat
gtgaaggctgctgtgaggagactgctgagaccaaaaggcttcactcagtga
(OR13C3, cDNA)
SEQ ID NO: 67
atgggAgagattaaccagacacttgtgtcagaatttcttcttctgggtctttctggatacccaaagattgagattgtttactttgctctcattct
agttatgtacctagtgattctaattggcaatggtgttctaatcatagccagcatctttgattctcattttcacacaccaatgtacttcttcctgg
gcaacctctctttcctggatatctgctatacatcctcctctgttccctcaacattggtgagcttaatctcaaagaaaagaaacatttccttctct
ggatgtgcagtgcagatgttctttgggtttgcaatggggtcaacagaatgtctgcttcttggcatgatggcatttgatcgttatgtggccatct
gcaacccactgagataccccatcatcctgagcaaggtggcgtatgtattgatggcttctgtgtcctggctgtccggtggaataaattcag
ctgtgcaaacattacttgccatgagactgcctttctgtgggaataatattatcaatcatttcgcatgtgaaatattagctgtcctcaagctggc
ctgtgctgatatatccctcaatattatcaccatggtgatatcaaatatggccttcctggttcttccactgatggtcatttttttctcctatatgt
tcatcctctacaccatcttgcaaatgaattcagccacaggaagacgcaaggcattttccacgtgctcagctcacctgactgtggtgatcatat
tttacggtaccatcttctttatgtatgcgaaaccgaagtctcaagacctgattggggaagaaaaattgcaagcattagacaagctcatttct
ctgttttatggggtagtgacacccatgctgaatcctatactctatagcttgagaaataaggatgtaaaagctgctgtaaaatatttgctgaac
aaaaaaccaattcactaa
(OR13C4, cDNA)
SEQ ID NO: 68
atggacaagataaaccagacatttgtgagagaattcattcttctgggactctctggttaccccaaacttgagatcattttctttgctctgattc
tagttatgtacgtagtgattctaattggcaatggtgttctgatcatagcaagcatcttggattctcgtcttcacatgcccatgtacttcttcctg
ggcaacctctctttcctggatatctgctatacaacctcctccattccctcaacactggtgagcttaatctcaaagaaaagaaacatttccttc
tctggatgtgcagtgcagatgttctttgggtttgcaatggggtcaacagaatgtttcctccttggcatgatggcatttgatcgttatgtggcc
atctgtaaccctctgagataccccatcatcatgaacaaggtggtgtatgtactgctgacttctgtatcatggctttctggtggaatcaattca
actgtgcaaacatcacttgccatgcgatggcctttctgtgggaacaatattattaatcatttcttatgcgagatcttagctgtcctaaaattag
cttgttctgatatatctgtcaatattgttaccctagcagtgtcaaatattgctttcctagttcttcctctgctcgtgatttttttctcctatat
gttcatcctctacaccatcttgcgaacgaactcggccacaggaagacacaaggcattttctacatgctcagctcacctgactgtggtgatcatat
tttatggtaccatcttctttatgtatgcaaaacctaagtcccaggacctccttgggaaagacaacttgcaagctacagaggggcttgtttccat
gttttatggggttgtgacccccatgttaaaccccataatctatagcttgagaaataaagatgtaaaagctgctataaaatatttgctgagca
ggaaagctattaaccagtaa
(OR13C8, cDNA)
SEQ ID NO: 69
atggaaaggaccaacgattccacgtcgacagaatttttcctggtagggctttctgAccacccaaagctccagacagttttcttcgttctaa
ttttgtggatgtacctgatgatcctgcttggaaatggagtccttatctcagttatcatctttgattctcacctgcacacccccatgtatttctt
cctctgtaatctttccttcctcgacgtttgctacacaagttcctctgtcccactaattcttgccagctttctggcagtaaagaaaaaggtttcc
ttctctgggtgtatggtgcaaatgtttatttcttttgccatgggggccacggagtgcatgatcttaggcacgatggcactggaccgctatgtggc
catctgctacccactgagataccctgtcatcatgagcaagggtgcctatgtggccatggcagctgggtcctgggtcactgggcttgtgg
actcagtagtgcagacagcttttgcaatgcagttaccattctgtgctaataatgtcattaaacattttgtctgtgaaattctggctatcttgaaa
ctggcctgtgctgatatttcaatcaatgtgattagtatgacagggtcgaatctgattgttctggttattccattgttagtaatttccatctctta
catatttattgttgccactattctgaggattccttccactgaaggaaaacataaggActtctccacctgctcagcccacctgacagtggtgat
tatattctatggaaccatcttcttcatgtacgcaaagcctgagtctaaagcctctgttgattcaggtaatgaagacatcattgaggccctcat
ctcccttttctatggagtgatgactcccatgcttaatcctctcatctatagtctgcgaaacaaggatgtaaaggctgctgtcaaaaacatact
gtgtaggaaaaacttttctgatggaaaatga
(OR13C5, cDNA)
SEQ ID NO: 70
Atggaatgggaaaaccacaccattctggtggaattttttctgaagggactttctggtcacccaagacttgagttactcttttttgtgctcatc
ttcataatgtatgtggtcatccttctggggaatggtactctcattttaatcagcatcttggaccctcaccttcacacccctatgtacttctttc
tggggaacctctccttcttggacatctgctacaccaccacctctattccctccacgctagtgagcttcctttcagaaagaaagaccatttccct
ttctggctgtgcagtgcagatgttcctcagcttggccatggggacaacagagtgtgtgcttctgggcgtgatggcctttgaccgctatgt
ggctatctgcaaccctctgagatatcccatcatcatgagtaaggatgcctatgtacccatggcagctgggtcctggatcataggagctgt
caattctgcagtacaaacagtgtttgtggtacaattgcctttctgcaggaataacatcatcaatcatttcacctgtgaaattctagctgtcatg
aaactggcctgtgctgacatctcaggcaatgagttcatcctgcttgtgaccacaacattgttcctattgacacctttgttattaattattgtctc
ttacacgttaatcattttgagcatcttcaaaattagctcttcggaggggagaagcaaaccttcctctacctgctcagctcgtctgactgtggt
gataacattctgtgggaccatcttcctcatgtacatgaagcccaagtctcaagagacacttaattcagatgacttggatgccactgacaaa
cttatattcatattctacagggtgatgactcccatgatgaatcctttaatctacagtcttagaaacaaggatgtgaaggaggcagtaaaaca
cctactgagaagaaaaaattttaacaagtaa
(OR13C2, cDNA)
SEQ ID NO: 71
Atggaatgggaaaaccacaccattctggtggaattttttctgaagggactttctggtcacccaagacttgagttactcttttttgtgctcatc
ttcataatgtatgtggtcatccttctggggaatggtactctcattttaatcagcatcttggaccctcaccttcacacccctatgtacttctttc
tggggaacctctccttcttggacatctgctacaccaccacctctattccctccacgctagtgagcttcctttcagaaagaaagaccatttccct
ttctggctgtgcagtgcagatgttcctcggcttggccatggggacaacagagtgtgtgcttctgggcatgatggcctttgaccgctatgt
ggctatctgcaaccctctgagatatcccatcatcatgagtaaggatgcctatgtacccatggcagctgggtcctggatcataggagctgt
caattctgcagtacaatcagtgtttgtggtacaattgcctttctgcaggaataacatcatcaatcatttcacctgtgaaattctggctgtcatg
aaactggcctgtgctgacatctcagacaatgagttcatcatgcttgtggccacaacattgttcatattgacacctttgttattaatcattgtctc
ttacacgttaatcattgtgagcatcttcaaaattagctcttccgaggggagaagcaaagcttcctctacctgttcagcccatctgactgtgg
tcataatattctatgggaccatcctcttcatgtacatgaagcccaagtctaaagagacacttaattcggatgacttggatgctaccgacaaa
attatatccatgttctatggggtgatgactcccatgatgaatcctttaatctacagtcttagaaacaaggatgtgaaagaggcagtaaaaca
cctactgaacagaaggttctttagcaagtga
(OR13C9, cDNA)
SEQ ID NO: 72
Atggaatgggaaaaccaaaccattctggtggaattttttctgaagggacattctgttcacccaaggcttgagttactcttttttgtgctaatc
ttcataatgtatgtggtcatccttctggggaatggtactctcattttaatcagcatcttggaccctcaccttcacacccctatgtacttctttc
tggggaacctctccttcttggacatctgctacaccaccacctctattccctccacactagtgagcttcctttcagaaagaaagaccatttccttt
tctggctgtgcagtgcagatgttccttggcttggccatggggacaacagagtgtgtgcttctgggcatgatggcctttgaccgctatgtg
gctatctgcaaccctctgagatatcccatcatcatgagcaagaatgcctatgtacccatggctgttgggtcctggtttgcagggattgtca
actctgcagtacaaactacatttgtagtacaattgcctttctgcaggaagaatgtcatcaatcatttctcatgtgaaattctagctgtcatgaa
gttggcctgtgctgacatctcaggcaatgagttcctcatgcttgtggccacaatattgttcacattgatgccactgctcttgatagttatctct
tactcattaatcatttccagcatcctcaagattcactcctctgaggggagaagcaaagctttctctacctgctcagcccatctgactgtggt
cataatattctatgggaccatcctcttcatgtatatgaagcccaagtctaaagagacacttaattcagatgacttggatgctaccgacaaaa
ttatatccatgttctatggggtgatgactcccatgatgaatcctttaatctacagtcttagaaacaaggatgtgaaagaggcagtaaaacac
ctaccgaacagaaggttctttagcaagtga
(OR13D1, cDNA)
SEQ ID NO: 73
atggaAacaagaaattactctgccatgactgaattctttctggtggggctttcccaatatccagagctccagctttttctgttcctgctctgc
ctcatcatgtacatgataatcctcctgggaaatagcctcctcattatcatcaccatcttggattctcgcctccatactcccatgtatttctttc
ttggaaacctctcattcttggacatctgttacacatcctcatccattcctccaatgcttattatatttatgtctgagagaaaatccatctcctt
cattggctgtgctctgcagatggttgtgtcccttggcttgggctccactgagtgtgtcctcctggctgtgatggcctatgaccactatgtggcca
tctgcaacccactgaggtactccatcatcatgaacggagtgctgtatgtgcaaatggctgcatggtcctggatcataggctgtctgacct
ccctattgcaCacagttctgacaatgatgttgcctttctgtgggaataatgtcattgatcatattacctgtgaaattttggcccttctaaaactt
gtttgttcagatatcaccatcaatgtgcttatcatgacagtgacaaatattgtttcactggtgattcttctactgttaattttcatctcctatgt
gtttattctctcttccatcctgagaattaattgtgctgagggaagaaagaaagccttctctacctgttcagcgcactTgattgtggtcatcttat
tctacggttcagccctttttatgtacatgaaacccaagtcaaagaacactaatacatctgatgagattattgggctgtcttatggagtggtaag
cccaatgttaaatcccatcatctatagcctcaggaataaagaggtcaaagaggctgtaaagaaagtcctgagcagacatctgcatttatt
gaaaatgtga
(OR2K2, cDNA)
SEQ ID NO: 74
Atgcaaggagaaaacttcaccatttggagcatttttttcttggagggattttcccagtacccagggttagaagtggttctcttcgtcttcag
ccttgtaatgtatctgacaacgctcttgggcaacagcactcttattttgatcactatcctagattcacgccttaaaacccccatgtacttattc
cttggaaatctctctttcatggatatttgttacacatctgcctctgttcctactttgctggtgaacttgctgtcatcccagaaaaccattatct
tttctgggtgtgctgtacagatgtatctgtcccttgccatgggctccacagagtgtgtgctcctggccgtgatggcatatgaccgttatgtggc
catttgtaacccgctgagatactccatcatcatgaacaggtgcgtctgtgcacggatggctacggtctcctgggtgacgggttgcctgac
cgctctgctggaaaccagttttgccctgcagatacccctctgtgggaatctcatcgatcacttcacgtgtgaaattctggcggtgctaaag
ttagcttgcacaagttcactgctcatgaacaccatcatgctggtggtcagcattctcctcttgccaattccaatgctcttagtttgcatctctta
catcttcatcctttccactattctgagaatcacctcagcagagggaagaaacaaggctttttctacctgtggtgcccatttgactgtggtgat
tttgtattatggggctgccctctctatgtacctaaagccttcttcatcaaatgcacaaaaaatagacaaaatcatctcgttgctttacggagt
gcttacccctatgttgaaccccataatttacagtttaagaaacaaggaagtcaaagatgctatgaagaaattgctgggcaaaataacattg
catcaaacacacgaacatctctga
(OR9A4, cDNA)
SEQ ID NO: 75
atgttAatgaattactctagtgccactgaattttatctccttggcttccctggctctgaagaactacatcatatcctttttgctatattcttct
ttttctacttggtgacattaatgggaaacacagtcatcatcatgattgtctgtgtggataaacgtctgcagtcccccatgtatttcttcctcgg
ccacctctctgccctggagatcctggtcacaaccataatcgtccccgtgatgctttggggattgctgctccctgggatgcagacaatatatttgt
ctgcctgtgttgtccagctcttcttgtaccttgctgtggggacaacagagttcgcattacttggagcaatggctgtggaccgttatgtggct
gtctgtaaccctctgaggtacaacatcattatgaacagacacacctgcaactttgtggttcttgtgtcatgggtgtttgggtttctttttcaaat
ctggccggtctatgtcatgtttcagcttacttactgcaaatcaaatgtggtgaacaattttttttgtgaccgagggcaattgctcaaactatcc
tgcaataatactcttttcacggagtttatcctcttcttaatggctgtttttgttctctttggttctttgatccctacaattgtctccaacgccta
catcatctccaccattctcaagatcccgtcatcctctggccggaggaaatccttctccacttgtgcctcccacttcacctgtgttgtgattggc
tacggcagctgcttgtttctctacgtgaaacccaagcaaacgcaggcagctgattacaattgggtagtttccctgatggtttcagtagtaactc
ctttcctcaatcctttcatcttcaccctccggaatgataaagtcatagaggcccttcgggatggggtgaaacgctgctgtcaactattcag
gaatta
(OR9A2, cDNA)
SEQ ID NO: 76
Atgatggacaaccactctagtgccactgaattccaccttctaggcttccctgggtcccaaggactacaccacattctttttgctatattcttt
ttcttctatttagtgacattaatgggaaacacggtcatcattgtgattgtctgtgtggataaacgtctgcagtcccccatgtatttcttcctcag
ccacctctctaccctggagatcctggtcacaaccataattgtccccatgatgctttggggattgctcttcctgggatgcagacagtatcttt
ctctacatgtatcgctcaacttttcctgtgggaccatggagtttgcattacttggagtgatggctgtggaccgttatgtggctgtgtgtaacc
ctttgaggtacaacatcattatgaacagcagtacctgtatttgggtggtaatagtgtcatgggtgtttggatttctttctgaaatctggcccat
ctatgccacatttcagtttaccttccgcaaatcaaattcattagaccatttttactgtgaccgagggcaattgctcaaactgtcctgcgataa
cactcttctcacagagtttatccttttcttaatggctgtttttattctcattggttctttgatccctacgattgtctcctacacctacattatc
tccaccatcctcaagatcccgtcagcctctggccggaggaaagccttctccacttttgcctcccacttcacctgtgttgtgattggctatggca
gctgcttgtttctctacgtgaaacccaagcaaacacagggagttgagtacaataagatagtttccctgttggtttctgtgttaacccccttcctg
aatcctttcatctttactcttcggaatgacaaagtcaaagaggccctccgagatgggatgaaacgctgctgtcaactcctgaaagattag
(OR6X1, cDNA)
SEQ ID NO: 77
atgagaaatggcacagtaatcacagaattcatcctgctaggctttcctgttatccaaggActacaaacacctctctttattgcaatctttctc
acctacatattaacccttgcaggcaatgggcttattattgccactgtgtgggctgagcccaggctacaaattccaatgtacttcttcctttgt
aacttgtctttcttagaaatctggtacaccaccacagtcatccccaaactgctaggaacctttgtagtggcaagaacagtaatctgcatgtc
ctgctgcctgctgcaggcAttcttccacttcttcgtgggcaccaccgagttcttgatcctcactatcatgtcttttgaccgctacctcaccat
ctgcaatccccttcaccaccccaccatcatgaccagcaaactctgcctgcagctggccctgagctcctgggtggtgggcttcaccattg
tcttttgtcagacgatgctgctcatccagttgccattctgtggcaataatgttatcagtcatttctactgtgatgttgggcccagtttgaaagc
cgcctgcatagacaccagcattttggaactcctgggcgtcatagcaaccatccttgtgatcccagggtcacttctctttaatatgatttctta
tatctacattctgtccgcaatcctacgaattccttcagccactggccaccaaaagactttctctacctgtgcctcgcacctgacagttgtctc
cctgctctacggggctgttctgttcatgtacctaagacccacagcacactcctcctttaagattaataaggtggtgtctgtgctaaatactat
cctcaccccccttctgaatccctttatttatactattagaaacaaggaggtgaagggagccttaagaaaggcaatgacttgcccaaagac
tggtcatgcaaagtaa
(OR9K2, cDNA)
SEQ ID NO: 78
atgggAgacaggggaacaagcaatcactcagaaatgactgacttcattcttgcaggcttcagggtacgcccagagctccacattctcc
tcttcctgctatttttgtttgtttatgccatgatccttctagggaatgttgggatgatgaccattattatgactgatcctcggctgaacacacc
aatgtattttttcctaggcaatctctccttcattgatcttttctattcatctgttattgaacccaaggctatgatcaacttctggtctgaaaac
aagtctatctcctttgcaggctgtgtggcccagctctttctctttgccctcctcattgtgactgagggatttctcctggcggccatggcttatg
accgctttattgccatctgcaaccctctgctctactctgttcaaatgtccacacgtctgtgtactcagttggtggctggttcctatttttgtgg
ctgcattagctcagttattcagactagcatgacatttactttatctttttgcgcttctcgggctgttgaccacttttactgtgattctcgccca
cttcagagactgtcttgttctgatctctttatccatagaatgatatctttttccttatcatgtattattatcttgcctactatcatagtcatta
tagtatcttacatgtatattgtgtccacagttctaaagatacattctactgagggacataagaaAgccttctccacctgcagctctcacctggg
agttgtgagtgtgctgtatggtgctgtcttttttatgtatctcactcctgacagatttcctgagctgagtaaagtggcatccttatgttactcc
ctagtcactcccatgttgaatcctttgatttactctctgaggaacaaagatgtccaagaggctctaaaaaaatttctagagaagaaaaatatta
ttctttga
(OR13F1, cDNA)
SEQ ID NO: 79
atgttcccggcaaattggacatctgtaaaagtatttttcttcctgggattttCtcactaccccaaagttcaggtcatcatatttgcggtgtgctt
gctgatgtacctgatcaccttgctgggcaacatttttctgatctccatcaccattctagattcccacctgcacacccctatgtacctcttcctc
agcaatctctcctttctggacatctggtactcctcttctgccctctctccaatgctggcaaactttgtttcagggagaaacactatttcattctc
agggtgcgccactcagGtgtacctctcccttgccatgggctccactgagtgtgtgctcctgcccatgatggcatatgaccggtatgtgg
ccatctgcaaccccctgagataccctAtcatcatgaataggagaacctgtgtgcagattgcagctggctcctggatgacaggctgtctc
actgccatggtggaaatgatgtctgtgctgccactgtctctctgtggtaatagcatcatcaatcatttcacttgtgaaattctggccatcttga
aattggtttgtgtggacacctccctggtgcagttaatcatgctggtgatcagtgtacttcttctccccatgccaatgctactcatttgtatctct
tatgcatttatcctcgccagtatcctgagaatcagctcagtggaaggtcgaagtaaagccttttcaacgtgcacagcccacctgatggtg
gtagttttgttctatgggaTggctctctccatgcacctgaagccctccgctgtagattcacaggaaatagacaaatttatggctttggtgta
tgccggacaaacccccatgttgaatcctatcatctatagtctacggaacaaagaggtgaaagtggccttgaaaaaattgctgattagaaa
tcattttaatactgccttcatttccatcctcaaataa
(LOC500460, cDNA)
SEQ ID NO: 80
atggtAcagggaaattggtcctctgtcacagtgtttttcttcctgggattttctcagtaccccagaattgaagtcaccatatttgtgctgtgtc
tgctgatgtacctgatcaccttgctgggaaatactattctgatctccatcactatccttgattctcacctgcacactcccatgtactttttcctt
agcaacctctcctttctggacatctggtatacctcttctgctctcactcctatgctggcaaactttgtttcaggggaaaacaccatctcattctc
aggatgtgcttttcagatgtacttctctcttgccatgggctccacggaatgtgtgctcctctctatgatggcctatgacaggtacgtggcca
tctgcaaccccctgagatatcccatcattatgaacaggagagtctgtgtacagattgcaggcagctcctgggttacaggctgcctcactg
ccttggtggaaactggacctgtgattcatctgtctctctgtggtaataacatcatcaatcatttcacctgtgaaattctggctctcttaaaaat
ggcttgtggagacactgccatggtgcagttaattatgttagtgatcagcatccttcttctcccattgccaatgttgctcatttgtgtctcctatg
catccatcctctccaacatcttgaggatcagctcagtggatggccgaagcaaagccttttcaacatgtgcagcccacttgactgtggtgg
ttctgttctatgagacagctctctccatgtacctgaagcccttatctgtaaactcacaggaaatagataaatttatggcattgatatatgttgg
actaacaccaatgctaaatcctattatctacagtctacggaacaaagaagtcaaaatggctgtgaaaaaattgttgattaaaaatcccttca
ctgctatcttaacttctgtcctcaaataa
SEQ ID NO: 81
AACTTTTTAATGA
SEQ ID NO: 82
ACATAACTTTTTAATGAGTCT
SEQ ID NO: 83
YTTTTAATGAR
where Y = C/T and R = A/G
SEQ ID NO: 84
TAATGA

We claim:

1. A biosensor comprising:
one or more populations of olfactory sensory neurons (OSNs), or cilia derived therefrom;
wherein each population of OSNs preferentially expresses an odorant receptor (OR) comprising an amino acid sequence selected from the group consisting of SEQ ID NOs: 1-40, or an amino acid sequence with at least 85% identity to any one of SEQ ID NOs: 1-40.

2. The biosensor of claim 1, wherein the OR comprises an amino acid sequence selected from the group consisting of SEQ ID NOs: 1, and 6-18, or an amino acid sequence with at least 85% identity to any one of SEQ ID NOs: 1, or 6-18.

3. The biosensor of claim 1 or 2, wherein the one or more populations of OSNs, or cilia derived therefrom, are attached to a solid support.

4. The biosensor of claim 3, wherein the solid support is selected from the group consisting of silicon, glass, polystyrene, and polymers.

5. The biosensor of any one of claims 1 to 4, wherein the one or more populations of OSNs further express one or more markers for detecting activation or lack of activation of the OR.

6. The biosensor of claim 5, wherein the marker is a calcium-sensitive fluorescent dye selected from the group consisting of fura-2, fluo-3, fluo-4, fluo-5F, indo-1, and Oregon Green BAPTA.

7. The biosensor of claim 5, wherein the marker is selected from the group consisting of GECO2.1, GCaMP6, Flamindo, Flamindo2, and Pink Flamindo.

8. The biosensor of any one of claims 5-7, wherein the marker for detecting activation or lack of activation of the OR is co-expressed with the preferentially expressed odorant receptor (OR).

9. The biosensor of any one of claims 1-8, wherein the OSNs comprise an enhancer operably linked to the sequence encoding the preferentially expressed OR.

10. The biosensor of claim 9, wherein the enhancer comprises at least four sequential repeats of a 21 base pair (bp) sequence wherein each 21 bp sequential repeat comprises the sequence AACTTTTTAATGA (SEQ ID NO:81).

11. The biosensor of claim 10, wherein the enhancer comprises at least four sequential repeats of ACATAACTTTTTAATGAGTCT (SEQ ID NO: 82).

12. The biosensor of claim 10 or claim 11, wherein the enhancer comprises ten or fewer of the 21 bp sequential repeats.

13. The biosensor of claim 9, wherein the enhancer comprises one or more TetO sequences.

14. A biosensor comprising:
a cell or population of cells engineered to express an OR comprising an amino acid sequence selected from the group consisting of SEQ ID NOs: 1-40, or an amino acid sequence with greater than 85% identity to any one of SEQ ID NOs: 1-40.

15. The biosensor of claim 14, wherein the OR comprises an amino acid sequence selected from the group consisting of SEQ ID NOs: 1, and 6-18, or an amino acid sequence with at least 85% identity to any one of SEQ ID NOs: 1, or 6-18.

16. The biosensor of claim 14 or 15, wherein the cell is a eukaryotic cell or population of cells is a population of eukaryotic cells.

17. The biosensor of claim 16, wherein the eukaryotic cell is selected from the group consisting of yeast, and an olfactory sensory neuron.

18. The biosensor according to any one of claims 14-17, wherein the cell or population of cells further expresses one or more markers for detecting activation or lack of activation of the OR.

19. The biosensor of claim 18, wherein the marker is a calcium-sensitive fluorescent dye selected from the group consisting of fura-2, fluo-3, fluo-4, fluo-5F, indo-1, and Oregon Green BAPTA.

20. The biosensor of claim 18, wherein the marker is selected from the group consisting of GECO2.1, GCaMP6, Flamindo, Flamindo2, and Pink Flamindo.

21. The biosensor of any one of claims 14-20, wherein the marker for detecting activation or lack of activation of the OR is co-expressed with the expressed OR.

22. A transgenic animal comprising an olfactory epithelium in which the OSNs preferentially express an OR comprising an amino acid sequence selected from the group consisting of SEQ ID NOs: 1-40, or an amino acid sequence with greater than 85% identity to any one of SEQ ID NOs: 1-40.

23. A transgenic animal comprising:
a) a transgene sequence encoding an OR comprising an amino acid sequence selected from the group consisting of SEQ ID NOs: 1-40, or an amino acid sequence with greater than 85% identity to any one of SEQ ID NOs: 1-40; and
b) an enhancer operably linked to the transgene sequence.

24. A transgenic animal of claim 22 or 23, wherein the OR comprises an amino acid sequence selected from the group consisting of SEQ ID NOs: 1, and 6-18, or an amino acid sequence with at least 85% identity to any one of SEQ ID NOs: 1, or 6-18.

25. The transgenic animal of claim 23 or 24, wherein the enhancer comprises at least four sequential repeats of a 21 bp sequence wherein each 21 bp sequential repeat comprises the sequence AACTTTTTAATGA (SEQ ID NO:81).

26. The transgenic animal of claim 23 or 24, wherein the enhancer comprises at least four sequential repeats of ACATAACTTTTTAATGAGTCT (SEQ ID NO:82).

27. The transgenic animal of claim 25 or 26, wherein the enhancer comprises ten or fewer of the 21 bp sequential repeats.

28. The transgenic animal of claim 23, wherein the enhancer comprises one or more TetO sequences.

29. The transgenic animal of any one of claims 22-28, wherein the transgenic animal is a non-human mammal.

30. The transgenic animal of claim 29, wherein the non-human mammal belongs to the family of Bovidae, Canidae, and Muridae.

31. The transgenic animal of claim 29, wherein the non-human mammal is rat, a mouse, a dog, cat, goat, chicken, sheep, pig, or primate.

32. A tissue isolated from the transgenic animal of any one of claims 22-31.

33. The tissue of claim 32, wherein the tissue is an olfactory epithelium.

34. A cell isolated from the transgenic animal of any one of claims 22-31.

35. An isolated cell or population of cells, wherein the cell or the or population of cells is engineered to express an OR comprising (i) an amino acid sequence selected from the group consisting of SEQ ID NOs: 1-40 or (ii) an amino acid sequence with greater than 85% identity to any one of SEQ ID NOs: 1-40.

36. The cell or population of cells of claim 35, wherein the OR comprises an amino acid sequence selected from the group consisting of SEQ ID NOs: 1, and 6-18, or an amino acid sequence with at least 85% identity to any one of SEQ ID NOs: 1, or 6-18.

37. The cell or population of cells of claim 35 or 36, wherein the cell is a eukaryotic cell or the population of cells is a population of eukaryotic cells.

38. The cell or population of cells of claim 37, wherein the eukaryotic cell is an olfactory sensory neuron or the population of eukaryotic cells is a population of OSNs.

39. The cell or population of cells of any one of claims 35-38, wherein cell or the population of cells further expresses one or more markers for detecting activation or lack of activation of the OR.

40. The cell or population of cells of claim 39, wherein the marker is a calcium-sensitive fluorescent dye selected from the group consisting of fura-2, fluo-3, fluo-4, fluo-5F, indo-1, and Oregon Green BAPTA.

41. The cell or population of cells of claim 39, wherein the marker is selected from the group consisting of GECO2.1, GCaMP6, Flamindo, and Flamindo2.

42. The cell or population of cells of any one of claims 35-41, wherein the marker for detecting activation or lack of activation of the OR is co-expressed with the preferentially expressed OR.

43. An expression construct comprising:
a. an OR coding sequence, wherein the OR coding sequence encodes an amino acid sequence selected from the group consisting of SEQ ID NOs: 1-40, or an amino acid sequence with greater than 85% identity to any one of SEQ ID NOs: 1-40; and
b. an enhancer operably linked to the OR coding sequence.

44. The expression construct of claim 43, wherein the OR comprises an amino acid sequence selected from the group consisting of SEQ ID NOs: 1, and 6-18, or an amino acid sequence with at least 85% identity to any one of SEQ ID NOs: 1, or 6-18.

45. The expression construct of claim 43 or 44, wherein the enhancer comprises at least four sequential repeats of a 21 bp sequence wherein each 21 bp sequential repeat comprises the sequence of AACTTTTTAATGA (SEQ ID NO:81).

46. The expression construct of claim 43 or 44, wherein the enhancer comprises at least four sequential repeats of ACATAACTTTTTAATGAGTCT (SEQ ID NO: 82).

47. The expression construct of claim 45 or 46, wherein the enhancer comprises ten or fewer of the 21 bp sequential repeats.

48. The expression construct of claim 43 or 44, wherein the enhancer comprises one or more TetO sequences.

49. The expression construct of claim 48, wherein the vector comprises a nucleic acid sequence encoding a tTA or an rtTA protein.

50. The expression construct of claim 49, wherein the rTA or rtTA protein comprises a sequence derived from VP16, VP32, VP48, VP64, or GAL4-VP16.

51. The expression construct of claim 48, wherein the one or more TetO sequences are upstream of a minimal CMV promoter.

52. The biosensor of any one of claims 1-21, the transgenic animal of any one of claims 22-30, the tissue of any one of claim 32 or 33, the cell of claim 34, the cell or population of cells of any of claims 35-42, or the expression construct of any of one claims 43-51, wherein the OR is differentially activated by one or more odorants associated with a change in the levels of one or more neurotransmitters in the central nervous system (CNS) of a subject, as compared to the levels for the one or more neurotransmitters in the CNS of a control subject, are present in the sebum, urine, or saliva of the subject and/or present in the sebum, urine, or saliva of the control subject.

53. The biosensor, transgenic animal, tissue, cell, cell or population of cells, or expression of claim 52, wherein the neurotransmitter is a catecholamine.

54. The biosensor, transgenic animal, tissue, cell, cell or population of cells, or expression construct of claim 52, wherein the neurotransmitter is selected from the group consisting of dopamine, norepinephrine (noradrenaline), epinephrine (adrenaline), histamine, and serotonin.

55. The biosensor, transgenic animal, tissue, cell, cell or population of cells, or expression construct of claim 54, wherein the neurotransmitter is dopamine.

56. The biosensor, transgenic animal, tissue, cell, cell or population of cells, or expression construct of any one of claims 52-55, wherein the subject has a disease or condition associated with a change in the levels of one or more neurotransmitters in the CNS as compared to the control levels for the one or more neurotransmitters.

57. The biosensor, transgenic animal, tissue, cell, cell or population of cells, or expression construct of any one of claims 52-56, wherein the subject has a disease or condition associated with a dopamine deficiency in the central nervous system.

58. The biosensor, transgenic animal, tissue, cell, or expression construct of claim 57, wherein the disease or condition associated with dopamine deficiency is Parkinson's disease (PD), depression, schizophrenia, dystonia, or restless leg syndrome.

59. The biosensor, transgenic animal, tissue, cell, or expression construct of claim 58, wherein the disease or condition associated with dopamine deficiency is PD.

60. A method for detecting a change in the levels of one or more neurotransmitters in the CNS of a subject as compared to control levels for the one or more neurotransmitters, the method comprising:
a. obtaining a sample from the subject;
b. exposing a biosensor according to any one of claim 1-21 or 52-59 to the sample or to an extract from the sample; and
c. measuring the activation or lack of activation of the one or more ORs by one or more odorant molecules in the sample obtained from said subject.

61. The method of claim 60, wherein measuring of the activation or lack of activation of the OR comprises detecting a decrease in ATP levels or a change in action potential.

62. The method of claim 60, wherein measuring of the activation or lack of activation of the OR comprises detecting an increase in Ca²⁺, GDP and/or cAMP levels.

63. The method of any one of claims 60-62, wherein the neurotransmitter is a catecholamine.

64. The method of any one of claims 60-62, wherein the neurotransmitter is selected from the group consisting of dopamine, norepinephrine (noradrenaline), epinephrine (adrenaline), histamine, and serotonin.

65. The method of any one of claims 60-64, wherein the subject has a disease or condition associated with a change in the levels of one or more neurotransmitters in the CNS as compared to the control levels the one or more neurotransmitters.

66. The method of claim 65, wherein the subject has a disease or condition associated with a dopamine deficiency in the central nervous system.

67. The method of claim 66, wherein the disease or condition associated with dopamine deficiency is PD, depression, schizophrenia, dystonia, or restless leg syndrome.

68. The method of claim 67, wherein the disease or condition associated with dopamine deficiency is PD.


SEQUENCE LISTING (83 sequences)

SEQ ID NO: 1 - protein, 319 aa, Rattus norvegicus
MDRSNETSLVSYFILLGLSAHPRLEKTFFVLILLMYLVILLGNGVLILVTILDSHLHTPMYFFLGNLSFLDICYTTSSIPLVLDGFLTPRKTISFSGCAMQMFLSFAMGATECVLLGMMAFDRYVAICNPLRYPVVMSKAAYVPMAAGSWAGGSITATVQTSLAMRLPFCGDNVINHFTCEILAVLKLACADISINVISMAVGNAVFLGVPVLFIFVSYIFILSTILRIPSAEGRKKAFSTCSAHLTVVIVFYGTILFMYGKPKSKDPLGADKQDLADKLISLFYGVLTPMLNPIIYSLRNKDVRAAVRNLLHQKHFKW

SEQ ID NO: 2 - protein, 315 aa, Rattus norvegicus
MESSNQTSPVSRFILLGLSAHPTLEKTFFVLILLMYLVILLGNGVLILVTILDSHLHTPMYFFLGNLSFLDICYTTSSVPLILDSFLTPRKTISFSGCAVQMFLSFAMGATECVLLSMMAFDRYVAICNPLRYPVVMSKAVYVPMATGSWAAGIAASLVQTSLAMRLPFCGDNVINHFTCEILAVLKLACADISINIISMGVTNVIFLGVPVLFISFSYIFILSTILRIPSAEGRKKAFSTCSAHLTVVIVFYGTILFMYGKPKSKDPLGADKQDLADKLISLFYGVLTPMLNPIIYSLRNKDVRAAVRNLVVLK

SEQ ID NO: 3 - protein, 318 aa, Rattus norvegicus
MEIANQSTVAEFVLLGLSDHPMLEKTFFVLILLMYLVILLGNGVLILVTILDSHLHTPMYFFLGNLSFLDICYTTSSIPLVLDGFLTPRKTISFSGCAMQMFLSFAMGATECVLLGMMAFDRYVAICNPLRYPVVMSKAAYVPMAVSSWVAGGANSMVQISLAVQLPFCGDNIINHFTCEILAVLKLACADISINVISMGVANVIFLGVPVLFIFVSYIFILSTILRIPSAEGRKKAFSTCSAHLTVVIVFYGTILFMYGKPKSKDPLGADKQDLADKLISLFYGVLTPMLNPIIYSLRNKDVKTAVRDLVGRKCLIQ

SEQ ID NO: 4 - protein, 318 aa, Rattus norvegicus
MEVSNQSTVTEFVLLGLSAHPTLEKTFFVLILLMYLVILLGNGVLILVTILDSHLHTPMYFFLGNLSFLDICYTTSSVPLVLDGFLTPRKTISFSGCAVQMFLSFAMGATECVLLGMMAFDRYVAICNPLRYPVVMSKAAYVPMAVSSWVAGGANSMVQISLAVQLPFCGDNVINHFICEILAVLKLACADISINVISMGVANVIFLGVPVLFIFVSYIFILSTILRIPSAEGRKKAFSTCSAHLTVVIIFYGTILFMYGKPKSKDPLGADKQDLADKLISLFYGLLTPMLNPIIYSLRNKDVKTAVRDLASHRCLTQ

SEQ ID NO: 5 - protein, 318 aa, Rattus norvegicus; identical to SEQ ID NO: 4

SEQ ID NO: 6 - protein, 319 aa, Rattus norvegicus
MVQGNWSSVTVFFFLGFSQYPRIEVTIFVLCLLMYLITLLGNTILISITILDSHLHTPMYFFLSNLSFLDIWYTSSALTPMLANFVSGENTISFSGCALQMYFSLAMGSTECVLLSMMAYDRYVAICNPLRYPIIMNRRVCVQIASSSWATGCLTALVETGPVIHLSLCGNNIINHFTCEILALLKMACGDTAMVQLIMLVISILLLPLPMLLICVSYASILSNILRISSVDGRSKAFSTCAAHLTVVVLFYGTALSMYLKPSSVNSQEIDKFMALIYAGLTPMLNPIIYSLRNKEVKMAVKKLLIRNPFSTILTSVLK

SEQ ID NO: 7 - protein, 307 aa, Rattus norvegicus
MAPINQSVVTMFFLQNFVDDPWIQNVLFCFFFALFVAAIAGNGLIITVIHSSANLHTPMYFFLVNLSLMDVICTVTVLPKVLQSLVAENAISYGGCLTQMFVFSWVLGSELLLFSAMAYDRYLAICRPLHYGTLMSGRVCIALATFVWFTGALNSLVLTCLVLPLSFCGPNLITHFFCEIPSVLMLSCSPTFINDIMTVIADMFLTGLNFLLTMTSYGFIIASILRIRSAEGKKRAFSTCSAHLVVVTLYYSTVLYTYVRPALGTSGLLDKVIAVLYTTVTPSLNPLIYTLRNKEFKTSFKKLLFPN

SEQ ID NO: 8 - protein, 311 aa, Rattus norvegicus
MDSFNATLEERFILVGFSDWPQLELILFVFISIVYSLTLFGNTAIIALSRMDLRLHTPMYFFLSHLSFLDLCYTTSTVPQLLINLHGLDRIISYGRCVAQLFISLALGSTECVLLVVMAFDRYAAVCRPLHYTTIMHPVLCQALAIASWVGGFLNSLIQTGLMMTIPLCGRRLNHFFCEMPVFLKLACKDTGDTEAKMFVARAIILVFPATLILGSYGHIARAVLKVKSMAGRRKAFGTCGSHLLVVSLFYGSAIYTYLQPKGSYSESDGKFVALFYTIVTPMLNPLIYTLRNKDVKGALWKVLGRSTGQE

SEQ ID NO: 9 - protein, 305 aa, Rattus norvegicus
MMNGTLVTEFFILGFSEMPHLRVPLLLSFFCLYMAAISGNLLIMVTISASPALHTPMYFFLVNLAMVDILCTSTILPKLLDSMMAGRSISYGGCMAQLFFFTWSLGAELLLFSAMAYDRFVAICCPLHYSAWMGPRVCAFLAGLVWSISITNTSVHTGLMLRLPFCGSNEIEHFFCEIPPLLKLSCAPTQLNEAMAFAADVFLAVGNFSVTILSYGFIVVSILRIRSAEGKRRAFSTCSAHLIVVTMYYSTVIYTYIRPASSYSLNKDKVVSIIYTSVAPTLNPLIYTLRNKDVKVALRRLLSCS

SEQ ID NO: 10 - protein, 317 aa, Rattus norvegicus
MDSPSNSTISRGFLLQGFFEFPHLRPLLFLVLLAVHLATLSGNLLILVAVVSVPSRPPMLLFLCQLSAIELCYTLVVVPRSLADLSMPGHGRGSPISFLGCAVQMQMFVALGGAECFLLAAMAYDRYVAICHPLRYASIVTPGLCARLALACCLGGLAVSVGLTVAVFHLPFCGSRLLVHFFCDITALLHLACTRSYIDELPLLGACLVLLLLPSMLILTSYGAIASALRRLHGGGGRRKAASTCASHLAVTFLHYGCATFMYVRPKSSYSPRLDRTLALVYTNVTPLLYPLIYSLRNREITSAIHRVLGLRGSGSM

SEQ ID NO: 11 - protein, 308 aa, Rattus norvegicus
MGYGNITEFILLGLFHDEDVKAICAVLFLLCYLAILCGNLIVLLTIKGSQLSEQPMYFFLSYLSFMDVCFTSTVAPKFIIGLLVQCNTISYNACIAQMFYAHFFGATEIFILVVMAYDRYVAICRPLYYMITMSRQVCYILVIGSVFGAFIHSLVHVLVIIRLPFCGTNEIDHYFCDIFPLLKLACTETRLLVIVIITTTGVMSILTFVALVISYIIILSILRTRSSEGRRKALSTCGSHITVVFMFFLPLIFTYVPMGDSVGDDKVFALFYTMIAPLFNPLIYTLRNTDMKNAMRKVWCQDKLFEGK

SEQ ID NO: 12 - protein, 312 aa, Rattus norvegicus
MNEINYTKVSEFVFLGLSKSKEIQHFFLAFSVVLYVAIVLGNTLVVFTLALDPHLHSPMYFLLGNLSFIDLCFSTLTVPKMISDLSSGHNTISFHGCVFQIFVIHVLGASEMVLLVVMAWDRYVAICKPLHYLTIMNPRICILLLSGAWIIAFLHSVIQLGFVVHLRFCGPNEIDSFYCDLPRFIKLACMDNYRMEFMVAANSGIISIGTFFLLIISYIVILLTVRKHSSGDLSKALSTLSAHISVVVLFFGPCIFVYMWPFPTVPVDKFLAILDFMITPILNPAIYTLRNKDMKVAMRKLSVQLLHFRKMS

SEQ ID NO: 13 - protein, 308 aa, Rattus norvegicus
MSLSDNPELQPILFVLFLSFYMVTVLGNLLIILAVSSDFHLHTPMYFFLSNLSWSDICLISTTVPKMIWDIRTQSRVISYAGCLTQMSMFIIFGCMDSMLLTVMAYDRFVAICHPLNYNIIMNPNLCALLLLASVLASLVDSQVHNLIVLQFTYFNDMEISNFFCEPSQLLNLKCSEMFTKNIVIRFIGVFFGLFSTSGIIFSYYKIVSSILRIPSTEGKYKAFSTCVSHLSVVCLFYGTAIGVYIGSTASNSSENCKVASLMYTVVTPMLNPFIYSLRNRDIKTALWKLQKKAKLHNAFHHFLILSS

SEQ ID NO: 14 - protein, 313 aa, Rattus norvegicus
MSSTNHSSVSVFLLLGLSRQPQQQQLLFLLFLIMYLATVLGNLLIILSISTDSRLHTPMYFFLSNLSFVDLCFSSTTVPKVLTNHILGSQEISFSGCLTQMYFLSVFADMDNFLLAVMAYDRFVAICHPLHYTKKMTHQLCALLVVESWLAVNLNALLHTLLMARLSFCGDNLIPHYFCDATPLLKLSCSDTHLNELMINTEGAVTMVTPFVCILISYIHITCAVLRVSSPRGGWKAFSTCGSHLAVVCLFYGTIIAVYFNPSTAHSPEKDTVATVLYTVVTPMLNPFIYSLRNRDLKWSLRKLVHRSTFSFQ

SEQ ID NO: 15 - protein, 315 aa, Rattus norvegicus
MMKANHSLEFILIGFTDHPDLKTLLFLVFSAIYLVTMVGNLGLVALIYMEPRFHTPMYIFLGNLALMDSCCSCAITPKMLENFFSVDRRISFCECMAQFYFLCLAETADCFLLAAMAYDRYVAICNPLQYHTMMSKKLSLQMSMGIFIASNLHSLIHTGCLLRLNFCKSTKIDHFFCDILPLYRLSCTNPFINELMIYIFSMPIQVFTITTILVSYFCILFTIFKMKSKDGRGKAFSTCASHFFSVSIFYTCLLMYIGPSEDGSKDIPVAVFYTVIIPLLNPFIYSLRNKEVVNAVKKVMKTYSIFKNASASIVH

SEQ ID NO: 16 - protein, 316 aa, Rattus norvegicus
MVPMERNVSVEIIFVLVGFTDYPELQIPLFLVFLFMYIITVVGNLGMIVLINIDPKFHTPMYFFLSHLSFVDFCYPTIIMPKLLENLILADKTILYFSCMLQYFLSCVAVVAESYLLAVMAYDRFVAICNPLLYTVAMSQRLCILLVTGSYIWSTLATLILLCYALQLKFSRFTVINHFFCEYTALIAVSSSDIHIPSLLLFCFATFNEVSTLLIILTSYVFIFVTVLKIKSASGRRKAFSTCASHLTAITIFHGTILSLYCVPNPQNSRNAVKVASVFYAVVNPLLNPLIYSLRNKDVKEVLQKVVSTSLKFPLH

SEQ ID NO: 17 - protein, 317 aa, Rattus norvegicus
MGQSNNVTEFVLLGFTQDPAGQKALFVMFSLMYIATMVGNLLIVGTVIVSPSLGSPMYFFLASLSLMDAVYSTAISPKLIVDLLREKKTISFRACISQLFIEHLFGGVDIVILVAMAYDRYVAICKPLHYLTIMNRRVCILFLVMAWAGGFAHALFQVLAVYNLPFCGPNIIDHFGCDIYPLLLLACTDTYFIGLSVIGNNGAMCIVIFSLLLFSYGIILRSLQNHSQEGRRKALSTCSSHITVVFLFFVPCIFMYVRPVSSFPIDKSITVIYTVVTPMLNPLIYTLRNSEMKNSMEKLLQKILSPNRLRLLCCLHY

SEQ ID NO: 18 - protein, 309 aa, Rattus norvegicus
METYNLTGTLEFLLLDLSEDTELQFIFFVLFLLIYLLTVLGNVLIILAISSDSQLHSPMYFFLYNLSLSDMGFSSTTVPKMLINMQTHNKSITYAGCLTQVFFFILFGGMDSLLLTAMAYDRWVAICHPLHYQVILNPRLCRCLLILSFFISVLSSQVHCLMVSQLTFCTNMEIHHFFCDVPELLKLSCSDSFISNIVRIILSIILGFLPMSGILYSYYKITSSIFKFPSLLGKYKVFSTCGSHLSVVCLFYGTGIGVYLSSTVSSSYGESMVASVLYIMVVPMINPFIYSLRNKDIKKALQKIVRQIL

SEQ ID NO: 19 - protein, 319 aa, Mus musculus
MDRSNETAPLSGFILLGLSAHPKLEKTFFVLILMMYLVILLGNGVLILVSILDSHLHTPMYFFLGNLSFLDICYTTSSVPLILDSFLTPRKTISFSGCAVQMFLSFAMGATECVLLSMMAFDRYVAICNPLRYPVVMNKAAYVPMAASSWAGGITNSVVQTSLAMRLPFCGDNVINHFTCEILAVLKLACADISINVISMVVANMIFLAVPVLFIFVSYVFILVTILRIPSAEGRKKAFSTCSAHLTVVLVFYGTILFMYGKPKSKDPLGADKQDLADKLISLFYGVVTPMLNPIIYSLRNKDVRAAVRNLVGQKHLTE

SEQ ID NO: 20 - protein, 318 aa, Mus musculus
MEGANQSTVAEFVLLGLSDHPKLEKTFFVLILLMYLVILLGNGVLILVSILDSHLHTPMYFFLGNLSFLDICYTTSSIPLVLDGFLTPRKTISFSGCAVQMFLSFAMGATECVLLGMMAFDRYVAICNPLRYPVVMNKSAYVPMAVSSWVAGGANSLVQISLAVQLPFCGDNVINHFTCEILAVLKLACADISINVISMGVANVIFLGVPVLFIFVSYIFILSTILRIPSAEGRKKAFSTCSAHLTVVLVFYGTILFMYGKPKSKDPLGADKQDVSDKLISLFYGVLTPMLNPIIYSLRNKDVKAAVRNLVGQKCLIQ

SEQ ID NO: 21 - protein, 318 aa, Mus musculus
MDVSNQTTVTEFVLLGLSAHPKLEKTFFVLILSMYLVILLGNGVLILVSILDSHLHTPMYFFLGNLSFLDICYTTSSVPLVLDGFLTPRKTISFSGCAVQMFLSFAMGATECVLLGMMAFDRYVAICNPLRYPVVMNKAAYVPMAVSSWVAGGANSLVQISLAVQLPFCGDNVINHFICEILAVLKLACADISINVISMGVANVIFLGVPVLFIFVSYIFILSTILRIPSAEGRKKAFSTCSAHLTVVIIFYGTILFMYGKPKSKDPLGADKQDLADKLISLFYGLLTPMLNPIIYSLRNKDVKAAVRNLASHRCLTF

SEQ ID NO: 22 - protein, 319 aa, Mus musculus
MERSNKTTPVSSFILLGLSAHPKLEKTFFVLILLMYLVILLGNVVLILVSILDSHLHTPMYFFLGNLSFLDICYTTSSVPLILDSFLTPRKTISFSGCAVQMFLSFAMGATECVLLGMMAFDRYVAICNPLRYPVVMSKAAYVPMAAGSWVSGSITATVQISLAMTLPFCGDNVINHFTCEILAVLKLACADISINVISMAVANAMFLGVPVLFIFVSYIFILSTILRIPSAEGRKKAFSTCSAHLTVVLVFYGTILFMYGKPKSKDPLGADKQDLADKLISLFYGVVTPMLNPIIYSLRNKDVKAAVTNLVGQKHFKW

SEQ ID NO: 23 - protein, 319 aa, Mus musculus
MVQGNWTSVTVFVFLGFSHYPRIEVTVFVLCLLMYLITLLGNTILISITILDSHLHTPMYFFLSNLSFLDIWYTSSALTPMLANFVSGKNTISFSGCASQMYFSLAMGSTECVLLSMMAYDRYVAICNPLRYPIIMNRRVCVQIAGSSWVTGCLTALVETGPVIHLSLCGNSIINHFTCEILALLKLACGDTSMVQLIMLVISILLLPLPMLLICVSYASILSNILRISSMDGRSKAFSTCAAHLTVVVLFYGTALSMYLKPSSVNSQEIDKFMALIYTGLTPMLNPIIYSLRNKEVKMAVKKLLMKNPFSAILTSVLK

SEQ ID NO: 24 - protein, 309 aa, Homo sapiens
MKINQTILKEFILVGFSVYPHVQTFLFVVFFCLYLLTLAGNLIIMGLTWVDRSLHTPMYLFLSALSFSETCYTLTIVPKMLEDLLAKDRSISVTGCSLQMCFFLGLGGTNCIILTLMGYDRFLAICNPLRYPLLMTNIVCGQLVASACTAGFFISLTETALIFRDSFCRPNLVKHFFCHMLAVIRLSCIDSNHTEFIITLISVSGLLGTLLLIILTDVFIISTVLRIPSAEGKQKAFTTCASHLTVVIIHFGFASIVYLKPEASGDDTLIAVPYTVITPFLSPIIFSLRNKDMKNAFRRMMGNTVALKK

SEQ ID NO: 25 - protein, 312 aa, Homo sapiens [sequence omitted]

SEQ ID NO: 26 - protein, 319 aa, Homo sapiens
MEKANETSPVMGFVLLRLSAHPELEKTFFVLILLMYLVILLGNGVLILVTILDSRLHTPMYFFLGNLSFLDICFTTSSVPLVLDSFLTPQETISFSACAVQMALSFAMAGTECLLLSMMAFDRYVAICNPLRYSVIMSKAAYMPMAASSWAIGGAASVVHTSLAIQLPFCGDNVINHFTCEILAVLKLACADISINVISMEVTNVIFLGVPVLFISFSYVFIITTILRIPSAEGRKKVFSTCSAHLTVVIVFYGTLFFMYGKPKSKDSMGADKEDLSDKLIPLFYGVVTPMLNPIIYSLRNKDVKAAVRRLLRPKGFTQ

SEQ ID NO: 27 - protein, 317 aa, Homo sapiens
MGEINQTLVSEFLLLGLSGYPKIEIVYFALILVMYLVILIGNGVLIIASIFDSHFHTPMYFFLGNLSFLDICYTSSSVPSTLVSLISKKRNISFSGCAVQMFFGFAMGSTECLLLGMMAFDRYVAICNPLRYPIILSKVAYVLMASVSWLSGGINSAVQTLLAMRLPFCGNNIINHFACEILAVLKLACADISLNIITMVISNMAFLVLPLMVIFFSYMFILYTILQMNSATGRRKAFSTCSAHLTVVIIFYGTIFFMYAKPKSQDLIGEEKLQALDKLISLFYGVVTPMLNPILYSLRNKDVKAAVKYLLNKKPIH

SEQ ID NO: 28 - protein, 317 aa, Homo sapiens; identical to SEQ ID NO: 27

SEQ ID NO: 29 - protein, 320 aa, Homo sapiens [sequence omitted]

SEQ ID NO: 30 - protein, 318 aa, Homo sapiens
MEWENHTILVEFFLKGLSGHPRLELLFFVLIFIMYVVILLGNGTLILISILDPHLHTPMYFFLGNLSFLDICYTTTSIPSTLVSFLSERKTISLSGCAVQMFLSLAMGTTECVLLGVMAFDRYVAICNPLRYPIIMSKDAYVPMAAGSWIIGAVNSAVQTVFVVQLPFCRNNIINHFTCEILAVMKLACADISGNEFILLVTTTLFLLTPLLLIIVSYTLIILSIFKISSSEGRSKPSSTCSARLTVVITFCGTIFLMYMKPKSQETLNSDDLDATDKLIFIFYRVMTPMMNPLIYSLRNKDVKEAVKHLLRRKNFNK

SEQ ID NO: 31 - protein, 318 aa, Homo sapiens
MEWENHTILVEFFLKGLSGHPRLELLFFVLIFIMYVVILLGNGTLILISILDPHLHTPMYFFLGNLSFLDICYTTTSIPSTLVSFLSERKTISLSGCAVQMFLGLAMGTTECVLLGMMAFDRYVAICNPLRYPIIMSKDAYVPMAAGSWIIGAVNSAVQSVFVVQLPFCRNNIINHFTCEILAVMKLACADISDNEFIMLVATTLFILTPLLLIIVSYTLIIVSIFKISSSEGRSKASSTCSAHLTVVIIFYGTILFMYMKPKSKETLNSDDLDATDKIISMFYGVMTPMMNPLIYSLRNKDVKEAVKHLLNRRFFSK

SEQ ID NO: 32 - protein, 318 aa, Homo sapiens
MEWENQTILVEFFLKGHSVHPRLELLFFVLIFIMYVVILLGNGTLILISILDPHLHTPMYFFLGNLSFLDICYTTTSIPSTLVSFLSERKTISFSGCAVQMFLGLAMGTTECVLLGMMAFDRYVAICNPLRYPIIMSKNAYVPMAVGSWFAGIVNSAVQTTFVVQLPFCRKNVINHFSCEILAVMKLACADISGNEFLMLVATILFTLMPLLLIVISYSLIISSILKIHSSEGRSKAFSTCSAHLTVVIIFYGTILFMYMKPKSKETLNSDDLDATDKIISMFYGVMTPMMNPLIYSLRNKDVKEAVKHLPNRRFFSK

SEQ ID NO: 33 - protein, 314 aa, Homo sapiens [sequence omitted]

SEQ ID NO: 34 - protein, 316 aa, Homo sapiens
MQGENFTIWSIFFLEGFSQYPGLEVVLFVFSLVMYLTTLLGNSTLILITILDSRLKTPMYLFLGNLSFMDICYTSASVPTLLVNLLSSQKTIIFSGCAVQMYLSLAMGSTECVLLAVMAYDRYVAICNPLRYSIIMNRCVCARMATVSWVTGCLTALLETSFALQIPLCGNLIDHFTCEILAVLKLACTSSLLMNTIMLVVSILLLPIPMLLVCISYIFILSTILRITSAEGRNKAFSTCGAHLTVVILYYGAALSMYLKPSSSNAQKIDKIISLLYGVLTPMLNPIIYSLRNKEVKDAMKKLLGKITLHQTHEHL

SEQ ID NO: 35 - protein, 314 aa, Homo sapiens
MLMNYSSATEFYLLGFPGSEELHHILFAIFFFFYLVTLMGNTVIIMIVCVDKRLQSPMYFFLGHLSALEILVTTIIVPVMLWGLLLPGMQTIYLSACVVQLFLYLAVGTTEFALLGAMAVDRYVAVCNPLRYNIIMNRHTCNFVVLVSWVFGFLFQIWPVYVMFQLTYCKSNVVNNFFCDRGQLLKLSCNNTLFTEFILFLMAVFVLFGSLIPTIVSNAYIISTILKIPSSSGRRKSFSTCASHFTCVVIGYGSCLFLYVKPKQTQAADYNWVVSLMVSVVTPFLNPFIFTLRNDKVIEALRDGVKRCCQLFRN

SEQ ID NO: 36 - protein, 310 aa, Homo sapiens
MMDNHSSATEFHLLGFPGSQGLHHILFAIFFFFYLVTLMGNTVIIVIVCVDKRLQSPMYFFLSHLSTLEILVTTIIVPMMLWGLLFLGCRQYLSLHVSLNFSCGTMEFALLGVMAVDRYVAVCNPLRYNIIMNSSTCIWVVIVSWVFGFLSEIWPIYATFQFTFRKSNSLDHFYCDRGQLLKLSCDNTLLTEFILFLMAVFILIGSLIPTIVSYTYIISTILKIPSASGRRKAFSTFASHFTCVVIGYGSCLFLYVKPKQTQGVEYNKIVSLLVSVLTPFLNPFIFTLRNDKVKEALRDGMKRCCQLLKD

SEQ ID NO: 37 - protein, 312 aa, Homo sapiens
MRNGTVITEFILLGFPVIQGLQTPLFIAIFLTYILTLAGNGLIIATVWAEPRLQIPMYFFLCNLSFLEIWYTTTVIPKLLGTFVVARTVICMSCCLLQAFFHFFVGTTEFLILTIMSFDRYLTICNPLHHPTIMTSKLCLQLALSSWVVGFTIVFCQTMLLIQLPFCGNNVISHFYCDVGPSLKAACIDTSILELLGVIATILVIPGSLLFNMISYIYILSAILRIPSATGHQKTFSTCASHLTVVSLLYGAVLFMYLRPTAHSSFKINKVVSVLNTILTPLLNPFIYTIRNKEVKGALRKAMTCPKTGHAK

SEQ ID NO: 38 - protein, 313 aa, Homo sapiens
MGDRGTSNHSEMTDFILAGFRVRPELHILLFLLFLFVYAMILLGNVGMMTIIMTDPRLNTPMYFFLGNLSFIDLFYSSVIEPKAMINFWSENKSISFAGCVAQLFLFALLIVTEGFLLAAMAYDRFIAICNPLLYSVQMSTRLCTQLVAGSYFCGCISSVIQTSMTFTLSFCASRAVDHFYCDSRPLQRLSCSDLFIHRMISFSLSCIIILPTIIVIIVSYMYIVSTVLKIHSTEGHKKAFSTCSSHLGVVSVLYGAVFFMYLTPDRFPELSKVASLCYSLVTPMLNPLIYSLRNKDVQEALKKFLEKKNIIL

SEQ ID NO: 39 - protein, 319 aa, Homo sapiens
MFPANWTSVKVFFFLGFSHYPKVQVIIFAVCLLMYLITLLGNIFLISITILDSHLHTPMYLFLSNLSFLDIWYSSSALSPMLANFVSGRNTISFSGCATQVYLSLAMGSTECVLLPMMAYDRYVAICNPLRYPIIMNRRTCVQIAAGSWMTGCLTAMVEMMSVLPLSLCGNSIINHFTCEILAILKLVCVDTSLVQLIMLVISVLLLPMPMLLICISYAFILASILRISSVEGRSKAFSTCTAHLMVVVLFYGMALSMHLKPSAVDSQEIDKFMALVYAGQTPMLNPIIYSLRNKEVKVALKKLLIRNHFNTAFISILK

SEQ ID NO: 40 - protein, 319 aa, Rattus norvegicus
MVQGNWSSVTVFFFLGFSQYPRIEVTIFVLCLLMYLITLLGNTILISITILDSHLHTPMYFFLSNLSFLDIWYTSSALTPMLANFVSGENTISFSGCAFQMYFSLAMGSTECVLLSMMAYDRYVAICNPLRYPIIMNRRVCVQIAGSSWVTGCLTALVETGPVIHLSLCGNNIINHFTCEILALLKMACGDTAMVQLIMLVISILLLPLPMLLICVSYASILSNILRISSVDGRSKAFSTCAAHLTVVVLFYETALSMYLKPLSVNSQEIDKFMALIYVGLTPMLNPIIYSLRNKEVKMAVKKLLIKNPFTAILTSVLK

SEQ ID NO: 41 - DNA, 960 bp, Rattus norvegicus
atggacagatccaacgagacttcccttgtgtcctacttcattctcctcggcctctctgcccacccaaggctggagaaaaccttcttcgtgctcatcctgctgatgtacctggtgatcctgctgggcaatggggtccttatcctggtgaccatcctcgactcccacttgcacacgcccatgtacttcttcctggggaacctctccttcctggacatctgctacaccacttcctcaatccccttggtcctggatggttttctcacccccaggaagaccatctccttctcaggctgtgccatgcagatgtttctgtccttcgccatgggagccacagagtgtgtgctcctgggcatgatggcgtttgatcgctatgtggccatctgcaacccccttaggtacccagtggtcatgagcaaggctgcctatgtccccatggccgccggctcctgggcagggggaagcatcactgccacagttcagacatctttagcaatgcggctgccattctgtggggacaacgtcatcaaccacttcacctgtgagatcctggctgtcctgaaactggcctgtgccgacatctccatcaatgtcatcagcatggcggtaggtaatgcagtatttttgggggtcccagtcctgttcatctttgtctcctacatcttcatcctctccaccatcctgaggatcccctctgctgaggggaggaagaaggccttctccacctgctctgcccacctcactgtggtgattgtcttctatgggaccatcctcttcatgtacgggaagcccaaatccaaggacccactgggggcagacaagcaggaccttgcagacaagctcatctccctcttctatggggtgttgacccccatgttgaaccccatcatctacagcctgaggaacaaggacgtgagggccgctgtgaggaaccttctacatcagaaacacttcaagtggtga

SEQ ID NO: 42 - DNA, 948 bp, Rattus norvegicus
atggaaagtagcaaccagacctcccccgtgtcccgcttcattctcctgggcctctctgcccacccaaccctggagaaaaccttcttcgtgctcatcctgctgatgtacctggtgatcctgctgggcaacggggtcctcatcctggtgaccatcctcgactcccacctgcacacgcccatgtacttcttcctggggaacctctccttcctggacatctgctacaccacctcctcggtccccctcattctggacagcttcctgacccccaggaagaccatctccttctcaggctgtgccgtgcagatgtttctgtctttcgccatgggagccacggagtgtgtgctcctgagcatgatggcgtttgatcgctatgtggccatctgcaatcccctcagatacccagtggtcatgagcaaggctgtctatgtccccatggccaccggctcctgggcagctggaattgcagcctctctggttcaaacatcccttgcaatgcggctgcccttctgtggggacaacgtcatcaatcactttacctgtgagatcctggctgtcctgaaactggcctgtgctgacatctccatcaatatcatcagcatgggggtgacaaatgtaatcttcctgggggtcccagttctctttatttctttctcctacatattcatcctctccaccatcctgaggatcccctctgctgaggggaggaagaaggcattctccacctgctctgcccacctcactgtggtgattgtcttctatgggaccatcctcttcatgtacgggaagcccaagtccaaggacccactgggggcagacaagcaggaccttgcagacaagctcatctccctcttctatggggtgttgacccccatgttgaaccccatcatctacagcctgaggaacaaggacgtgagggccgctgtgaggaacctggtggtccttaagtaa

SEQ ID NO: 43 - DNA, 957 bp, Rattus norvegicus
atggaaatagccaaccagtccacggtagctgagtttgtcttgctgggattgtcagaccacccaatgctggagaaaaccttcttcgtgctcatcctgctgatgtacctggtgatcctgctgggcaatggggtccttatcctggtgaccatcctcgactcccacctgcacacgcccatgtacttcttcctggggaacctctccttcctggacatctgctacaccacttcctcaatccccttggtcctggatggttttctcactcccaggaaaaccatctccttctcaggctgtgccatgcagatgtttctgtctttcgccatgggagccacggagtgcgtgctcctgggcatgatggcgtttgatcgctatgtggccatctgcaacccccttaggtaccccgtggtcatgagcaaggctgcctatgtgcccatggccgtcagctcctgggtggctggcggtgccaactccatggtgcagatctcccttgctgtgcagttgcctttctgtggggacaatatcatcaaccacttcacctgtgagattttggcagtcttaaagctagcctgtgctgacatctccatcaatgtcatcagcatgggggtggccaacgtgatcttcctgggggtcccagttctgttcatctttgtctcctatatattcatcctctccaccatcctgaggatcccctctgctgaggggaggaagaaggcattctccacctgctctgcccacctcaccgtggtgattgtcttctatgggaccatcctcttcatgtacgggaagcccaagtccaaggacccactgggggcagacaagcaggaccttgcagacaagctcatctccctcttctatggggtgttgacccccatgttgaaccccatcatctacagtctgaggaacaaggacgtgaagactgctgtaagagacctggtgggtcgaaaatgcctaattcagtga

SEQ ID NO: 44 - DNA, 957 bp, Rattus norvegicus
atggaagtgtccaaccagtccactgtgacagaatttgtcctattgggcctctctgcccacccaaccctggagaaaaccttcttcgtgctcatcctgctgatgtacctggtgatcctgctgggcaatggggtcctcatcctggtgaccatccttgactctcacctgcacacgcccatgtacttcttcctggggaacctctccttcctggacatctgctacaccacctcctcggttcccttggtcctggatggttttctcacccccaggaagaccatctccttctcaggctgtgccgtgcagatgtttctgtctttcgccatgggagccacggagtgcgtgctcctgggcatgatggcgtttgatcgctatgtggccatctgcaacccccttaggtaccccgtggtcatgagcaaggctgcctatgtgcccatggccgtcagctcctgggtggctggcggtgccaactccatggtgcagatctcccttgctgtgcagttgcctttctgtggggacaatgtcatcaatcatttcatttgtgagattttggcagtcttaaagctagcctgtgctgacatctccatcaatgtcatcagcatgggggtggccaacgtgatcttcctgggcgtcccagttctgttcatctttgtctcctacatcttcatactctccaccatcctgaggatcccctctgctgaggggaggaagaaggcattctccacctgctctgcccacctcactgtggtaatcattttttatggtaccatcctcttcatgtacgggaagcccaagtccaaggacccactgggggcagacaagcaggaccttgcagacaagctcatctccctcttctatggacttctgactcccatgttgaaccccatcatctacagcctgaggaacaaggatgtgaagactgctgtaagagacctggcaagtcacagatgtctcacccagtaa

SEQ ID NO: 45 - DNA, 960 bp, Rattus norvegicus
atggacaggttcaatgagacctcccccgtatcccacttcattctcctgggcctctccgcccacccaaggctggagaaaaccttcttcgtgctcatcctgctgatgtacctggtgatcctgctgggcaacggggtccttatcctggtgaccatcctcgactcccacctgcacacgcccatgtacttcttcctggggaacctctccttcctggacatctgctacaccacctcctcggtccccctcattctggacagcttcctgacccccaggaagaccatctccctctcaggctgtgccgtgcagatgtttctgtccttcgccatgggagccacggagtgtgtgcttctgagcatgatggcgtttgatcgctatgtggccatctgcaacccccttaggtacccagtggtcatgagcaaggctgcctatgtgcccatggccgccggctcctgggcaggtggtatcaccaattctgtagttcagacatctttggcaatgaggctgccgttctgtggggacaatgtcatcaaccacttcacctgtgagatcctggctgtcctgaaactggcctgtgctgacatctccatcaatatcatcagcatgggggtggccaacgtgatcttcctgggggtcccagttctgttcatctttgtctcctacatctttatcctctccaccatcctgaggatcccctctgctgaggggaggaagaaggcattctccacctgctctgcccacctcaccgtggtgattgtcttctatgggaccatcctcttcatgtacgggaagcccaagtccaaggacccactgggggcagacaagcaggaccttgcagacaagctcatctccctcttctatggggtggtgacccccatgttgaaccccatcatctacagcctgaggaacaaggatgtgagggctgctgtgaggaacctggtggttcagaaacacctaactgagtga

SEQ ID NO: 46 - DNA, 960 bp, Rattus norvegicus
atggtacagggaaattggtcctctgtcacagtgtttttcttcctgggattttctcagtaccccagaattgaagtcaccatatttgtgctgtgtctgctgatgtacctgatcaccttgctgggaaatactattctgatctccatcactatccttgattctcacctgcacactcccatgtactttttccttagcaacctctcctttctggacatctggtatacctcttctgctctcactcctatgctggcaaactttgtttcaggggaaaacaccatctcattctcaggatgtgctttacagatgtacttctctcttgccatgggctccacggaatgtgtgctcctctctatgatggcctatgacaggtatgtggccatctgcaaccccctgagatatcccatcattatgaacaggagggtctgtgtacagattgcaagcagctcctgggctacaggctgcctcactgccttggtggaaactggacctgtgattcatctgtctctctgtggtaataacatcatcaatcatttcacctgtgaaattctggctctcttaaaaatggcttgtggagacactgccatggtgcagttaattatgttagtgatcagcatccttcttctcccattgccaatgttgctcatttgtgtctcctatgcatccatcctctccaacatcttgaggatcagctcagtggatggccgaagcaaagccttttcaacatgtgcagcccacttgactgtggtggttctgttctatgggacagctctctccatgtacctgaagccctcatctgtaaactcacaggaaatagataaatttatggcattgatatatgctggactaacaccaatgctaaatcctattatctacagtctacggaacaaagaagtcaaaatggctgtgaaaaaattgttgattagaaatcccttcagtactatcttaacttctgtcctcaaataa

SEQ ID NO: 47 - DNA, 924 bp, Rattus norvegicus
atggcaccaataaaccagtcagttgtgaccatgttcttcctgcaaaactttgttgatgatccctggatccagaatgttctcttttgctttttctttgccttgttcgtggcagccatagctggcaatggcctgattatcacagtcattcacagcagtgccaacctccacactcccatgtacttcttcctagtcaatctttccctcatggatgtgatttgcactgtgacagtgttgcccaaagtcctgcagagcctggtggcagagaacgccatttcttatgggggatgtctcacacagatgtttgtcttctcctgggttctgggctctgagcttctgcttttctctgccatggcctacgaccgctaccttgcaatctgccggccattgcactatggtaccctcatgagtggcagggtctgcatagcccttgcaacctttgtgtggttcactggagctctcaattccttggtgctcacttgtctggtgttgccactgtccttctgtggtcccaatctcatcacacacttcttctgtgagatcccttctgtgttgatgctgtcctgcagccccacctttatcaatgacatcatgactgtcattgcagacatgttccttacaggactgaacttcctattgactatgacatcctatggctttatcattgccagcatcctgcgcatccgctctgctgagggcaagaagcgtgccttctctacctgctctgcccacctggttgtggtcaccctttattattctactgttctatatacttatgtccggccagccctaggaacttctgggctcctggacaaagtcattgctgttctgtataccactgtgaccccatctctgaacccactcatctataccctgagaaacaaggaattcaaaacatcctttaaaaaactcttatttcccaattga

SEQ ID NO: 48 - DNA, 936 bp, Rattus norvegicus
atggacagtttcaatgccactttagaagagagattcattttggtgggcttttcagattggcctcagctggaactcatcctttttgtttttatttcaattgtctactccctaactctctttggcaacaccgccatcatagctctctcccgaatggaccttcgattacacacccccatgtacttcttcctctcacacctctccttcctggacctctgctacaccaccagcaccgtgccccagctcctgatcaacctgcatggacttgacaggatcatcagctatggaaggtgtgtggcccagctgtttatatctcttgctctgggatccactgagtgtgtgctcctggtggtgatggcttttgaccgctatgctgctgtgtgtcgtccactgcactacacgaccatcatgcaccctgttctctgccaggcattggccattgcttcctgggtaggaggcttcttgaactctctgattcaaacaggactcatgatgaccataccgctctgtggacgccgactaaatcatttcttctgtgagatgcctgtgttcctcaagttagcctgcaaggacacaggagatacagaggccaagatgtttgtggccagagccataatcttggtcttccctgcaacattaattcttggctcctatggacacattgctagagcagtattgaaggtaaagtcaatggctggccgcagaaaggcttttgggacatgtgggtcccacctcctggtggtttccctgttttatggctcagccatttacacatacttgcaacccaagggtagttattctgagagtgatggaaagtttgttgcccttttttatactatagtgactcccatgctcaaccctttgatctataccctgaggaacaaggatgtgaagggcgctctgtggaaggtgctagggaggagcacaggccaagagtag

SEQ ID NO: 49 - DNA, 918 bp, Rattus norvegicus
atgatgaatgggacactggtcactgagttcttcatcctgggattctcagaaatgcctcaccttcgggtaccacttctcctcagcttcttctgcctatacatggctgcaatctcaggaaacctgcttattatggtgacaatcagtgccagcccagccctgcatacccctatgtacttcttcctggtcaacttggccatggtggatatcctctgcacctccaccatcctaccaaagctactggacagcatgatggcagggaggagcatctcttatgggggctgcatggcccagctcttcttcttcacatggtccctgggggcagagcttctgctcttctcagctatggcctatgaccgcttcgtggccatctgctgtcccctgcactacagtgcttggatgggccctagggtatgtgcattcctggctggccttgtctggtccatcagcataactaacaccagcgtgcacacaggactgatgttgcgtctaccattctgtggctccaatgagatagagcacttcttctgtgagattcccccactgttgaaactctcctgtgctccaacacaattgaatgaggccatggcctttgctgcagatgtgttcctggctgtagggaacttctctgtgacaatcctctcctatggctttattgtggttagtatcctgaggatccgttcagctgaaggcaagcgacgtgctttttctacctgctctgcacacctcattgtggtcaccatgtactactccactgtcatctatacctacattcgccctgcatccagctactcactgaacaaggataaggtggtgtccatcatctacacctcggtggcacccaccttgaaccccctcatctacactctgagaaacaaggatgtcaaagttgcactccggagacttctatcctgctcctga

SEQ ID NO: 50 - DNA, 954 bp, Rattus norvegicus
atggacagccccagcaatagcaccatatcccgtggctttctccttcaaggcttttttgagtttccgcatctgaggccccttctcttcctggtgctgctggctgtgcacctggccactttgagtggtaacctgctcatcctggtggctgtggtctcagtgcccagcaggccacccatgctgctcttcctttgtcagttgtcagccattgaactctgttacacgctagtggtggtgccccgatctctagccgacctgagcatgcctggccatggcaggggcagccccatctccttcctgggctgtgctgtccagatgcagatgtttgtggcactgggtggggctgagtgcttcttgttggcagccatggcctatgaccgctatgtggccatctgccacccactgcgctatgcatccattgtgaccccaggtctatgtgcacgactggccctggcctgctgccttggcggactagctgtatccgtgggactaacggtggcagtgttccacctccctttctgtggctcccgcctgctggtgcatttcttctgcgacatcaccgccctgctgcacttggcctgcacacggagctacatagatgagctgcccttgctaggagcctgcctggtgctgctgttgctgccctcaatgctcatcctgacctcctatggggccattgcctctgccctgcgccgcctgcatggtggtggaggcagacgcaaggctgcctcgacctgtgcttcacacctggctgtcaccttcttgcattacggctgtgccaccttcatgtacgtgcggcccaagtccagttactccccgcgactggatcgcacactagcgctggtttataccaacgtcacaccacttctatatcctctcatctacagcctgcgcaaccgggagatcacttctgccattcacagagtcctggggctccgtggatcagggtctatgtga

SEQ ID NO: 51 - DNA, 927 bp, Rattus norvegicus
atgggatatgggaacatcacagaatttatcttactggggcttttccatgatgaggatgtcaaggcaatatgtgctgtgttattcttactatgctatcttgcaattctctgtggaaacttgattgttcttctcactatcaagggcagtcagcttagtgagcagccgatgtacttttttctcagctatctgtctttcatggatgtctgcttcacttccacagtagccccaaaatttattataggactattggtacaatgtaacacaatctcctacaatgcctgcatagcacaaatgttttatgcccacttctttggtgccactgagatatttatattggtggtcatggcctatgatcgctatgtagcaatttgcagacctctttactacatgatcacgatgagcagacaggtatgctacattcttgtgataggctcagtttttggtgcttttatacactcacttgtgcatgtattagttattatcagacttcctttttgtggcaccaatgaaatagaccactacttctgtgacatattccctctgctgaagctggcctgcactgagacaagactccttgttattgtaatcattaccactacaggggtgatgtccattttgacttttgttgcattggtaatttcttacatcatcattttgtccatcttgaggaccaggtcatcggaaggccgtcgcaaagctctttctacctgtggctcacatatcaccgtagtgttcatgttcttcttgccactcatctttacctatgttccaatgggtgattctgttggggatgacaaggtgtttgctctgttttacaccatgattgcaccattgttcaaccctctcatctacacactgaggaatacagacatgaagaatgccatgaggaaggtgtggtgccaagacaaactgtttgaaggaaagtga

SEQ ID NO: 52 - DNA, 939 bp, Rattus norvegicus
atgaatgaaataaattacaccaaggtttctgaatttgtgtttctgggactttcaaaatctaaagaaatacaacatttcttccttgccttctctgtggtattatatgtagccatcgtactgggcaatacccttgttgtgtttacactagcattggacccacatttacattcccctatgtattttcttttaggaaacctctcatttattgatttatgtttttccaccttaactgtacccaagatgatttctgacttgtcgtctgggcacaataccatctcattccatggttgtgtcttccagatatttgtcattcatgtccttggggcttctgagatggtattgctggtagtcatggcctgggacagatatgtagccatatgtaaacccctccactatttaaccatcatgaacccacggatttgcattttgcttctatctggggcttggattattgcatttttgcattcagtgatccaacttggttttgttgtccatttgaggttttgtggtccgaatgagatagatagtttttactgtgatcttcctagattcatcaagctggcctgcatggacaactacagaatggagttcatggtggctgccaacagtggcatcatttctattggcaccttcttcttattgattatctcctacattgttatcctgctcactgtgaggaaacattcatcaggagatttgtccaaggccctctcgacactttctgctcacatctctgtggtagttttgttctttggaccatgcatctttgtatacatgtggccatttcctacagtgccagtggataagttccttgccattctggacttcatgattacacccatcctgaaccctgccatttacacactgaggaacaaagacatgaaggtggcaatgaggaaactaagtgttcagctcctgcattttaggaaaatgtcctga

SEQ ID NO: 53 - DNA, 927 bp, Rattus norvegicus
atgagcctctcagacaatccagaactgcagcctatcctttttgtactgtttttatcattctacatggtcacagtgcttggaaatctgctcattattctggctgtgagctctgacttccatctccatacccccatgtatttctttctctccaacctttcctggtctgacatttgtttgatctccactacagttccaaagatgatttgggatattagaactcaaagcagagtcatctcctatgcgggctgcctaacacagatgtccatgttcataatttttggatgtatggatagcatgcttctgactgtaatggcctatgacaggtttgtggccatttgtcaccccctgaattacaatatcattatgaatcctaacctctgtgccctcttacttttggcatctgtattggccagtcttgtggattcgcaggtgcacaatttgattgtgctacaatttacatacttcaatgacatggaaatctctaactttttttgtgagccttcacaactgcttaatctcaagtgttctgaaatgttcaccaaaaacatagttatacgattcattggtgtcttttttggtttattttcaacctcaggaattattttctcatactataaaattgtgtcttccatcctgagaattccatcaacagaaggaaagtataaagccttttctacctgtgtgtctcacctgtcagttgtgtgcttattttatggaacagccattggagtatacattggttcaactgcatcaaactcttctgaaaactgtaaagttgcttcactgatgtacacagtggtcacacctatgcttaatcccttcatctacagcctgaggaacagggacattaaaactgccctatggaaactacagaaaaaagcaaaattgcacaatgccttccatcattttttaattttgagttcttaa

SEQ ID NO: 54 - DNA, 942 bp, Rattus norvegicus
atgagcagcaccaaccattccagtgtctcagtgttcctcctcctgggactctccaggcagccccagcagcagcagctcctcttcctgctcttcctcatcatgtacctggccactgtcctgggaaacctgctcatcatcctgtccatcagcacagactcccgcctgcacacccccatgtacttcttcctcagcaacctgtcctttgtggatctctgcttctcctccaccactgtccctaaagtactgaccaaccatatacttgggagtcaggaaatttccttctctgggtgtctcacacagatgtattttctctctgtgtttgctgatatggacaattttctgctggctgtaatggcctatgaccgatttgtggccatatgccaccctttacactacacaaaaaagatgacccatcagctgtgtgcccttcttgttgttgagtcatggctggctgtcaatctgaatgctcttttacatacactgctaatggctcgactttctttctgtggggacaacctcataccccactatttctgtgatgcaactcctctcctgaaactctcctgctcagacacacatctcaatgagctaatgattaatacagagggagctgtgaccatggtcaccccatttgtctgcatcctgatctcctacatccacatcacctgtgctgtcctgagagtctcatcccccaggggaggatggaaagccttctccacctgtggctcccacctggctgtggtctgcctcttctatggcaccatcatcgctgtgtatttcaatccctcaactgcccattcacctgagaaggacacagtagccactgtgttgtacacagtggtgactcccatgctgaacccctttatctatagcctgaggaacagggacctgaaatggtcactacgaaaacttgtccatagaagtaccttttcttttcagtga

SEQ ID NO: 55 - DNA, 948 bp, Rattus norvegicus
atgatgaaggcaaatcactctttagagttcattctcataggattcacagatcacccagacctaaagacacttctgttcctggtgttctctgccatatatctggtcaccatggtggggaatcttgggctggtggccttgatctacatggagcctcgtttccacacacccatgtacatctttctgggaaacctggccctgatggactcctgctgctcctgtgccatcactcccaagatgctagagaacttcttttctgtggacagaaggatctctttctgtgaatgcatggcacagttctatttcctctgtcttgctgaaactgcagactgctttcttctggcagccatggcctatgaccgctatgtggccatatgcaacccactgcagtaccacaccatgatgtccaagaagctctcccttcagatgagcatgggcatattcatagccagtaacctgcattccttgattcatacaggctgtctgttaagattaaatttctgtaaatctaccaaaattgatcacttcttctgtgatattcttcccctgtatcgcctctcctgtacaaacccttttattaatgaactaatgatatatattttttcaatgccaattcaagtctttaccattaccactatcttggtctcttacttctgcattcttttcactattttcaagatgaaatccaaggatgggagaggaaaagcattttctacttgtgcatcccattttttttctgtgtcaatattctacacctgtcttctcatgtatattggaccatctgaagatggtagtaaagacataccagtagctgtgttttatacagtaataattcctttgttaaacccttttatttacagcctgagaaataaggaggtagtaaatgctgttaagaaagttatgaaaacttacagtatttttaaaaatgcttctgcttccatagtacattaa

SEQ ID NO: 56 - DNA, 951 bp, Rattus norvegicus
atggtaccaatggaaagaaatgtgagtgtggagattatctttgtcctggtgggcttcacagactaccctgagcttcagatccccctcttcctagtctttctcttcatgtacatcatcactgtagtggggaaccttgggatgatagtgctcatcaatatcgaccccaagttccacactcccatgtactttttccttagccacctttcctttgttgatttctgttatccaacaattatcatgcccaagctgcttgagaacttgatcctggctgataaaactatactttatttcagctgcatgctgcagtacttcctgtcctgtgtggctgtggtggctgagtcctacttgttagcagttatggcctatgaccgctttgtggccatctgtaatcccttgctgtatactgtggccatgtcacagagactgtgcatcctgcttgtgactggatcatatatctggagcacattggccaccttgattctcctctgctatgctctgcagctaaagttctcaagatttactgtgatcaaccacttcttctgtgaatatactgccctcattgctgtctctagttctgatatacacatccccagcctactactcttttgttttgcaacctttaacgaagtgagtaccctactgatcattctcacttcttacgtgttcatttttgtgactgtgttaaaaatcaaatctgctagtggacgtcgcaaagccttctctacctgtgcctcccacctgactgccatcaccatttttcatggaaccatactttctctgtactgtgtacctaacccacaaaattctaggaatgcagtcaaagtggcctctgtcttttacgcagttgtcaaccccttgcttaaccctcttatctacagtctgagaaacaaggatgtgaaagaggttttacagaaagtagtgagcacttcattgaaattcccacttcactga

SEQ ID NO: 57 - DNA, 954 bp, Rattus norvegicus
atgggacagagcaacaatgtcacagaatttgtcctgctgggcttcactcaggatcctgctgggcaaaaagctttatttgttatgttttcactgatgtacattgcaacaatggtgggcaacctgctcattgtagggacagtgattgtcagcccctccttaggctccccgatgtacttcttccttgcctctctgtcactcatggatgctgtttattctactgccatctcacccaagttgattgtagacttgctccgtgagaaaaagaccatctccttcagagcttgcataagccagctctttatagagcatttatttggcggggttgacattgttattctggtggcaatggcctatgatcgctatgtggccatctgtaagccactacattatttgaccatcatgaatcgacgggtatgtattctcttcttggtgatggcctgggcaggaggttttgcacatgctttgtttcaagttcttgctgtatataatcttcctttctgtggtcccaatatcattgaccactttgggtgtgacatatacccgctcttgctacttgcatgtactgacacctacttcattggactttctgtcattggcaataatggggccatgtgtatagtcatctttagtctccttctattttcctatggaataattcttagatctctccagaatcacagtcaggaagggaggcgcaaagctctgtccacctgcagctctcatatcactgtggtttttctcttttttgttccctgtattttcatgtatgttagacctgtttctagttttcctattgacaaatccatcactgtaatttatactgttgtcacccccatgttgaatcctttaatatatactttgagaaattcagagatgaaaaattctatggaaaagctcctgcagaaaatattaagtccaaatagactaaggcttctttgctgcttacattattaa

SEQ ID NO: 58 - DNA, 930 bp, Rattus norvegicus
atggaaacatataatttaacaggaaccttggaatttcttctgcttgatctgtcagaggacactgaactgcagttcatcttctttgtattgttcctgctcatatacctgctcactgtacttggaaatgtgctcatcatcttggccattagctctgattctcagctccatagtcctatgtactttttcctctacaacctttcattgtctgacatgggcttcagcagcaccacagtccccaaaatgctgataaatatgcagactcacaacaaatccataacttatgcaggctgcttaactcaggtattcttcttcattctttttgggggtatggacagcctattactgactgcaatggcctatgaccgatgggtagccatttgtcaccctctacactaccaagtcattctcaaccctcgtctgtgtagatgtttgctcatactttcatttttcattagtgttttgagttcacaggtacactgcttgatggtgtcacaattaacattttgcactaatatggaaatccatcatttcttctgtgatgttccagaacttctaaaactttcctgttctgactcctttatcagtaacatagttaggattattttaagcatcattcttggtttcctacccatgtcaggaatactttattcctactataaaataacttcctccatttttaaatttccatcactgttagggaaatataaagtcttctctacttgtggatctcacctatcagttgtttgcttattttatggaacaggtataggtgtgtaccttagttcaacagtttctagttcttacggggaaagcatggtggcttcggtactatatatcatggtggttcccatgataaaccccttcatctacagcctgaggaacaaggacatcaaaaaagcccttcagaaaattgtcagacaaatattgtag

SEQ ID NO: 59 - DNA, 960 bp, Mus musculus
atggacagatccaatgagaccgcccccctgtccggcttcattctcctgggcctctctgcccacccaaagctggagaaaaccttcttcgtgctcatcctgatgatgtacctggtgatcctgctgggcaacggcgtcctcatcctggtgagcatcctcgactcccacctgcacacgcccatgtacttcttcctggggaacctctccttcctggacatctgctacactacctcctctgtccccctcattctggacagctttctgactcccaggaagaccatctccttctcgggctgtgccgtgcagatgtttctctccttcgccatgggagccacggagtgtgtgctcctgagtatgatggcgtttgatcgttatgtggccatctgcaacccccttagatatcctgtggtcatgaacaaggctgcctatgtgcccatggctgccagttcctgggcaggtggtatcactaattctgtagtgcagacatctttggcaatgcggctgcccttctgtggggacaatgtcatcaatcacttcacctgtgagatcctggcagtcctgaaactggcctgtgctgacatctccatcaatgtcatcagcatggttgtggccaacatgatcttcttggcagtcccagtcctcttcatctttgtctcctatgtcttcatccttgtgacaatcctgaggatcccctctgctgaggggaggaagaaggcattctccacctgctctgcccacctcaccgtggtacttgtcttctatggaaccatcctcttcatgtacgggaagcccaagtccaaggacccactgggggcagacaagcaggaccttgcagacaagctcatctccctcttctatggagtggtgacccccatgctaaaccccatcatctacagcttgagaaacaaggacgtgagggctgctgtgaggaacctggtgggccagaaacacctaactgagtga

SEQ ID NO: 60 - DNA, 957 bp, Mus musculus
atggaaggagccaatcagtctacggtagctgaatttgtcttgctggggttgtcagatcacccaaagctggagaaaaccttcttcgtgctcatcctgctgatgtacctggtgatcctgctgggcaacggcgtcctcatcctggtgagcatcctcgactcccacctgcacacgcccatgtacttcttcctggggaacctctccttcctggacatctgctatactacctcctcgattcccttagtcctggatggttttctcacccccaggaagaccatctccttctcgggctgtgccgtgcagatgtttctctcctttgccatgggagccacggagtgtgtgctcctgggcatgatggcgtttgatcgttatgtggccatctgcaaccctcttagataccctgtggtcatgaacaagtctgcctatgtgcccatggccgtcagctcctgggtggctggcggtgctaactccttggtgcagatctcccttgcggtgcagttgcctttctgtggggacaatgtcatcaatcacttcacctgtgagatcctagcagtcttaaagctagcctgtgctgacatctccatcaatgtgatcagcatgggggtggccaatgtgattttcctgggggttccagttctgttcatctttgtctcctacatcttcatactctccaccatcctgaggatcccctctgccgaggggaggaagaaggcattctccacctgttctgcccacctcaccgtggtacttgtcttctatgggaccatcctgttcatgtacgggaagcccaagtccaaggacccattgggggcagacaaacaggatgtttcagacaagctcatctccttattttatggggtgctgacccccatgctgaaccccatcatctacagcctgaggaacaaggatgtgaaggccgctgtgaggaacctggtaggtcagaaatgcctgattcagtga

SEQ ID NO: 61 - DNA, 957 bp, Mus musculus
atggatgtatccaaccagacgactgtaacagaatttgtcctgttgggcctctccgcccaccccaaactggagaaaaccttcttcgtgctcatcttgtcaatgtacctggtgatcctgctgggcaacggggtcctcatcctggtgagcatcctcgactcccacctgcacacgcccatgtacttcttcctggggaacctctccttcctggacatctgctacaccacctcctccgttcccttagtccttgatggttttctgacccccaggaagaccatctccttctcgggctgtgccgtgcagatgtttctctccttcgccatgggagccacagagtgtgtgctcctgggcatgatggcgtttgatcgttatgtggccatctgcaacccccttagataccctgtggtcatgaacaaggctgcctatgtgcccatggccgtcagctcctgggtggctggcggtgctaactccttggtgcagatctcccttgcggtgcaattgcctttctgtggggacaatgtcatcaatcatttcatctgtgagatcctggcagtcttaaagctagcctgtgctgacatctccatcaatgtgatcagcatgggggtggccaatgtgattttcctgggggttccagttctgttcatctttgtctcctacatcttcatactctccaccatcttgaggatcccctctgctgaggggaggaagaaggcattctccacctgctctgcccacctcaccgtggtgatcattttttatgggaccatcctcttcatgtacgggaagcccaagtccaaggacccactgggggcagacaagcaggaccttgcagacaagctcatctccctcttctatggactgctgacccccatgctgaaccccatcatctacagcctgaggaacaaggacgtgaaggccgctgtgaggaacctggcaagtcacagatgcctcaccttctaa

SEQ ID NO: 62 - DNA, 960 bp, Mus musculus
atggaaagatccaacaagaccacccctgtgtccagcttcatcctcctgggtctctccgcccacccaaagctggagaaaaccttcttcgtgctcatcctgctgatgtacctggtgatcctgctgggcaatgtcgtcctcatcctggtgagcatcctcgactcccacctgcacacgcccatgtacttcttcctggggaacctctccttcctggacatctgctacaccacctcctccgtccccctcattctggacagcttcctgacccccaggaagaccatctccttctcgggctgtgccgtgcagatgtttctctccttcgccatgggagccacagagtgtgtgctcctgggcatgatggcgtttgatcgttatgtggccatctgcaacccccttagatatcctgtggtcatgagcaaggctgcctatgtgcccatggccgccggctcctgggtatctggaagcatcactgccacagtgcagatatctttagcaatgacactgcccttctgtggggacaatgtcatcaatcactttacatgtgagatcctggcagtcctgaaactggcctgtgctgacatctccatcaatgtcatcagcatggcagtggctaatgcaatgtttttgggggtccctgtcctcttcatctttgtctcctacatcttcatactctccaccatcctgaggatcccctctgccgaggggaggaagaaggcattctccacctgctctgcccacctcaccgtggtacttgtcttctatgggaccatcctgttcatgtacgggaagcccaaatccaaggacccattgggggcagacaaacaggaccttgcagacaagctcatctccctcttctatggagtggtgacccccatgctgaaccccatcatctacagcctgaggaacaaggatgtgaaggccgctgtgacaaacctggtgggccagaaacacttcaagtggtga

SEQ ID NO: 63 - DNA, 960 bp, Mus musculus
atggtacagggaaattggacctctgtcacagtgtttgtttttctgggattttcgcactaccccagaattgaagtcaccgtatttgtgctgtgtctgctgatgtatctgatcaccttgctgggaaatactattctgatctccatcactatccttgattctcatctgcacactcccatgtactttttcctcagcaacctctcatttctggacatctggtatacctcttctgctctcactcctatgctggcaaactttgtttcagggaaaaacaccatttccttctcgggatgtgcctcgcagatgtacttctctcttgccatgggctccactgaatgtgtgctcctctctatgatggcctatgacaggtatgtggccatctgtaacccactgagatatcccattattatgaacaggagggtctgtgtacagattgcaggcagctcctgggttacaggctgcctcactgccttggtggaaactggacctgtgattcatctgtctctctgtggtaacagcatcatcaatcatttcacctgtgaaattctggctctcttaaaattggcctgtggagacacttccatggtacagttaattatgctggtgatcagcattcttcttctcccattgccaatgttgctcatttgtgtctcctatgcatctatcctctccaacatcttgaggatcagctcaatggatggccgaagcaaagccttttcaacatgtgcagcccacttgactgtggtggttctgttctatgggacagctctctccatgtacctgaagccctcatctgtaaactcacaggaaattgataaatttatggcattaatatatactggactaacaccaatgctaaatcctattatctacagtctacggaacaaagaagtaaaaatggctgtgaaaaaattgttgatgaaaaaccccttcagtgctatcttaacttctgtcctcaaataa

SEQ ID NO: 64 - DNA, 930 bp, Homo sapiens
atgaaaatcaaccagacaatcctgaaggaattcattcttgttggcttttctgtgtacccacatgtacagacatttctttttgtggtcttcttttgtctctaccttctcacccttgcaggtaatctgatcatcatgggtctaacttgggtggacaggtccctccacacccctatgtatctcttccttagtgcactctccttctctgagacctgctatacgctgaccatcgtccccaagatgctggaagatctactggccaaggacagaagcatttcagtcacaggttgtagcttacagatgtgcttcttcttgggacttggtggcacaaactgtatcattctcactttgatgggatatgaccgcttcctggccatctgtaaccctctaagatatccactgcttatgaccaacattgtatgtggacaacttgtggcctctgcttgcactgcaggcttctttatctctcttacagagactgcactgatattcagggactctttctgcagacccaaccttgtcaaacacttcttctgccatatgctggcagttattaggctgtcttgtatagacagtaaccacacagaattcattataacactgatctcagtgtctggtttgctgggtacccttctgctcatcatcctgactgatgtcttcattatttctactgtcctcaggatcccttcagctgagggcaagcagaaggcattcaccacctgtgcctcccacctcaccgtggttataatccactttggttttgcatctattgtttatttgaagccagaagcctcaggagatgacacactcatagcagtcccttatactgtcattacccccttcctcagccccatcatattcagcctgaggaataaggacatgaaaaatgcttttagaagaatgatgggaaacacagttgccttgaaaaaataa

SEQ ID NO: 65 - DNA, 939 bp, Homo sapiens
atggaaccgctcaacagaacagaggtgtccgagttctttctgaaaggattttctggctacccagccctggagcatctgctcttccctctgtgctcagccatgtacctggtgaccctcctggggaacacagccatcatggcggtgagcgtgctagatatccacctgcacacgcccgtgtacttcttcctgggcaacctctctaccctggacatctgctacacgcccacctttgtgcctctgatgctggtccacctcctgtcatcccggaagaccatctcctttgctgtctgtgccatccagatgtgtctgagcctgtccacgggctccacggagtgcctgctactggccatcacggcctatgaccgctacctggccatctgccagccactcaggtaccgcgtgctcatgagccaccggctctgcgtgctgctgatgggagctgcctgggtcctctgcctcctcaagtcggtgactgagatggtcatctccatgaggctgcccttctgtggccaccacgtggtcagtcacttcacctgcaagatcctggcagtgctgaagctggcatgcggcaacacgtcggtcagcgaagacttcctgctggcgggctccatcctgctgctgcctgtacccctggcattcatctgcctgtcctacttgctcatcctggccaccatcctgagggtgccctcggccgccaggtgctgcaaagccttctccacctgcttggcacacctggctgtagtgctgcttttctacggcaccatcatcttcatgtacttgaagcccaagagtaaggaagcccacatctctgatgaggtcttcacagtcctctatgccatggtcacgaccatgctgaaccccaccatctacagcctgaggaacaaggaggtgaaggaggccgccaggaaggtgtggggcaggagtcgggcctccaggtga

SEQ ID NO: 66 - DNA, 960 bp, Homo sapiens
atggaaaaagccaatgagacctcccctgtgatggggttcgttctcctgaggctctctgcccacccagagctggaaaagacattcttcgtgctcatcctgctgatgtacctcgtgatcctgctgggcaatggggtcctcatcctggtgaccatccttgactcccgcctgcacacgcccatgtacttcttcctagggaacctctccttcctggacatctgcttcactacctcctcagtcccactggtcctggacagctttttgactccccaggaaaccatctccttctcagcctgtgctgtgcagatggcactctcctttgccatggcaggaacagagtgcttgctcctgagcatgatggcatttgatcgctatgtggccatctgcaacccccttaggtactccgtgatcatgagcaaggctgcctacatgcccatggctgccagctcctgggctattggtggtgctgcttccgtggtacacacatccttggcaattcagctgcccttctgtggagacaatgtcatcaaccacttcacctgtgagattctggctgttctaaagttggcctgtgctgacatttccatcaatgtgatcagcatggaggtgacgaatgtgatcttcctaggagtcccggttctgttcatctctttctcctatgtcttcatcatcaccaccatcctgaggatcccctcagctgaggggaggaaaaaggtcttctccacctgctctgcccacctcaccgtggtgatcgtcttctacgggaccttattcttcatgtatgggaagcctaagtctaaggactccatgggagcagacaaagaggatctttcagacaaactcatcccccttttctatggggtggtgaccccgatgctcaaccccatcatctatagcctgaggaacaaggatgtgaaggctgctgtgaggagactgctgagaccaaaaggcttcactcagtga

SEQ ID NO: 67 - DNA, 954 bp, Homo sapiens
atgggagagattaaccagacacttgtgtcagaatttcttcttctgggtctttctggatacccaaagattgagattgtttactttgctctcattctagttatgtacctagtgattctaattggcaatggtgttctaatcatagccagcatctttgattctcattttcacacaccaatgtacttcttcctgggcaacctctctttcctggatatctgctatacatcctcctctgttccctcaacattggtgagcttaatctcaaagaaaagaaacatttccttctctggatgtgcagtgcagatgttctttgggtttgcaatggggtcaacagaatgtctgcttcttggcatgatggcatttgatcgttatgtggccatctgcaacccactgagataccccatcatcctgagcaaggtggcgtatgtattgatggcttctgtgtcctggctgtccggtggaataaattcagctgtgcaaacattacttgccatgagactgcctttctgtgggaataatattatcaatcatttcgcatgtgaaatattagctgtcctcaagctggcctgtgctgatatatccctcaatattatcaccatggtgatatcaaatatggccttcctggttcttccactgatggtcatttttttctcctatatgttcatcctctacaccatcttgcaaatgaattcagccacaggaagacgcaaggcattttccacgtgctcagctcacctgactgtggtgatcatattttacggtaccatcttctttatgtatgcgaaaccgaagtctcaagacctgattggggaagaaaaattgcaagcattagacaagctcatttctctgttttatggggtagtgacacccatgctgaatcctatactctatagcttgagaaataaggatgtaaaagctgctgtaaaatatttgctgaacaaaaaaccaattcactaa

SEQ ID NO: 68 - DNA, 957 bp, Homo sapiens
atggacaagataaaccagacatttgtgagagaattcattcttctgggactctctggttaccccaaacttgagatcattttctttgctctgattctagttatgtacgtagtgattctaattggcaatggtgttctgatcatagcaagcatcttggattctcgtcttcacatgcccatgtacttcttcctgggcaacctctctttcctggatatctgctatacaacctcctccattccctcaacactggtgagcttaatctcaaagaaaagaaacatttccttctctggatgtgcagtgcagatgttctttgggtttgcaatggggtcaacagaatgtttcctccttggcatgatggcatttgatcgttatgtggccatctgtaaccctctgagataccccatcatcatgaacaaggtggtgtatgtactgctgacttctgtatcatggctttctggtggaatcaattcaactgtgcaaacatcacttgccatgcgatggcctttctgtgggaacaatattattaatcatttcttatgcgagatcttagctgtcctaaaattagcttgttctgatatatctgtcaatattgttaccctagcagtgtcaaatattgctttcctagttcttcctctgctcgtgatttttttctcctatatgttcatcctctacaccatcttgcgaacgaactcggccacaggaagacacaaggcattttctacatgctcagctcacctgactgtggtgatcatattttatggtaccatcttctttatgtatgcaaaacctaagtcccaggacctccttgggaaagacaacttgcaagctacagaggggcttgtttccatgttttatggggttgtgacccccatgttaaaccccataatctatagcttgagaaataaagatgtaaaagctgctataaaatatttgctgagcaggaaagctattaaccagtaa

SEQ ID NO: 69 - DNA, 963 bp, Homo sapiens
atggaaaggaccaacgattccacgtcgacagaatttttcctggtagggctttctgaccacccaaagctccagacagttttcttcgttctaattttgtggatgtacctgatgatcctgcttggaaatggagtccttatctcagttatcatctttgattctcacctgcacacccccatgtatttcttcctctgtaatctttccttcctcgacgtttgctacacaagttcctctgtcccactaattcttgccagctttctggcagtaaagaaaaaggtttccttctctgggtgtatggtgcaaatgtttatttcttttgccatgggggccacggagtgcatgatcttaggcacgatggcactggaccgctatgtggccatctgctacccactgagataccctgtcatcatgagcaagggtgcctatgtggccatggcagctgggtcctgggtcactgggcttgtggactcagtagtgcagacagcttttgcaatgcagttaccattctgtgctaataatgtcattaaacattttgtctgtgaaattctggctatcttgaaactggcctgtgctgatatttcaatcaatgtgattagtatgacagggtcgaatctgattgttctggttattccattgttagtaatttccatctcttacatatttattgttgccactattctgaggattccttccactgaaggaaaacataaggacttctccacctgctcagcccacctgacagtggtgattatattctatggaaccatcttcttcatgtacgcaaagcctgagtctaaagcctctgttgattcaggtaatgaagacatcattgaggccctcatctcccttttctatggagtgatgactcccatgcttaatcctctcatctatagtctgcgaaacaaggatgtaaaggctgctgtcaaaaacatactgtgtaggaaaaacttttctgatggaaaatga

SEQ ID NO: 70 - DNA, 957 bp, Homo sapiens
atggaatgggaaaaccacaccattctggtggaattttttctgaagggactttctggtcacccaagacttgagttactcttttttgtgctcatcttcataatgtatgtggtcatccttctggggaatggtactctcattttaatcagcatcttggaccctcaccttcacacccctatgtacttctttctggggaacctctccttcttggacatctgctacaccaccacctctattccctccacgctagtgagcttcctttcagaaagaaagaccatttccctttctggctgtgcagtgcagatgttcctcagcttggccatggggacaacagagtgtgtgcttctgggcgtgatggcctttgaccgctatgtggctatctgcaaccctctgagatatcccatcatcatgagtaaggatgcctatgtacccatggcagctgggtcctggatcataggagctgtcaattctgcagtacaaacagtgtttgtggtacaattgcctttctgcaggaataacatcatcaatcatttcacctgtgaaattctagctgtcatgaaactggcctgtgctgacatctcaggcaatgagttcatcctgcttgtgaccacaacattgttcctattgacacctttgttattaattattgtctcttacacgttaatcattttgagcatcttcaaaattagctcttcggaggggagaagcaaaccttcctctacctgctcagctcgtctgactgtggtgataacattctgtgggaccatcttcctcatgtacatgaagcccaagtctcaagagacacttaattcagatgacttggatgccactgacaaacttatattcatattctacagggtgatgactcccatgatgaatcctttaatctacagtcttagaaacaaggatgtgaaggaggcagtaaaacacctactgagaagaaaaaattttaacaagtaa

SEQ ID NO: 71 - DNA, 957 bp, Homo sapiens
atggaatgggaaaaccacaccattctggtggaattttttctgaagggactttctggtcacccaagacttgagttactcttttttgtgctcatcttcataatgtatgtggtcatccttctggggaatggtactctcattttaatcagcatcttggaccctcaccttcacacccctatgtacttctttctggggaacctctccttcttggacatctgctacaccaccacctctattccctccacgctagtgagcttcctttcagaaagaaagaccatttccctttctggctgtgcagtgcagatgttcctcggcttggccatggggacaacagagtgtgtgcttctgggcatgatggcctttgaccgctatgtggctatctgcaaccctctgagatatcccatcatcatgagtaaggatgcctatgtacccatggcagctgggtcctggatcataggagctgtcaattctgcagtacaatcagtgtttgtggtacaattgcctttctgcaggaataacatcatcaatcatttcacctgtgaaattctggctgtcatgaaactggcctgtgctgacatctcagacaatgagttcatcatgcttgtggccacaacattgttcatattgacacctttgttattaatcattgtctcttacacgttaatcattgtgagcatcttcaaaattagctcttccgaggggagaagcaaagcttcctctacctgttcagcccatctgactgtggtcataatattctatgggaccatcctcttcatgtacatgaagcccaagtctaaagagacacttaattcggatgacttggatgctaccgacaaaattatatccatgttctatggggtgatgactcccatgatgaatcctttaatctacagtcttagaaacaaggatgtgaaagaggcagtaaaacacctactgaacagaaggttctttagcaagtga

SEQ ID NO: 72 - DNA, 957 bp, Homo sapiens
atggaatgggaaaaccaaaccattctggtggaattttttctgaagggacattctgttcacccaaggcttgagttactcttttttgtgctaatcttcataatgtatgtggtcatccttctggggaatggtactctcattttaatcagcatcttggaccctcaccttcacacccctatgtacttctttctggggaacctctccttcttggacatctgctacaccaccacctctattccctccacactagtgagcttcctttcagaaagaaagaccatttccttttctggctgtgcagtgcagatgttccttggcttggccatggggacaacagagtgtgtgcttctgggcatgatggcctttgaccgctatgtggctatctgcaaccctctgagatatcccatcatcatgagcaagaatgcctatgtacccatggctgttgggtcctggtttgcagggattgtcaactctgcagtacaaactacatttgtagtacaattgcctttctgcaggaagaatgtcatcaatcatttctcatgtgaaattctagctgtcatgaagttggcctgtgctgacatctcaggcaatgagttcctcatgcttgtggccacaatattgttcacattgatgccactgctcttgatagttatctcttactcattaatcatttccagcatcctcaagattcactcctctgaggggagaagcaaagctttctctacctgctcagcccatctgactgtggtcataatattctatgggaccatcctcttcatgtatatgaagcccaagtctaaagagacacttaattcagatgacttggatgctaccgacaaaattatatccatgttctatggggtgatgactcccatgatgaatcctttaatctacagtcttagaaacaaggatgtgaaagaggcagtaaaacacctaccgaacagaaggttctttagcaagtga

SEQ ID NO: 73 - DNA, 945 bp, Homo sapiens
atggaaacaagaaattactctgccatgactgaattctttctggtggggctttcccaatatccagagctccagctttttctgttcctgctctgcctcatcatgtacatgataatcctcctgggaaatagcctcctcattatcatcaccatcttggattctcgcctccatactcccatgtatttctttcttggaaacctctcattcttggacatctgttacacatcctcatccattcctccaatgcttattatatttatgtctgagagaaaatccatctccttcattggctgtgctctgcagatggttgtgtcccttggcttgggctccactgagtgtgtcctcctggctgtgatggcctatgaccactatgtggccatctgcaacccactgaggtactccatcatcatgaacggagtgctgtatgtgcaaatggctgcatggtcctggatcataggctgtctgacctccctattgcacacagttctgacaatgatgttgcctttctgtgggaataatgtcattgatcatattacctgtgaaattttggcccttctaaaacttgtttgttcagatatcaccatcaatgtgcttatcatgacagtgacaaatattgtttcactggtgattcttctactgttaattttcatctcctatgtgtttattctctcttccatcctgagaattaattgtgctgagggaagaaagaaagccttctctacctgttcagcgcacttgattgtggtcatcttattctacggttcagccctttttatgtacatgaaacccaagtcaaagaacactaatacatctgatgagattattgggctgtcttatggagtggtaagcccaatgttaaatcccatcatctatagcctcaggaataaagaggtcaaagaggctgtaaagaaagtcctgagcagacatctgcatttattgaaaatgtga

SEQ ID NO: 74 - DNA, 951 bp, Homo sapiens
atgcaaggagaaaacttcaccatttggagcatttttttcttggagggattttcccagtacccagggttagaagtggttctcttcgtcttcagccttgtaatgtatctgacaacgctcttgggcaacagcactcttattttgatcactatcctagattcacgccttaaaacccccatgtacttattccttggaaatctctctttcatggatatttgttacacatctgcctctgttcctactttgctggtgaacttgctgtcatcccagaaaaccattatcttttctgggtgtgctgtacagatgtatctgtcccttgccatgggctccacagagtgtgtgctcctggccgtgatggcatatgaccgttatgtggccatttgtaacccgctgagatactccatcatcatgaacaggtgcgtctgtgcacggatggctacggtctcctgggtgacgggttgcctgaccgctctgctggaaaccagttttgccctgcagatacccctctgtgggaatctcatcgatcacttcacgtgtgaaattctggcggtgctaaagttagcttgcacaagttcactgctcatgaacaccatcatgctggtggtcagcattctcctcttgccaattccaatgctcttagtttgcatctcttacatcttcatcctttccactattctgagaatcacctcagcagagggaagaaacaaggctttttctacctgtggtgcccatttgactgtggtgattttgtattatggggctgccctctctatgtacctaaagccttcttcatcaaatgcacaaaaaatagacaaaatcatctcgttgctttacggagtgcttacccctatgttgaaccccataatttacagtttaagaaacaaggaagtcaaagatgctatgaagaaattgctgggcaaaataacattgcatcaaacacacgaacatctctga

SEQ ID NO: 75 - DNA, 944 bp, Homo sapiens
atgttaatgaattactctagtgccactgaattttatctccttggcttccctggctctgaagaactacatcatatcctttttgctatattcttctttttctacttggtgacattaatgggaaacacagtcatcatcatgattgtctgtgtggataaacgtctgcagtcccccatgtatttcttcctcggccacctctctgccctggagatcctggtcacaaccataatcgtccccgtgatgctttggggattgctgctccctgggatgcagacaatatatttgtctgcctgtgttgtccagctcttcttgtaccttgctgtggggacaacagagttcgcattacttggagcaatggctgtggaccgttatgtggctgtctgtaaccctctgaggtacaacatcattatgaacagacacacctgcaactttgtggttcttgtgtcatgggtgtttgggtttctttttcaaatctggccggtctatgtcatgtttcagcttacttactgcaaatcaaatgtggtgaacaattttttttgtgaccgagggcaattgctcaaactatcctgcaataatactcttttcacggagtttatcctcttcttaatggctgtttttgttctctttggttctttgatccctacaattgtctccaacgcctacatcatctccaccattctcaagatcccgtcatcctctggccggaggaaatccttctccacttgtgcctcccacttcacctgtgttgtgattggctacggcagctgcttgtttctctacgtgaaacccaagcaaacgcaggcagctgattacaattgggtagtttccctgatggtttcagtagtaactcctttcctcaatcctttcatcttcaccctccggaatgataaagtcatagaggcccttcgggatggggtgaaacgctgctgtcaactattcaggaatta

SEQ ID NO: 76 - DNA, 933 bp, Homo sapiens
atgatggacaaccactctagtgccactgaattccaccttctaggcttccctgggtcccaaggactacaccacattctttttgctatattctttttcttctatttagtgacattaatgggaaacacggtcatcattgtgattgtctgtgtggataaacgtctgcagtcccccatgtatttcttcctcagccacctctctaccctggagatcctggtcacaaccataattgtccccatgatgctttggggattgctcttcctgggatgcagacagtatctttctctacatgtatcgctcaacttttcctgtgggaccatggagtttgcattacttggagtgatggctgtggaccgttatgtggctgtgtgtaaccctttgaggtacaacatcattatgaacagcagtacctgtatttgggtggtaatagtgtcatgggtgtttggatttctttctgaaatctggcccatctatgccacatttcagtttaccttccgcaaatcaaattcattagaccatttttactgtgaccgagggcaattgctcaaactgtcctgcgataacactcttctcacagagtttatccttttcttaatggctgtttttattctcattggttctttgatccctacgattgtctcctacacctacattatctccaccatcctcaagatcccgtcagcctctggccggaggaaagccttctccacttttgcctcccacttcacctgtgttgtgattggctatggcagctgcttgtttctctacgtgaaacccaagcaaacacagggagttgagtacaataagatagtttccctgttggtttctgtgttaacccccttcctgaatcctttcatctttactcttcggaatgacaaagtcaaagaggccctccgagatgggatgaaacgctgctgtcaactcctgaaagattag

SEQ ID NO: 77 - DNA, 939 bp, Homo sapiens
atgagaaatggcacagtaatcacagaattcatcctgctaggctttcctgttatccaaggactacaaacacctctctttattgcaatctttctcacctacatattaacccttgcaggcaatgggcttattattgccactgtgtgggctgagcccaggctacaaattccaatgtacttcttcctttgtaacttgtctttcttagaaatctggtacaccaccacagtcatccccaaactgctaggaacctttgtagtggcaagaacagtaatctgcatgtcctgctgcctgctgcaggcattcttccacttcttcgtgggcaccaccgagttcttgatcctcactatcatgtcttttgaccgctacctcaccatctgcaatccccttcaccaccccaccatcatgaccagcaaactctgcctgcagctggccctgagctcctgggtggtgggcttcaccattgtcttttgtcagacgatgctgctcatccagttgccattctgtggcaataatgttatcagtcatttctactgtgatgttgggcccagtttgaaagccgcctgcatagacaccagcattttggaactcctgggcgtcatagcaaccatccttgtgatcccagggtcacttctctttaatatgatttcttatatctacattctgtccgcaatcctacgaattccttcagccactggccaccaaaagactttctctacctgtgcctcgcacctgacagttgtctccctgctctacggggctgttctgttcatgtacctaagacccacagcacactcctcctttaagattaataaggtggtgtctgtgctaaatactatcctcaccccccttctgaatccctttatttatactattagaaacaaggaggtgaagggagccttaagaaaggcaatgacttgcccaaagactggtcatgcaaagtaa

SEQ ID NO: 78 - DNA, 942 bp, Homo sapiens
atgggagacaggggaacaagcaatcactcagaaatgactgacttcattcttgcaggcttcagggtacgcccagagctccacattctcctcttcctgctatttttgtttgtttatgccatgatccttctagggaatgttgggatgatgaccattattatgactgatcctcggctgaacacaccaatgtattttttcctaggcaatctctccttcattgatcttttctattcatctgttattgaacccaaggctatgatcaacttctggtctgaaaacaagtctatctcctttgcaggctgtgtggcccagctctttctctttgccctcctcattgtgactgagggatttctcctggcggccatggcttatgaccgctttattgccatctgcaaccctctgctctactctgttcaaatgtccacacgtctgtgtactcagttggtggctggttcctatttttgtggctgcattagctcagttattcagactagcatgacatttactttatctttttgcgcttctcgggctgttgaccacttttactgtgattctcgcccacttcagagactgtcttgttctgatctctttatccatagaatgatatctttttccttatcatgtattattatcttgcctactatcatagtcattatagtatcttacatgtatattgtgtccacagttctaaagatacattctactgagggacataagaaagccttctccacctgcagctctcacctgggagttgtgagtgtgctgtatggtgctgtcttttttatgtatctcactcctgacagatttcctgagctgagtaaagtggcatccttatgttactccctagtcactcccatgttgaatcctttgatttactctctgaggaacaaagatgtccaagaggctctaaaaaaatttctagagaagaaaaatattattctttga

SEQ ID NO: 79 - DNA, 960 bp, Homo sapiens
atgttcccggcaaattggacatctgtaaaagtatttttcttcctgggattttctcactaccccaaagttcaggtcatcatatttgcggtgtgcttgctgatgtacctgatcaccttgctgggcaacatttttctgatctccatcaccattctagattcccacctgcacacccctatgtacctcttcctcagcaatctctcctttctggacatctggtactcctcttctgccctctctccaatgctggcaaactttgtttcagggagaaacactatttcattctcagggtgcgccactcaggtgtacctctcccttgccatgggctccactgagtgtgtgctcctgcccatgatggcatatgaccggtatgtggccatctgcaaccccctgagataccctatcatcatgaataggagaacctgtgtgcagattgcagctggctcctggatgacaggctgtctcactgccatggtggaaatgatgtctgtgctgccactgtctctctgtggtaatagcatcatcaatcatttcacttgtgaaattctggccatcttgaaattggtttgtgtggacacctccctggtgcagttaatcatgctggtgatcagtgtacttcttctccccatgccaatgctactcatttgtatctcttatgcatttatcctcgccagtatcctgagaatcagctcagtggaaggtcgaagtaaagccttttcaacgtgcacagcccacctgatggtggtagttttgttctatgggatggctctctccatgcacctgaagccctccgctgtagattcacaggaaatagacaaatttatggctttggtgtatgccggacaaacccccatgttgaatcctatcatctatagtctacggaacaaagaggtgaaagtggccttgaaaaaattgctgattagaaatcattttaatactgccttcatttccatcctcaaataa

SEQ ID NO: 80 - DNA, 960 bp, Rattus norvegicus
atggtacagggaaattggtcctctgtcacagtgtttttcttcctgggattttctcagtaccccagaattgaagtcaccatatttgtgctgtgtctgctgatgtacctgatcaccttgctgggaaatactattctgatctccatcactatccttgattctcacctgcacactcccatgtactttttccttagcaacctctcctttctggacatctggtatacctcttctgctctcactcctatgctggcaaactttgtttcaggggaaaacaccatctcattctcaggatgtgcttttcagatgtacttctctcttgccatgggctccacggaatgtgtgctcctctctatgatggcctatgacaggtacgtggccatctgcaaccccctgagatatcccatcattatgaacaggagagtctgtgtacagattgcaggcagctcctgggttacaggctgcctcactgccttggtggaaactggacctgtgattcatctgtctctctgtggtaataacatcatcaatcatttcacctgtgaaattctggctctcttaaaaatggcttgtggagacactgccatggtgcagttaattatgttagtgatcagcatccttcttctcccattgccaatgttgctcatttgtgtctcctatgcatccatcctctccaacatcttgaggatcagctcagtggatggccgaagcaaagccttttcaacatgtgcagcccacttgactgtggtggttctgttctatgagacagctctctccatgtacctgaagcccttatctgtaaactcacaggaaatagataaatttatggcattgatatatgttggactaacaccaatgctaaatcctattatctacagtctacggaacaaagaagtcaaaatggctgtgaaaaaattgttgattaaaaatcccttcactgctatcttaacttctgtcctcaaataa

SEQ ID NO: 81 - DNA, 13 bp, Artificial Sequence
  ENHANCER SEQUENCE
aactttttaatga

SEQ ID NO: 82 - DNA, 21 bp, Artificial Sequence
  ENHANCER SEQUENCE
acataactttttaatgagtct

SEQ ID NO: 83 - DNA, 11 bp, Artificial Sequence
  ENHANCER SEQUENCE, WHERE Y=C/T AND R=A/G
yttttaatgar